(19) United States
(12) Patent Application Publication
(10) Pub. No.: US 2012/0010186 A1
(43) Pub. Date: Jan. 12, 2012

(54) HETEROCYCLIC COMPOUNDS AS INHIBITORS OF STEAROYL-COENZYME A DELTA-9 DESATURASE

(75) Inventors: Nicolas Lachance, Pierrefonds (CA); Serge Leger, Notre-Dame-De-I'Ile-Perrot (CA); Renata M. Oballa, Kirkland (CA); David Powell, Verdun (CA); Geoffrey K. Tranmer, Westmount (CA); Evelyn Martins, Vaudreuil-Dorion (CA); Yves Gareau, Notre-Dame-De-I'Ile-Perrot (CA)

(73) Assignee: MERCK FROSST CANADA LTD., Kirkland, QC (CA)

(21) Appl. No.: 13/255,922

(22) PCT Filed: Mar. 18, 2010
(86) PCT No.: PCT/CA2010/000430

§ 371 Date: Sep. 12, 2011

Related U.S. Application Data
(60) Provisional application No. 61/210,776, filed on Mar. 23, 2009.

(51) Int. Cl. A61K 31/427; C07D 487/10; C07D 471/10; A61P 3/06; A61K 31/422; A61K 31/438; A61K 31/506; A61P 3/10; C07D 487/04; A61K 31/433

(52) U.S. Cl. 514/210.2; 548/181; 548/138; 548/246; 548/147; 546/16; 544/331; 514/370; 514/363; 514/380; 514/275

(57) ABSTRACT

Heterocyclic compounds of structural formula I are inhibitors of stearoyl-coenzyme A delta-9 desaturase (SCD). The compounds of the present invention are useful for the prevention and treatment of conditions related to abnormal lipid synthesis and metabolism, including cardiovascular disease; atherosclerosis; obesity; diabetes; neurological disease; Metabolic Syndrome; insulin resistance; cancer, liver steatosis; and non-alcoholic steatohepatitis.

### FIELD OF THE INVENTION

The present invention relates to heterocyclic compounds which are inhibitors of stearoyl-coenzyme A delta-9 desaturase (SCD) and the use of such compounds to control, prevent and/or treat conditions or diseases mediated by SCD activity. The compounds of the present invention are useful for the control, prevention and treatment of conditions and diseases related to abnormal lipid synthesis and metabolism, including cardiovascular disease; atherosclerosis; obesity; diabetes; neurological disease; Metabolic Syndrome; insulin resistance; cancer; liver steatosis; and non-alcoholic steatohepatitis.

### BACKGROUND OF THE INVENTION

At least three classes of fatty acyl-coenzyme A (CoA) desaturases (delta-5, delta-6 and delta-9 desaturases) are responsible for the formation of double bonds in mono- and polyunsaturated fatty acyl-CoAs derived from either dietary sources or de novo synthesis in mammals. The delta-9 specific stearoyl-CoA desaturases (SCDs) catalyze the rate-limiting formation of the cis-double bond at the C₉-C₁₀ position in monounsaturated fatty acyl-CoAs. The preferred substrates are stearoyl-CoA and palmitoyl-CoA, with the resulting oleoyl and palmitoleoyl-CoA as the main components in the biosynthesis of phospholipids, triglycerides, cholesterol esters and wax esters (Dobrzyn and Natami, Obesity Reviews, 6: 169-174 (2005)).

The rat liver microsomal SCD protein was first isolated and characterized in 1974 (Strittmatter et al., PNAS, 71: 4565-4569 (1974)). A number of mammalian SCD genes have since been cloned and studied from various species. For example, two genes have been identified from rat (SCD1 and SCD2, Thiede et al., J. Biol. Chem., 261, 13230-13235 (1986)), Mihara, K., J. Biochem. (Tokyo), 108: 1022-1029 (1990)); four genes from mouse (SCD1, SCD2, SCD3 and SCD4) (Miyazaki et al., J. Biol. Chem., 278: 33904-33911 (2003)); and two genes from human (SCD1 and ACOD4 (SCD2)), (Zhang, et al., Biochem. J., 340: 255-264 (1991); Beiraghi, et al., Gene, 309: 11-21 (2003); Zhang et al., Biochem. J., 388: 135-142 (2005)). The involvement of SCDs in fatty acid metabolism has been known in rats and mice since the 1970's (Oshino, N., Arch. Biochem. Biophys., 149: 378-387 (1972)). This has been further supported by the biological studies of a) Asebia mice that carry the natural mutation in the SCD1 gene (Zheng et al., Nature Genetics, 23: 268-270 (1999)), b) SCD1-null mice from targeted gene deletion (Ntambi, et al., PNAS, 99: 11482-11486 (2002), and c) the suppression of SCD1 expression during leptin-induced weight loss (Cohen et al., Science, 297: 240-243 (2002)). The potential benefits of pharmacological inhibition of SCD activity has been demonstrated with anti-sense oligonucleotide inhibitors (ASO) in mice (Jiang, et al., J. Clin. Invest., 115: 1030-1038 (2005)). ASO inhibition of SCD activity reduced fatty acid synthesis and increased fatty acid oxidation in primary mouse hepatocytes. Treatment of mice with SCD-ASOs resulted in the prevention of diet-induced obesity, reduced body adiposity, hepatomegaly, steatosis, postprandial plasma insulin and glucose levels, reduced de novo fatty acid synthesis, decreased the expression of lipogenic genes, and increased the expression of genes promoting energy expenditure in liver and adipose tissues. Thus, SCD inhibition represents a novel therapeutic strategy in the treatment of obesity and related metabolic disorders.

There is compelling evidence to support that elevated SCD activity in humans is directly implicated in several common disease processes. For example, there is an elevated hepatic lipogenesis to triglyceride secretion in non-alcoholic fatty liver disease patients (Diraison, et al., Diabetes Metabolism, 29: 478-485 (2003)); Donnelly, et al., J. Clin. Invest., 115: 1343-1351 (2005)). Elevated SCD activity in adipose tissue is closely coupled to the development of insulin resistance (Sjogren, et al., Diabetologia, 51(2): 328-35 (2007)). The postprandial de novo lipogenesis is significantly elevated in obese subjects (Marques-Lopes, et al., American Journal of Clinical Nutrition, 73: 252-261 (2001)). Knockout of the SCD gene ameliorates Metabolic Syndrome by reducing plasma triglycerides, reducing weight gain, increasing insulin sensitivity, and reduces hepatic lipid accumulation (MacDonald, et al., Journal of Lipid Research, 49(1): 217-29 (2007)). There is a significant correlation between a high SCD activity and an increased cardiovascular risk profile including elevated plasma triglycerides, a high body mass index and reduced plasma HDL (Attie, et al., J. Lipid Res., 43: 1899-1907 (2002)). SCD activity plays a key role in controlling the proliferation and survival of human transformed cells (Scaglia and Igal, J. Biol. Chem., (2005)). RNA interference of SCD-1 reduces human tumor cell survival (Morgan-Lappe, et al., Cancer Research, 67(9): 4390-4398 (2007)).

Other than the above mentioned anti-sense oligonucleotides, inhibitors of SCD activity include non-selective thia-fatty acid substrate analogs [B. Behrouzian and P. H. Buist, Prostaglandins, Leukotrienes, and Essential Fatty Acids, 68: 107-112 (2003)], cyclopropenoid fatty acids (Raju and Reiser, J. Biol. Chem., 242: 379-384 (1967)), certain conjugated long-chain fatty acid isomers (Park, et al., Biochim. Biophys. Acta, 1486: 285-292 (2000)), and a series of heterocyclic derivatives disclosed in published international patent application publications WO 2005/011653, WO 2005/011654, WO 2005/011656, WO 2005/011656, WO 2005/011657, WO 2006/014168, WO 2006/034279, WO 2006/034312, WO 2006/034315, WO 2006/034338, WO 2006/034341, WO 2006/034440, WO 2006/034441, WO 2006/034446, WO 2006/086445; WO 2006/086447; WO 2006/101521; WO 2006/125178; WO 2006/125179; WO 2006/125180; WO 2006/125181; WO 2006/125194; WO 2007/044085; WO 2007/046867; WO 2007/046868; WO 2007/050124; WO 2007/130075; WO 2007/136746; WO 2008/036715; WO 2008/074835; WO 2008/127349; and U.S. Pat. Nos. 7,456,180 and 7,390,813; all assigned to Xenon Pharmaceuticals, Inc. or Xenon Pharmaceuticals, Inc./Novartis AG.

A number of international patent applications assigned to Merck Frosst Canada Ltd. that disclose SCD inhibitors useful for the treatment of obesity and Type 2 diabetes have also published: WO 2006/130986 (14 Dec. 2006); WO 2007/009236 (25 Jan. 2007); WO 2007/056846 (24 May 2007); WO 2007/071023 (28 Jun. 2007); WO 2007/134457 (29 Nov. 2007); WO 2007/143823 (21 Dec. 2007); WO 2007/143824 (21 Dec. 2007); WO 2008/017161 (14 Feb. 2008); WO 2008/046226 (24 Apr. 2008); WO 2008/064474 (5 Jun. 2008); WO 2008/089580 (31 Jul. 2008); WO 2008/128335 (30 Oct. 2008); WO 2008/141455 (27 Nov. 2008); US 2008/0132542 (5 Jun. 2008); and US 2008/0182838 (31 Jul. 2008).

WO 2008/003753 (assigned to Novartis) discloses a series of pyrazolo[1,5-a]pyrimidine analogs as SCD inhibitors; WO 2007/143597 and WO 2008/024390 (assigned to Novartis AG and Xenon Pharmaceuticals) disclose heterocyclic derivatives as SCD inhibitors; and WO 2008/096746 (assigned to Takeda Pharmaceutical) disclose Spiro compounds as SCD inhibitors.

Additional international patent applications disclosing SCD inhibitors have published: WO 2008/062276 (Glenmark; 29 May 2008); WO 2008/029266 (Glenmark; 13 Mar. 2008); WO 2008/003753 (Biovitrum AB; 10 Jan. 2008); WO 2008/135141 (Sanofi-Aventis; 13 Nov. 2008); WO 2008/157844 (Sanofi-Aventis; 24 Dec. 2008); WO 2008/104524 (SKB; 4 Sep. 2008); WO 2008/074834 (SKB; 26 Jun. 2008); WO 2008/074833 (SKB; 26 Jun. 2008); WO 2008/074832 (SKB; 26 Jun. 2008); and WO 2008/074824 (SKB; 26 Jun. 2008).

Small molecule SCD inhibitors have also been described by (a) G. Liu, et al., “Discovery of Potent, Selective, Orally Bioavailable SCD1 Inhibitors,” in J. Med. Chem., 50: 3086-3100 (2007); (b) H. Zhao, et al., “Discovery of 1-(4-phenoxypiperidin-1-yl)-2-arylaminoethanone SCD 1 inhibitors,” Bioorg. Med. Chem. Lett., 17: 3388-3391 (2007); and (c) Z. Xin, et al., “Discovery of piperidine-aryl urea-based stearoyl-CoA desaturase 1 inhibitors,” Bioorg. Med. Chem. Lett., 18: 4298-4302 (2008).

The present invention is concerned with novel heterocyclic compounds as inhibitors of stearoyl-CoA delta-9 desaturase which are useful in the treatment and/or prevention of various conditions and diseases mediated by SCD activity including those related, but not limited, to elevated lipid levels, as exemplified in non-alcoholic fatty liver disease, cardiovascular disease, obesity, diabetes, metabolic syndrome, and insulin resistance.

The role of stearoyl-coenzyme A desaturase in lipid metabolism has been described by M. Miyazaki and J. M. Ntambi, Prostaglandins, Leukotrienes, and Essential Fatty Acids, 68: 113-121 (2003). The therapeutic potential of the pharmacological manipulation of SCD activity has been described by A. Dobrzyn and J. M. Ntambi, in “Stearoyl-CoA desaturase as a new drug target for obesity treatment,” Obesity Reviews, 6: 169-174 (2005).

### SUMMARY OF THE INVENTION

The present invention relates to heterocyclic compounds of structural formula I:

W-Het-Ar (I)

These heterocyclic compounds are effective as inhibitors of SCD. They are therefore useful for the treatment, control or prevention of disorders responsive to the inhibition of SCD, such as diabetes, insulin resistance, lipid disorders, obesity, atherosclerosis, and metabolic syndrome.

The present invention also relates to pharmaceutical compositions comprising the compounds of the present invention and a pharmaceutically acceptable carrier.

The present invention also relates to methods for the treatment, control, or prevention of disorders, diseases, or conditions responsive to inhibition of SCD in a subject in need thereof by administering the compounds and pharmaceutical compositions of the present invention.

The present invention also relates to methods for the treatment, control, or prevention of Type 2 diabetes, insulin resistance, obesity, lipid disorders, atherosclerosis, and metabolic syndrome by administering the compounds and pharmaceutical compositions of the present invention.

The present invention also relates to methods for the treatment, control, or prevention of obesity by administering the compounds of the present invention in combination with a therapeutically effective amount of another agent known to be useful to treat the condition.

The present invention also relates to methods for the treatment, control, or prevention of Type 2 diabetes by administering the compounds of the present invention in combination with a therapeutically effective amount of another agent known to be useful to treat the condition.

The present invention also relates to methods for the treatment, control, or prevention of atherosclerosis by administering the compounds of the present invention in combination with a therapeutically effective amount of another agent known to be useful to treat the condition.

The present invention also relates to methods for the treatment, control, or prevention of lipid disorders by administering the compounds of the present invention in combination with a therapeutically effective amount of another agent known to be useful to treat the condition.

The present invention also relates to methods for treating metabolic syndrome by administering the compounds of the present invention in combination with a therapeutically effective amount of another agent known to be useful to treat the condition.

### DETAILED DESCRIPTION OF THE INVENTION

The present invention is concerned with heterocyclic compounds useful as inhibitors of SCD. Compounds of the present invention are described by structural formula I:

W-Het-Ar (I)

and pharmaceutically acceptable salts thereof; wherein
Het is a heterobicyclic ring system selected from the group consisting of:

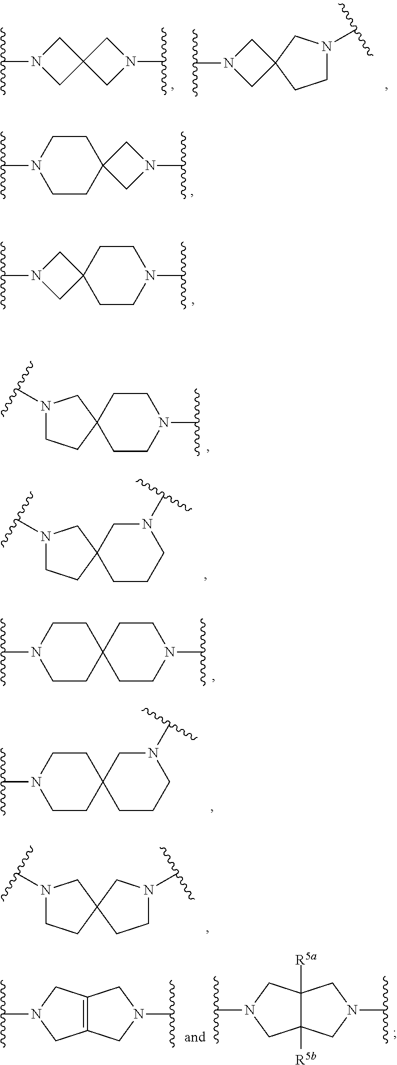

W is heteroaryl selected from the group consisting of:

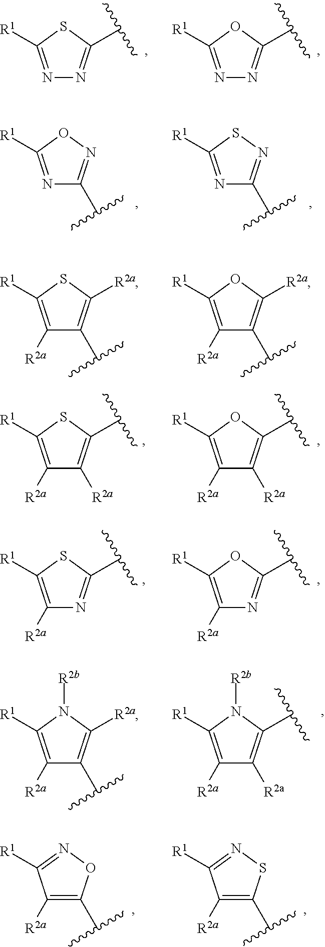

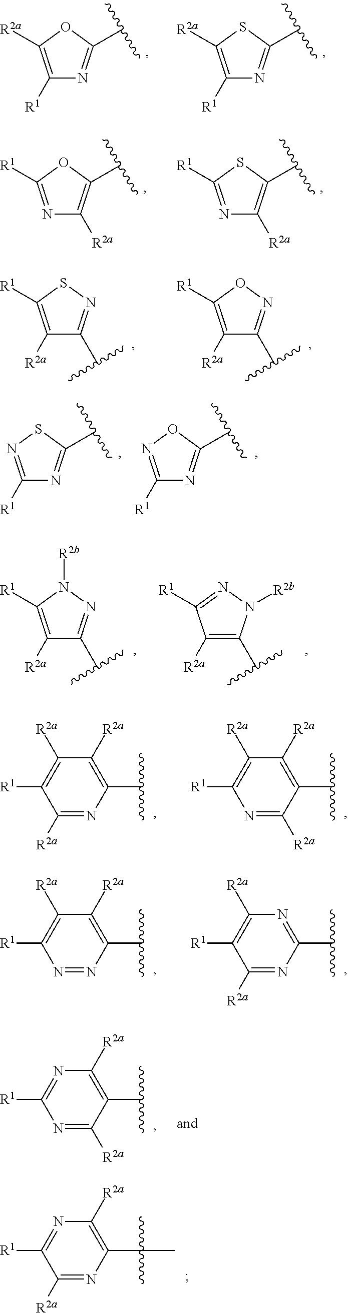

R¹ is heteroaryl selected from the group consisting of:

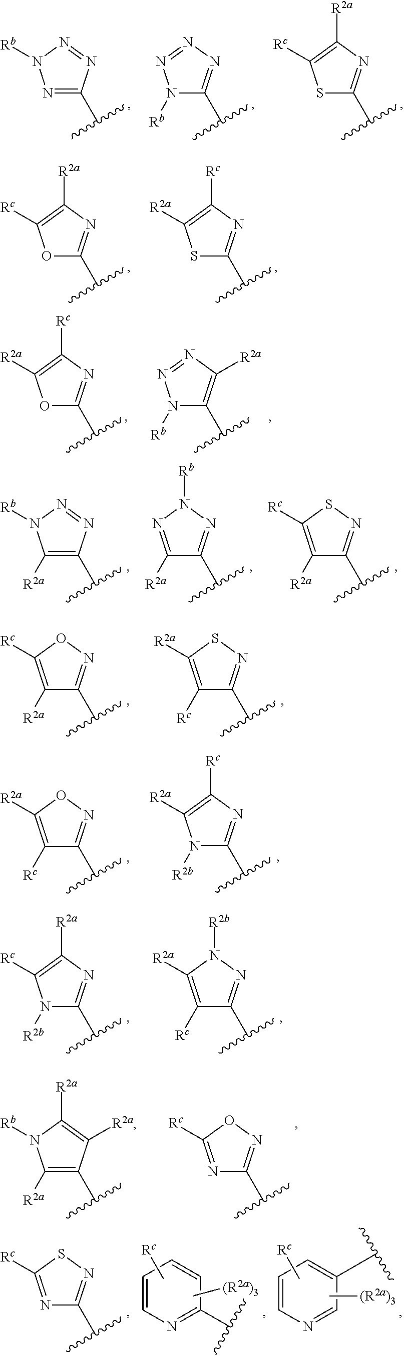

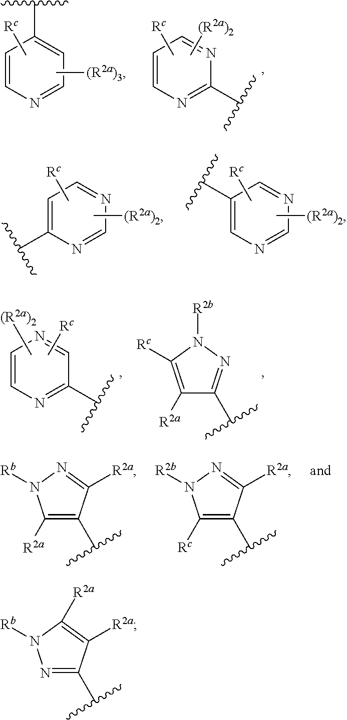

wherein
R^{b} is —(CH₂)ᵣCO₂H, —(CH₂)ᵣCO₂C₁₋₃ alkyl, —(CH₂)ᵣ—Z—(CH₂)ₚCO₂H, or —(CH₂)ᵣ—Z—(CH₂)ₚCO₂C₁₋₃ alkyl;
R^{c} is —(CH₂)ₘCO₂H, —(CH₂)ₘCO₂C₁₋₃ alkyl, —(CH₂)ₘ—Z—(CH₂)ₚCO₂H, or —(CH₂)ₘ—Z—(CH₂)ₚCO₂C₁₋₃ alkyl;

### Z is O, S(O)_{q}, or NR⁴;

each R^{2a} is independently selected from the group consisting of:
hydrogen,
halogen,
hydroxy,
cyano,
C₁₋₄ alkyl, optionally substituted with one to five fluorines,
C₁₋₄ alkoxy, optionally substituted with one to five fluorines,
C₁₋₄ alkylthio, optionally substituted with one to five fluorines,
C₁₋₄ alkylsulfonyl, optionally substituted with one to five fluorines, carboxy,
C₁₋₄ alkyloxycarbonyl, and
C₁₋₄ alkylcarbonyl;
   each R^{2b} is independently selected from the group consisting of:
hydrogen,
C₁₋₄ alkyl, optionally substituted with one to five fluorines,
C₁₋₄ alkylsulfonyl, optionally substituted with one to five fluorines,
C₁₋₄ alkyloxycarbonyl, and
C₁₋₄ alkylcarbonyl;
   Ar is phenyl, naphthyl, thienyl, or pyridyl optionally substituted with one to five R³ substituents;
   each R³ is independently selected from the group consisting of:
halogen,
cyano,
C₁₋₆ alkyl, optionally substituted with one to five fluorines,
C₁₋₆ alkoxy, optionally substituted with one to five fluorines, —OCH₂C₃₋₆ cycloalkyl,
C₁₋₆ alkylthio, optionally substituted with one to five fluorines,
C₁₋₆ alkylsulfonyl, optionally substituted with one to five fluorines, and
phenyl, optionally substituted with one to three substituents independently selected from halogen, C₁₋₄ alkyl, cyano, trifluoromethyl, and trifluoromethoxy;
   each R⁴ is independently selected from the group consisting of
hydrogen,
C₁₋₆ alkyl,
(CH₂)ₙ-phenyl,
(C₁₋₁₂)ₙ-heteroaryl,
(CH₂)ₙ-naphthyl, and
(CH₂)ₙC₃₋₇ cycloalkyl;
   wherein alkyl, phenyl, heteroaryl, naphthyl, and cycloalkyl are optionally substituted with one to three groups independently selected from halogen, C₁₋₄ alkyl, and C₁₋₄ alkoxy;
   R^{5a} and R^{5b} are each independently selected from the group consisting of:
hydrogen,
fluorine,
hydroxy,
C₁₋₃ alkyl, optionally substituted with one to five fluorines, and
C₁₋₄ alkylcarbonyloxy;
   m is an integer from 0 to 3;
   n is an integer from 0 to 2;
   p is an integer from 1 to 3;
   q is an integer from 0 to 2; and
   r is an integer from 1 to 3.

In one embodiment of the compounds of the present invention, Het is

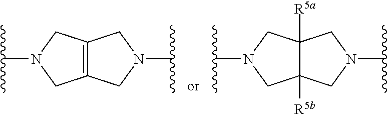

wherein R^{5a} and R^{5b} are as defined above. In a class of this first embodiment, Het is

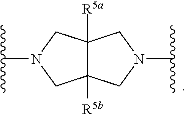

In a subclass of this class, R^{5a} and R^{5b} are each hydrogen. In another subclass of this class, R^{5a} is hydrogen, and R^{5b} is fluorine, hydroxy, or methyl.

In a second class of this first embodiment, Het is

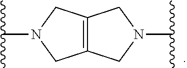

In a second embodiment of the compounds of the present invention, Het is

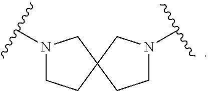

In a third embodiment of the compounds of the present invention, Het is selected from the group consisting of:

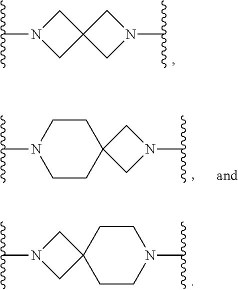

In a class of this third embodiment of the compounds of the present invention, Het is

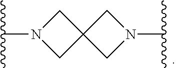

In a fourth embodiment of the compounds of the present invention, Ar is phenyl optionally substituted with one to three substituents each independently selected from R³ as defined above. In a class of this fourth embodiment, each R³ is independently methyl, halogen, trifluoromethyl, or trifluoromethoxy.

In a fifth embodiment of the compounds of the present invention, W is heteroaryl selected from the group consisting of:

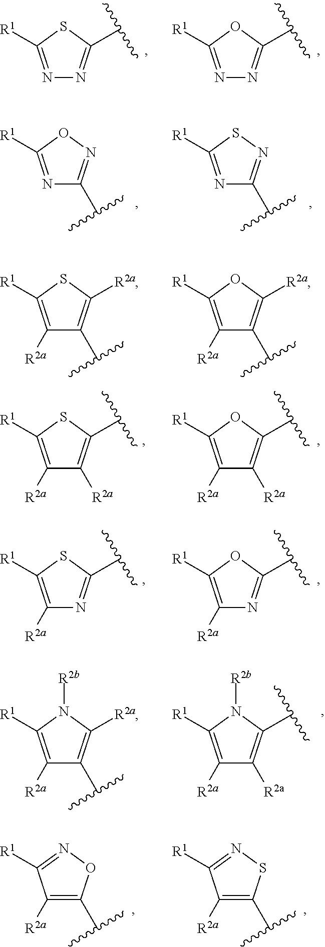

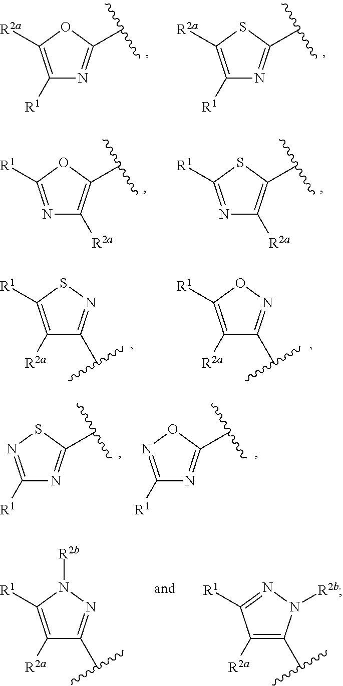

wherein R¹ and R^{2a} are as defined above. In a class of this fifth embodiment, R^{2a} and R^{2b} are each hydrogen.

In another class of this fifth embodiment, W is heteroaryl selected from the group consisting of:

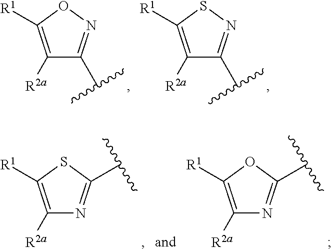

wherein R¹ and R^{2a} are as defined above. In a subclass of this class, R^{2a} is hydrogen.

In a sixth embodiment of the compounds of the present invention, W is heteroaryl selected from the group consisting of:

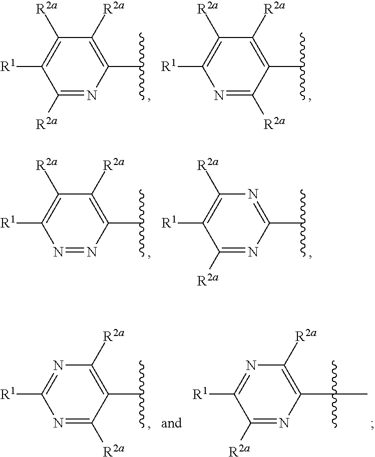

wherein R¹ and R^{2a} are as defined above. In a class of this sixth embodiment, each R^{2a} is hydrogen. In another class of this sixth embodiment, W is

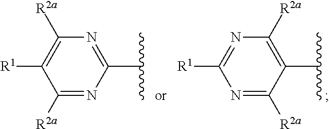

wherein R¹ and R^{2a} are as defined above. In a subclass of this class, each R^{2a} is hydrogen.

In a seventh embodiment of the compounds of the present invention, R¹ is heteroaryl selected from the group consisting of:

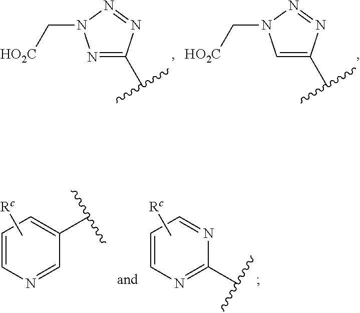

wherein R^{c} is —CO₂H, —CO₂C₁₋₃ alkyl, —CH₂CO₂H, or —CH₂CO₂C₁₋₃ alkyl. In a class of this seventh embodiment, R¹ is

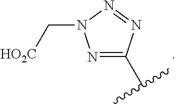

In an eighth embodiment of the compounds of the present invention, W is heteroaryl selected from the group consisting of:

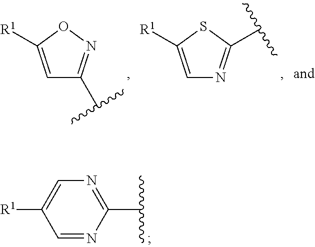

and R¹ is heteroaryl selected from the group consisting of:

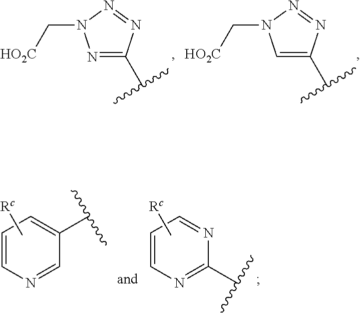

wherein R^{c} is —CO₂H, —CO₂C₁₋₃ alkyl, —CH₂CO₂H, or —CH₂CO₂C₁₋₃ alkyl.

In a class of this eighth embodiment, W is

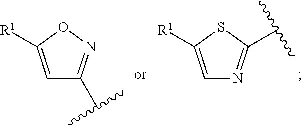

### and R¹ is

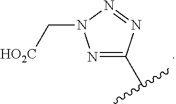

In a ninth embodiment of the compounds of the present invention, Het is

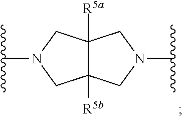

Ar is phenyl optionally substituted with one to three substituents each independently selected from methyl, halogen, trifluoromethyl, and trifluoromethoxy;
W is heteroaryl selected from the group consisting of:

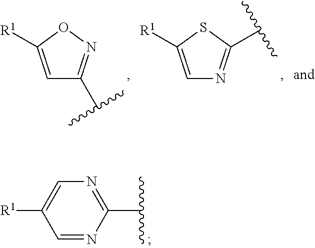

and R¹ is heteroaryl selected from the group consisting of:

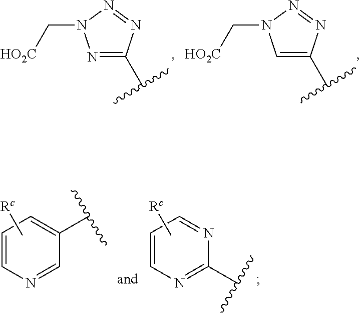

wherein R^{c} is —CO₂H, —CO₂C₁₋₃ alkyl, —CH₂CO₂H, or —CH₂CO₂C₁₋₃ alkyl.

In a class of this ninth embodiment, W is

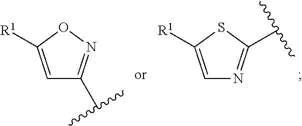

### and R¹ is

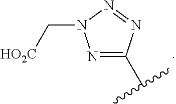

and R^{5a} and R^{5b} are each hydrogen.

In a tenth embodiment of the compounds of the present invention, Het is

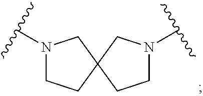

Ar is phenyl optionally substituted with one to three substituents each independently selected from methyl, halogen, trifluoromethyl, and trifluoromethoxy;
W is heteroaryl selected from the group consisting of:

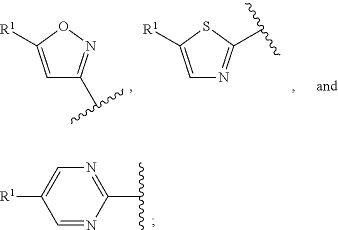

and R¹ is heteroaryl selected from the group consisting of:

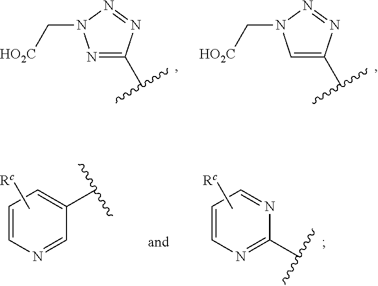

wherein R^{c} is —CO₂H, —CO₂C₁₋₃ alkyl, —CH₂CO₂H, or —CH₂CO₂C₁₋₃ alkyl.

In a class of this tenth embodiment, W is

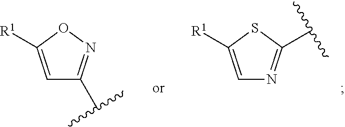

### and R¹ is

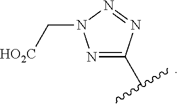

In an eleventh embodiment of the compounds of the present invention, Het is selected from the group consisting of:

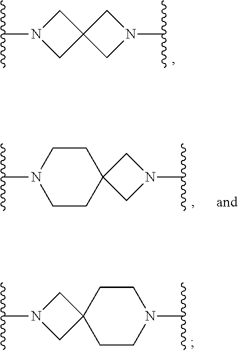

Ar is phenyl optionally substituted with one to three substituents each independently selected from methyl, halogen, trifluoromethyl, and trifluoromethoxy;
W is heteroaryl selected from the group consisting of:

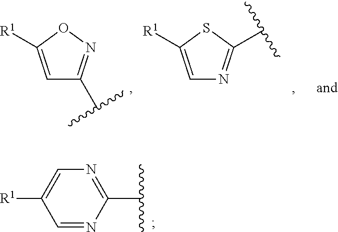

and R¹ is heteroaryl selected from the group consisting of:

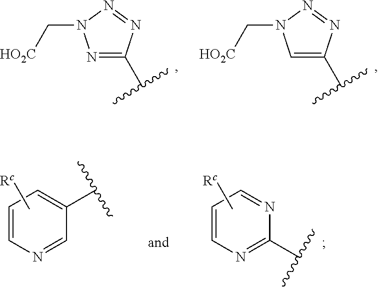

wherein R^{c} is —CO₂H, —CO₂C₁₋₃ alkyl, —CH₂CO₂H, or —CH₂CO₂C₁₋₃ alkyl.

In a class of this eleventh embodiment, Het is

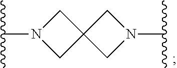

### W is

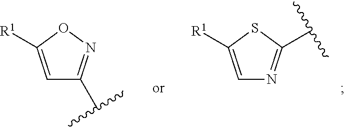

### and R¹ is

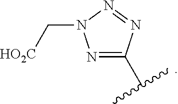

Illustrative, but nonlimiting, examples of compounds of the present invention that are useful as inhibitors of SCD are the following:

| Example | IC₅₀ hSCD-1 |
|---|---|
| 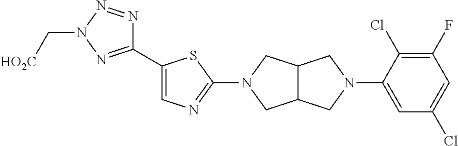 | 13 nM |
| 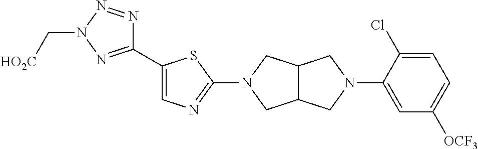 | 10 nM |
| 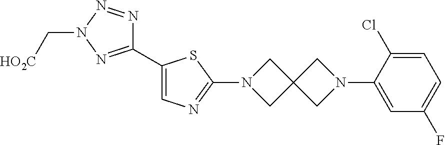 | 11 nM |
| 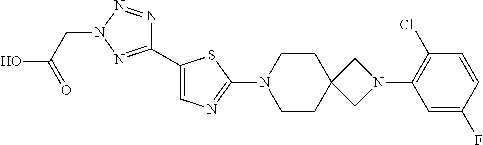 | 39 nM |
| 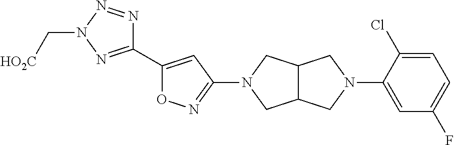 | 27 nM |
| 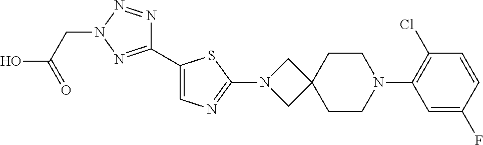 | 45 nM |
| 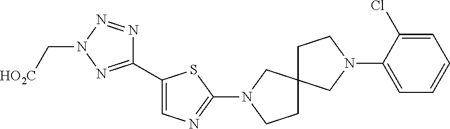 | 161 nM |
| 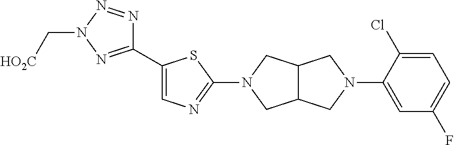 | 12 nM |
| 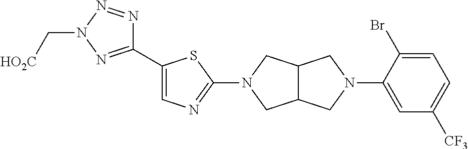 | 14 nM |
| 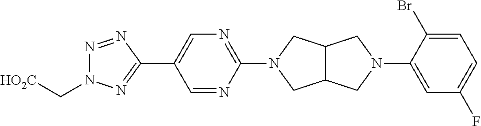 | 77 nM |
| 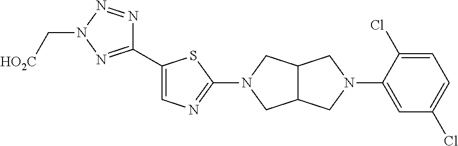 | 13 nM |

and pharmaceutically acceptable salts thereof.

For the purposes of clarity, the compounds of the present invention are further described by the structural formulae IIa-IIk:

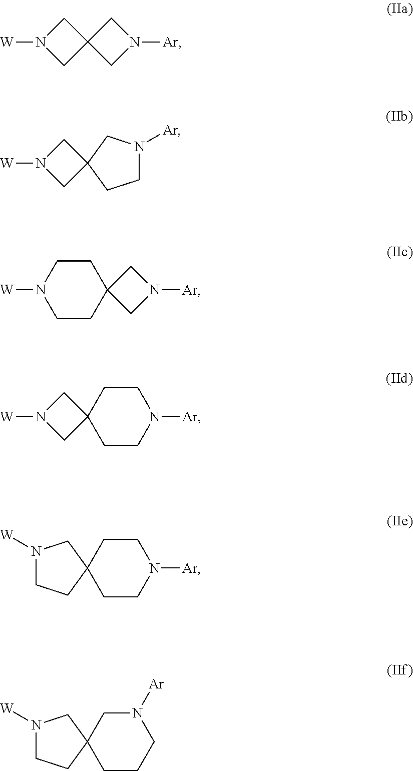

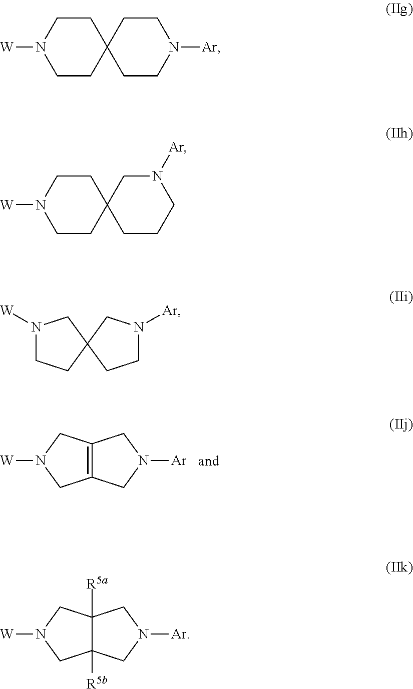

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃₋₁₀, the term alkyl also includes cycloalkyl groups, and combinations of linear or branched alkyl chains combined with cycloalkyl structures. When no number of carbon atoms is specified, C₁₋₆ is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeSO—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5(6H)-one-3-yl, and the like.

“Heteroaryl” means an aromatic or partially aromatic heterocycle that contains at least one ring heteroatom selected from O, S and N. Heteroaryls thus include heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

Compounds of structural formula I may contain one or more asymmetric centers and can thus occur as racemates and racemic mixtures, single enantiomers, diastereomeric mixtures and individual diastereomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural formula I are included in the present invention as well.

The subject compounds are useful in a method of inhibiting the stearoyl-coenzyme A delta-9 desaturase enzyme (SCD) in a patient such as a mammal in need of such inhibition comprising the administration of an effective amount of the compound. The compounds of the present invention are therefore useful to control, prevent, and/or treat conditions and diseases mediated by high or abnormal SCD enzyme activity.

Thus, one aspect of the present invention concerns a method of treating hyperglycemia, diabetes or insulin resistance in a mammalian patient in need of such treatment, which comprises administering to said patient an effective amount of a compound in accordance with structural formula I or a pharmaceutically salt or solvate thereof.

A second aspect of the present invention concerns a method of treating non-insulin dependent diabetes mellitus (Type 2 diabetes) in a mammalian patient in need of such treatment comprising administering to the patient an antidiabetic effective amount of a compound in accordance with structural formula I.

A third aspect of the present invention concerns a method of treating obesity in a mammalian patient in need of such treatment comprising administering to said patient a compound in accordance with structural formula I in an amount that is effective to treat obesity.

A fourth aspect of the invention concerns a method of treating metabolic syndrome and its sequelae in a mammalian patient in need of such treatment comprising administering to said patient a compound in accordance with structural formula I in an amount that is effective to treat metabolic syndrome and its sequelae. The sequelae of the metabolic syndrome include hypertension, elevated blood glucose levels, high triglycerides, and low levels of HDL cholesterol.

A fifth aspect of the invention concerns a method of treating a lipid disorder selected from the group consisting of dyslipidemia, hyperlipidemia, hypertriglyceridemia, hypercholesterolemia, low HDL and high LDL in a mammalian patient in need of such treatment comprising administering to said patient a compound in accordance with structural formula I in an amount that is effective to treat said lipid disorder.

A sixth aspect of the invention concerns a method of treating atherosclerosis in a mammalian patient in need of such treatment comprising administering to said patient a compound in accordance with structural formula I in an amount effective to treat atherosclerosis.

A seventh aspect of the invention concerns a method of treating cancer in a mammalian patient in need of such treatment comprising administering to said patient a compound in accordance with structural formula I in an amount effective to treat cancer. In one embodiment of this aspect of the invention, the cancer is liver cancer.

A further aspect of the invention concerns a method of treating a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) metabolic syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to treat said condition.

Yet a further aspect of the invention concerns a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) metabolic syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to delay the onset of said condition.

Yet a further aspect of the invention concerns a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) metabolic syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to reduce the risk of developing said condition.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, species can be treated. However, the method can also be practiced in other species, such as avian species (e.g., chickens).

The present invention is further directed to a method for the manufacture of a medicament for inhibiting stearoyl-coenzyme A delta-9 desaturase enzyme activity in humans and animals comprising combining a compound of the present invention with a pharmaceutically acceptable carrier or diluent. More particularly, the present invention is directed to the use of a compound of structural formula I in the manufacture of a medicament for use in treating a condition selected from the group consisting of hyperglycemia, Type 2 diabetes, insulin resistance, obesity, and a lipid disorder in a mammal, wherein the lipid disorder is selected from the group consisting of dyslipidemia, hyperlipidemia, hypertriglyceridemia, hypercholesterolemia, low HDL, and high LDL.

The subject treated in the present methods is generally a mammal, preferably a human being, male or female, in whom inhibition of stearoyl-coenzyme A delta-9 desaturase enzyme activity is desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and/or “administering a” compound should be understood to mean providing a compound of the invention or a prodrug of a compound of the invention to the individual in need of treatment.

The utility of the compounds in accordance with the present invention as inhibitors of stearoyl-coenzyme A delta-9 desaturase (SCD) enzyme activity may be demonstrated by the following microsomal and whole-cell based assays:

### I. SCD Enzyme Activity Assay:

The potency of compounds of formula I against the stearoyl-CoA desaturase was determined by measuring the conversion of radiolabeled stearoyl-CoA to oleoyl-CoA using rat liver microsome or human SCD1 (hSCD-1) following previously published procedures with some modifications (Joshi, et al., J. Lipid Res., 18: 32-36 (1977); Talamo, et al., Anal. Biochem, 29: 300-304 (1969)). Liver microsome was prepared from male Wistar or Sprague Dawley rats on a high carbohydrate diet for 3 days (LabDiet #5803, Purina). The livers were homogenized (1:10 w/v) in a buffer containing 250 mM sucrose, 1 mM EDTA, 5 mM DTT and 50 mM Tris-HCl (pH 7.5). After a 100,000×g centrifugation for 60 min, the liver microsome pellet was suspended in a buffer containing 100 mM sodium phosphate, 20% glycerol, 2 mM DTT, and stored at −78° C. Human SCD1 desaturase system was reconstituted using human SCD1 from a baculovirus/Sf9 expression system, cytochrome B5 and cytochrome B5 reductase. Typically, test compound in 2 μL DMSO was incubated for 15 min at room temperature with 180 μL of the SCD enzyme in a buffer containing 100 mM Tris-HCl (pH 7.5), ATP (5 mM), Coenzyme-A (0.1 mM), Triton X-100 (0.5 mM) and NADH (2 mM). The reaction was initiated by the addition of 20 μL of [³H]-stearoyl-CoA (final concentration=2 μM, radioactivity concentration=1 μCi/mL). After 10 min, the reaction mixture (80 μL) was mixed with a calcium chloride/charcoal aqueous suspension (100 μL charcoal (10% w/v) plus 25 μL CaCl₂ (2N). After centrifugation to precipitate the radioactive fatty acid species, tritiated water released from 9,10-[³H]-stearoyl-CoA by the SCD enzyme was quantified on a scintillation counter.

### II. Whole Cell-Based SCD (Delta-9), Delta-5 and Delta-6 Desaturase Assays:

Human HepG2 cells were grown on 96-well plates in MEM media (Gibco cat #11095-072) supplemented with 10% heat-inactivated fetal bovine serum at 37° C. under 5% CO₂ in a humidified incubator. Test compound dissolved in the media was incubated with the sub-confluent cells for 15 min at 37° C. [1-⁻¹⁴]-stearic acid was added to each well to a final concentration of 0.05 μCi/mL to detect SCD-catalyzed [¹⁴C]-oleic acid formation. 0.05 μCi/mL of [1-⁻¹⁴]-eicosatrienoic acid or [1-⁻¹⁴C]-linolenic acid plus 10 μM of 2-amino-N-(3-chlorophenyl)benzamide (a delta-5 desaturase inhibitor) was used to index the delta-5 and delta-6 desaturase activities, respectively. After 4 h incubation at 37° C., the culture media was removed and the labeled cells were washed with PBS (3×1 mL) at room temperature. The labeled cellular lipids were hydrolyzed under nitrogen at 65° C. for 1 h using 400 μL of 2N sodium hydroxide plus 50 μL of L-α-phosphatidylcholine (2 mg/mL in isopropanol, Sigma #P-3556). After acidification with phosphoric acid (60 μL), the radioactive species were extracted with 300 μL of acetonitrile and quantified on a HPLC that was equipped with a C-18 reverse phase column and a Packard Flow Scintillation Analyzer. The levels of [¹⁴C]-oleic acid over [¹⁴]-stearic acid, [¹⁴C]-arachidonic acid over [¹⁴C]-eicosatrienoic acid, and [¹⁴C]-eicosatetraenoic acid (8,11,14,17) over [¹⁴C]-linolenic acid were used as the corresponding activity indices of SCD, delta-5 and delta-6 desaturase, respectively.

The SCD inhibitors of structural formula I, particularly the compounds of the present invention denoted as non-limiting specific Examples below, exhibit an inhibition constant IC₅₀ of less than 1 μM, and more typically less than 0.1 μM, against the rat and human SCD enzymes. Generally, the IC₅₀ ratio for delta-5 or delta-6 desaturases to human or rat SCD for a compound of structural formula I, particularly for the specific Examples denoted below, is at least about ten or more, and preferably about one hundred or greater.

### In Vivo Efficacy of Compounds of the Present Invention:

The in vivo efficacy of compounds of formula I was determined by following the conversion of [1-¹⁴C]-stearic acid to [1-¹⁴C]oleic acid in animals as exemplified below. Mice were dosed with a compound of formula I and one hour later the radioactive tracer, [1-¹⁴C]-stearic acid, was dosed at 20 μCi/kg IV. At 3 h post dosing of the compound, the liver was harvested and then hydrolyzed in 10 N sodium hydroxide for 24 h at 80° C. After phosphoric acid acidification of the extract, the amount of stearic acid and [¹⁴C]-oleic acid was quantified on a HPLC system that was equipped with a C-18 reverse phase column and a Packard Flow Scintillation Analyzer

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred, particularly in combination with a pharmaceutically acceptable carrier. However, the combination therapy may also include therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Examples of other active ingredients that may be administered in combination with a compound of Formula I, and either administered separately or in the same pharmaceutical composition, include, but are not limited to:

(1) dipeptidyl peptidase-IV (DPP-4) inhibitors;

(2) insulin sensitizers, including (i) PPARγ agonists, such as the glitazones (e.g. pioglitazone, rosiglitazone, netoglitazone, rivoglitazone, and balaglitazone) and other PPAR ligands, including (1) PPARα/γ, dual agonists, such as muraglitazar, aleglitazar, sodelglitazar, and naveglitazar, (2) PPARα agonists, such as fenofibric acid derivatives (gemfibrozil, clofibrate, ciprofibrate, fenofibrate and bezafibrate), (3) selective PPARγ modulators (SPPARγM's), such as those disclosed in WO 02/060388, WO 02/08188, WO 2004/019869, WO 2004/020409, WO 2004/020408, and WO 2004/066963, and (4) PPARγ partial agonists; (ii) biguanides, such as metformin and its pharmaceutically acceptable salts, in particular, metformin hydrochloride, and extended-release formulations thereof, such as Glumetza®, Fortamet®, and GlucophageXR®;

(iii) protein tyrosine phosphatase-1B (PTP-1B) inhibitors;

(3) insulin and insulin analogs or derivatives, such as insulin lispro, insulin detemir, insulin glargine, insulin glulisine, and inhalable formulations of each thereof;

(4) leptin and leptin derivatives, agonists, and analogs, such as metreleptin;

(5) amylin; amylin analogs, such as davalintide; and amylin agonists, such as pramlintide;

(6) sulfonylurea and non-sulfonylurea insulin secretagogues, such as tolbutamide, glyburide, glipizide, glimepiride, mitiglinide, and meglitinides, such as nateglinide and repaglinide;

(7) α-glucosidase inhibitors (such as acarbose, voglibose and miglitol);

(8) glucagon receptor antagonists, such as those disclosed in WO 98/04528, WO 99/01423, WO 00/39088, and WO 00/69810;

(9) incretin mimetics, such as GLP-1, GLP-1 analogs, derivatives, and mimetics (See for example, WO 2008/011446, U.S. Pat. No. 5,545,618, U.S. Pat. No. 6,191,102, and U.S. Pat. No. 56,583,111); and GLP-1 receptor agonists, such as oxyntomodulin and its analogs and derivatives (See for example, WO 2003/022304, WO 2006/134340, WO 2007/100535), glucagon and its analogs and derivatives (See for example, WO 2008/101017), exenatide, liraglutide, taspoglutide, albiglutide, AVE0010, CJC-1134-PC, NN9535, LY2189265, LY2428757, and BIM-51077, including intranasal, transdermal, and once-weekly formulations thereof, such as exenatide QW;

(10) LDL cholesterol lowering agents such as (i) HMG-CoA reductase inhibitors (lovastatin, simvastatin, pravastatin, cerivastatin, fluvastatin, atorvastatin, pitavastatin, and rosuvastatin), (ii) bile acid sequestering agents (such as cholestyramine, colestimide, colesevelam hydrochloride, colestipol, and dialkylaminoalkyl derivatives of a cross-linked dextran, (iii) inhibitors of cholesterol absorption, such as ezetimibe, and (iv) acyl CoA:cholesterol acyltransferase inhibitors, such as avasimibe;

(11) HDL-raising drugs, such as niacin or a salt thereof and extended-release versions thereof; MK-524A, which is a combination of niacin extended-release and the DP-1 antagonist MK-524; and nicotinic acid receptor agonists;

(12) antiobesity compounds;

(13) agents intended for use in inflammatory conditions, such as aspirin, non-steroidal anti-inflammatory drugs (NSAIDs), glucocorticoids, and selective cyclooxygenase-2 (COX-2) inhibitors;

(14) antihypertensive agents, such as ACE inhibitors (such as enalapril, lisinopril, ramipril, captopril, quinapril, and tandolapril), A-II receptor blockers (such as losartan, candesartan, irbesartan, olmesartan medoxomil, valsartan, telmisartan, and eprosartan), renin inhibitors (such as aliskiren), beta blockers (such as and calcium channel blockers (such as;

(15) glucokinase activators (GKAs), such as LY2599506;

(16) inhibitors of 11β-hydroxysteroid dehydrogenase type 1, such as those disclosed in U.S. Pat. No. 6,730,690; WO 03/104207; and WO 04/058741;

(17) inhibitors of cholesteryl ester transfer protein (CETP), such as torcetrapib and MK-0859;

(18) inhibitors of fructose 1,6-bisphosphatase, such as those disclosed in U.S. Pat. Nos. 6,054,587; 6,110,903; 6,284,748; 6,399,782; and 6,489,476;

(19) inhibitors of acetyl CoA carboxylase-1 or 2 (ACC1 or ACC2);

(20) AMP-activated Protein Kinase (AMPK) activators;

(21) agonists of the G-protein-coupled receptors: GPR-109, GPR-116, GPR-119, and GPR-40;

(22) SSTR3 antagonists, such as those disclosed in WO 2009/011836;

(23) neuromedin U receptor 1 (NMUR1) and/or neuromedin U receptor 2 (NMUR2) agonists, such as those disclosed in WO2007/109135 and WO2009/042053, including, but not limited to, neuromedin U (NMU) and neuromedin S (NMS) and their analogs and derivatives;

(24) GPR-105 (P2YR14) antagonists, such as those disclosed in WO 2009/000087;

(25) inhibitors of glucose uptake, such as sodium-glucose transporter (SGLT) inhibitors and its various isoforms, such as SGLT-1; SGLT-2, such as dapagliflozin and remogliflozin; and SGLT-3;

(26) inhibitors of acyl coenzyme A: diacylglycerol acyltransferase 1 and 2 (DGAT-1 and DGAT-2);

(27) inhibitors of fatty acid synthase;

(28) inhibitors of acyl coenzyme A: monoacylglycerol acyltransferase 1 and 2 (MGAT-1 and MGAT-2);

(29) agonists of the TGR5 receptor (also known as GPBAR1, BG37, GPCR19, GPR131, and M-BAR);

(30) bromocriptine mesylate and rapid-release formulations thereof;

(31) histamine H3 receptor agonists; and

(32) α2-adrenergic or β3-adrenergic receptor agonists.

Dipeptidyl peptidase-IV (DPP-4) inhibitors that can be used in combination with compounds of Formula I include, but are not limited to, sitagliptin (disclosed in U.S. Pat. No. 6,699,871), vildagliptin, saxagliptin, alogliptin, denagliptin, carmegliptin, dutogliptin, melogliptin, linagliptin, and pharmaceutically acceptable salts thereof, and fixed-dose combinations of these compounds with metformin hydrochloride, pioglitazone, rosiglitazone, simvastatin, atorvastatin, or a sulfonylurea.

Other dipeptidyl peptidase-IV (DPP-4) inhibitors that can be used in combination with compounds of Formula I include, but are not limited to:
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-2-(2,5-difluorophenyl)tetrahydro)-5-(4,6-dihydropyrrolo[3,4-c]pyrazol-5(1H)-yl) tetrahydro-2H-pyran-3-amine;
(3R)-4-[(3R)-3-amino-4-(2,4,5-trifluorophenyl)butanoyl]-hexahydro-3-methyl-2H-1,4-diazepin-2-one;
4-[(3R)-3-amino-4-(2,5-difluorophenyl)butanoyl]hexahydro-1-methyl-2H-1,4-diazepin-2-one hydrochloride; and
(3R)-4-[(3R)-3-amino-4-(2,4,5-trifluorophenyl)butanoyl]-hexahydro-3-(2,2,2-trifluoroethyl)-2H-1,4-diazepin-2-one; and pharmaceutically acceptable salts thereof.

Antiobesity compounds that can be combined with compounds of Formula I include topiramate; zonisamide; naltrexone; phentermine; bupropion; the combination of bupropion and naltrexone; the combination of bupropion and zonisamide; the combination of topiramate and phentermine; fenfluramine; dexfenfluramine; sibutramine; lipase inhibitors, such as orlistat and cetilistat; melanocortin receptor agonists, in particular, melanocortin-4 receptor agonists; CCK-1 agonists; melanin-concentrating hormone (MCH) receptor antagonists; neuropeptide Y₁ or Y₅ antagonists (such as MK-0557); CB1 receptor inverse agonists and antagonists (such as rimonabant and taranabant); β₃ adrenergic receptor agonists; ghrelin antagonists; bombesin receptor agonists (such as bombesin receptor subtype-3 agonists); histamine H3 receptor inverse agonists; 5-hydroxytryptamine-2c (5-HT2c) agonists, such as lorcaserin; and inhibitors of fatty acid synthase (FAS). For a review of anti-obesity compounds that can be combined with compounds of the present invention, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); J. A. Fernandez-Lopez, et al., “Pharmacological Approaches for the Treatment of Obesity,” Drugs, 62: 915-944 (2002); and K. M. Gadde, et al., “Combination pharmaceutical therapies for obesity,” Exp. Opin. Pharmacother., 10: 921-925 (2009).

Glucagon receptor antagonists that can be used in combination with the compounds of Formula I include, but are not limited to:
N-[4-((1S)-1-{3-(3,5-dichlorophenyl)-5-[6-(trifluoromethoxy)-2-naphthyl]-1H-pyrazol-1-yl}ethyl)benzoyl]-[β-alanine;
N-[4-((1R)-1-{3-(3,5-dichlorophenyl)-5-[6-(trifluoromethoxy)-2-naphthyl]-1H-pyrazol-1-yl}ethyl)benzoyl]-β-alanine;
N-(4-{1-[3-(2,5-dichlorophenyl)-5-(6-methoxy-2-naphthyl)-1H-pyrazol-1-yl]ethyl}benzoyl)-β-alanine;
N-(4-{(1S)-1-[3-(3,5-dichlorophenyl)-5-(6-methoxy-2-naphthyl)-1H-pyrazol-1-yl]ethyl}benzoyl)-β-alanine;
N-(4-{(1S)-1-[(R)-(4-chlorophenyl)(7-fluoro-5-methyl-1H-indol-3-yl)methyl]butyl}benzoyl)-β-alanine; and
N-(4-{(1S)-1-[(4-chlorophenyl)(6-chloro-8-methylquinolin-4-yl)methyl]butyl}benzoyl)-β-alanine; and pharmaceutically acceptable salts thereof.

Agonists of the GPR-119 receptor that can be used in combination with the compounds of Formula I include, but are not limited to:
rac-cis 5-chloro-2-{4-[2-(2-{[5-(methylsulfonyl)pyridin-2-yl]oxy}ethyl)cyclopropyl]piperidin-1-yl}pyrimidine;
5-chloro-2-{4-[(1R,2S)-2-(2-{[5-(methylsulfonyl)pyridin-2-yl]oxy}ethyl)cyclopropyl]piperidin-1-yl}pyrimidine;
rac cis-5-chloro-2-[4-(2-{2-[4-(methylsulfonyl)phenoxy]ethyl}cyclopropyl)piperidin-1-yl]pyrimidine;
5-chloro-2-[4-((1S,2R)-2-{2-[4-(methylsulfonyl)phenoxy]ethyl}cyclopropyl)piperidin-1-yl]pyrimidine;
5-chloro-2-[4-((1R,2S)-2-{2-[4-(methylsulfonyl)phenoxy]ethyl}cyclopropyl)piperidin-1-yl]pyrimidine;
rac cis-5-chloro-2-[4-(2-{2-[3-(methylsulfonyl)phenoxy]ethyl}cyclopropyl)piperidin-1-yl]pyrimidine; and
rac cis-5-chloro-2-[4-(2-{2-[3-(5-methyl-1,3,4-oxadiazol-2-yl)phenoxy]ethyl}cyclopropyl)piperidin-1-yl]pyrimidine; and
   pharmaceutically acceptable salts thereof.

Selective PPARγ modulators (SPPARγM's) that can be used in combination with the compounds of Formula I include, but are not limited to:
(2S)-2-({6-chloro-3-[6-(4-chlorophenoxy)-2-propylpyridin-3-yl]-1,2-benzisoxazol-5-yl}oxy)propanoic acid;
(2S)-2-({6-chloro-3-[6-(4-fluorophenoxy)-2-propylpyridin-3-yl]-1,2-benzisoxazol-5-yl}oxy)propanoic acid;
(2S)-2-{[6-chloro-3-(6-phenoxy-2-propylpyridin-3-yl)-1,2-benzisoxazol-5-yl]oxy}propanoic acid;
(2R)-2-({6-chloro-3-[6-(4-chlorophenoxy)-2-propylpyridin-3-yl]-1,2-benzisoxazol-5-yl}oxy)propanoic acid;
(2R)-2-{3-[3-(4-methoxy)benzoyl-2-methyl-6-(trifluoromethoxy)-1H-indol-1-yl]phenoxy}butanoic acid;
(2S)-2-{3-[3-(4-methoxy)benzoyl-2-methyl-6-(trifluoromethoxy)-1H-indol-1-yl]phenoxy}butanoic acid;
2-{3-[3-(4-methoxy)benzoyl-2-methyl-6-(trifluoromethoxy)-1H-indol-1-yl]phenoxy}-2-methylpropanoic acid; and
(2R)-2-{3-[3-(4-chloro)benzoyl-2-methyl-6-(trifluoromethoxy)-1H-indol-1-yl]phenoxy}propanoic acid; and
   pharmaceutically acceptable salts and esters thereof.

Inhibitors of 11β-hydroxysteroid dehydrogenase type 1 that can be used in combination with the compounds of Formula I include, but are not limited to:
3-[1-(4-chlorophenyl)-trans-3-fluorocyclobutyl]-4,5-dicyclopropyl-r-4H-1,2,4-triazole;
3-[1-(4-chlorophenyl)-trans-3-fluorocyclobutyl]-4-cyclopropyl-5-(1-methylcyclopropyl)-r-4H-1,2,4-triazole;
3-[1-(4-chlorophenyl)-trans-3-fluorocyclobutyl]-4-methyl-5-[2-(trifluoromethoxy)phenyl]-r-4H-1,2,4-triazole;
3-[1-(4-chlorophenyl)cyclobutyl]-4-methyl-5-[2-(trifluoromethyl)phenyl]-4H-1,2,4-triazole;
3-{4-[3-(ethylsulfonyl)propyl]bicyclo [2.2.2]oct-1-yl}-4-methyl-5-[2-(trifluoromethyl)phenyl]-4H-1,2,4-triazole;
4-methyl-3-{4-[4-(methylsulfonyl)phenyl]bicyclo [2.2.2]oct-1-yl}-5-[2-(trifluoromethyl)phenyl]-4H-1,2,4-triazole;
3-(4-{4-methyl-5-[2-(trifluoromethyl)phenyl]-4H-1,2,4-triazol-3-yl}bicyclo [2.2.2]oct-1-yl)-5-(3,3,3-trifluoropropyl)-1,2,4-oxadiazole;
3-(4-{4-methyl-5-[2-(trifluoromethyl)phenyl]-4H-1,2,4-triazol-3-yl}bicyclo [2.2.2]oct-1-yl)-5-(3,3,3-trifluoroethyl)-1,2,4-oxadiazole;
5-(3,3-difluorocyclobutyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
5-(1-fluoro-1-methylethyl)-3-(4-{4-methyl-5-[2-(trifluoromethyl)phenyl]-4H-1,2,4-triazol-3-yl}bicyclo [2.2.2]oct-1-yl)-1,2,4-oxadiazole;
2-(1,1-difluoroethyl)-5-(4-{4-methyl-5-[2-(trifluoromethyl)phenyl]-4H-1,2,4-triazol-3-yl}bicyclo[2.2.2]oct-1-yl)-1,3,4-oxadiazole;
2-(3,3-difluorocyclobutyl)-5-(4-{4-methyl-5-[2-(trifluoromethyl)phenyl]-4H-1,2,4-triazol-3-yl}bicyclo[2.2.2]oct-1-yl)-1,3,4-oxadiazole; and
5-(1,1-difluoroethyl)-3-(4-{4-methyl-5-[2-(trifluoromethyl)phenyl]-4H-1,2,4-triazol-3-yl}bicyclo[2.2.2]oct-1-yl)-1,2,4-oxadiazole; and pharmaceutically acceptable salts thereof.

Somatostatin subtype receptor 3 (SSTR3) antagonists that can be used in combination with the compounds of Formula I include, but are not limited to:

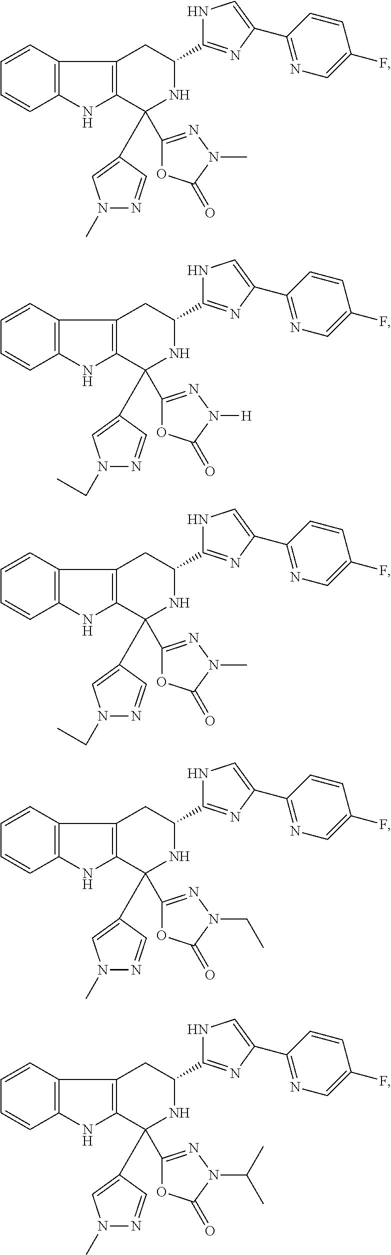

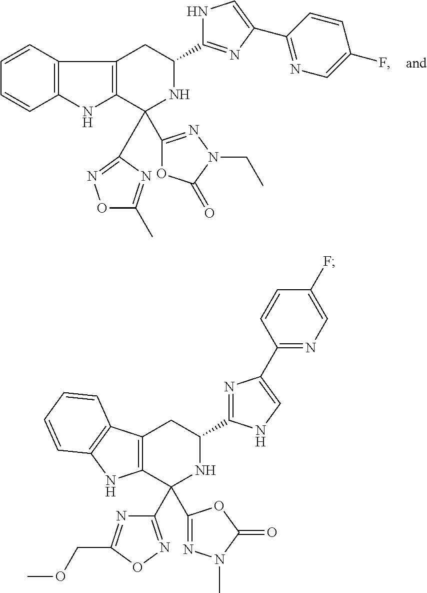

and pharmaceutically acceptable salts thereof.

AMP-activated Protein Kinase (AMPK) activators that can be used in combination with the compounds of Formula I include, but are not limited to:

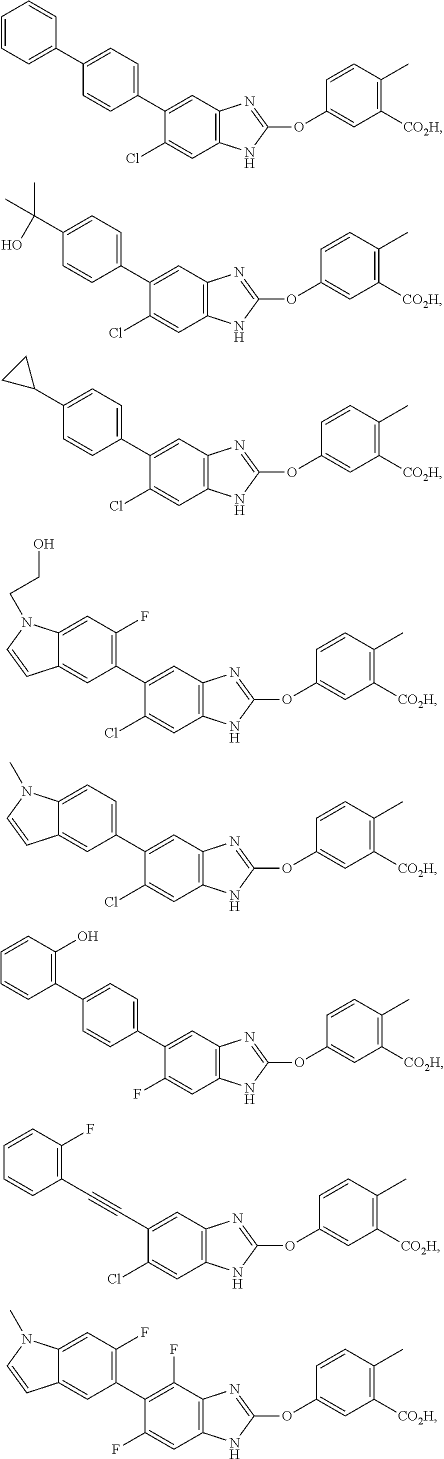

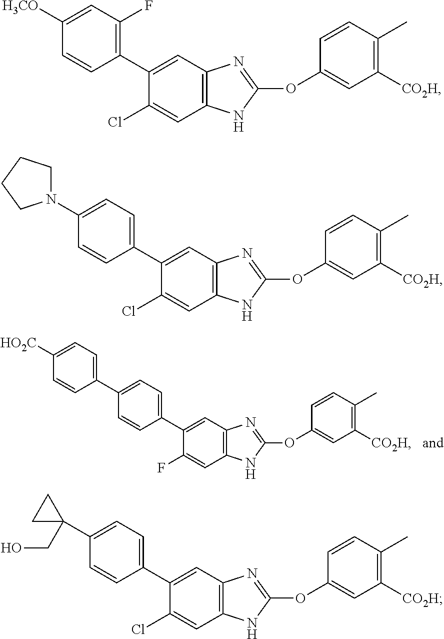

and pharmaceutically acceptable salts and esters thereof.

Inhibitors of acetyl-CoA carboxylase-1 and 2 (ACC-1 and ACC-2) that can be used in combination with the compounds of Formula I include, but are not limited to:
3-{1′-[(1-cyclopropyl-4-methoxy-1H-indol-6-yl)carbonyl]-4-oxospiro[chroman-2,4′-piperidin]-6-yl}benzoic acid;
5-{1′-[(1-cyclopropyl-4-methoxy-1H-indol-6-yl)carbonyl]-4-oxospiro[chroman-2,4′-piperidin]-6-yl}nicotinic acid;
1′-[(1-cyclopropyl-4-methoxy-1H-indol-6-yl)carbonyl]-6-(1H-tetrazol-5-yl)spiro[chroman-2,4′-piperidin]-4-one;
1′-[(1-cyclopropyl-4-ethoxy-3-methyl-1H-indol-6-yl)carbonyl]-6-(1H-tetrazol-5-yl)spiro[chroman-2,4′-piperidin]-4-one;
5-{1′-[(1-cyclopropyl-4-methoxy-3-methyl-1H-indol-6-yl)carbonyl]-4-oxo-spiro[chroman-2,4′-piperidin]-6-yl}nicotinic acid;
4′-({6-(5-carbamoylpyridin-2-yl)-4-oxospiro[chroman-2,4′-piperidin]-1′-yl}carbonyl)-2′,6′-diethoxybiphenyl-4-carboxylic acid;
2′,6′-diethoxy-4′-{[6-(1-methyl-1H-pyrazol-4-yl)-4-oxospiro[chroman-2,4′-piperidin]-1′-yl]carbonyl}biphenyl-4-carboxylic acid;
2′,6′-diethoxy-3-fluoro-4′-{[6-(1-methyl-1H-pyrazol-4-yl)-4-oxospiro[chroman-2,4′-piperidin]-1′-yl]carbonyl}biphenyl-4-carboxylic acid;
5-[4-({6-(3-carbamoylphenyl)-4-oxospiro[chroman-2,4′-piperidin]-1′-yl}carbonyl)-2,6-diethoxyphenyl]nicotinic acid;
sodium 4′-({6-(5-carbamoylpyridin-2-yl)-4-oxospiro[chroman-2,4′-piperidin]-1′-yl}carbonyl)-2′,6′-diethoxybiphenyl-4-carboxylate;
methyl 4′-({6-(5-carbamoylpyridin-2-yl)-4-oxospiro[chroman-2,4′-piperidin]-1′-yl}carbonyl)-2′,6′-diethoxybiphenyl-4-carboxylate;
1′-[(4,8-dimethoxyquinolin-2-yl)carbonyl]-6-(1H-tetrazol-5-yl)spiro[chroman-2,4′-piperidin]-4-one;
(5-{1′-[(4,8-dimethoxyquinolin-2-yl)carbonyl]-4-oxospiro[chroman-2,4′-piperidin]-6-yl}-2H-tetrazol-2-yl)methyl pivalate;
5-{1′-[(8-cyclopropyl-4-methoxyquinolin-2-yl)carbonyl]-4-oxospiro[chroman-2,4′-piperidin]-6-yl}nicotinic acid;
1′-(8-methoxy-4-morpholin-4-yl-2-naphthoyl)-6-(1H-tetrazol-5-yl)spiro[chroman-2,4′-piperidin]-4-one; and
1′-[(4-ethoxy-8-ethylquinolin-2-yl)carbonyl]-6-(1H-tetrazol-5-yl)spiro[chroman-2,4′-piperidin]-4-one; and
   pharmaceutically acceptable salts and esters thereof.

One particular aspect of combination therapy concerns a method of treating a condition selected from the group consisting of hypercholesterolemia, atherosclerosis, low HDL levels, high LDL levels, hyperlipidemia, hypertriglyceridemia, and dyslipidemia, in a mammalian patient in need of such treatment comprising administering to the patient a therapeutically effective amount of a compound of structural formula I and an HMG-CoA reductase inhibitor.

More particularly, this aspect of combination therapy concerns a method of treating a condition selected from the group consisting of hypercholesterolemia, atherosclerosis, low HDL levels, high LDL levels, hyperlipidemia, hypertriglyceridemia and dyslipidemia in a mammalian patient in need of such treatment wherein the HMG-CoA reductase inhibitor is a statin selected from the group consisting of lovastatin, simvastatin, pravastatin, cerivastatin, fluvastatin, atorvastatin, and rosuvastatin.

In another aspect of the invention, a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions is disclosed comprising administering to a mammalian patient in need of such treatment a therapeutically effective amount of a compound of structural formula I and an HMG-CoA reductase inhibitor.

In another aspect of the invention, a method for delaying the onset or reducing the risk of developing atherosclerosis in a human patient in need of such treatment is disclosed comprising administering to said patient an effective amount of a compound of structural formula I and an HMG-CoA reductase inhibitor.

More particularly, a method for delaying the onset or reducing the risk of developing atherosclerosis in a human patient in need of such treatment is disclosed, wherein the HMG-CoA reductase inhibitor is a statin selected from the group consisting of: lovastatin, simvastatin, pravastatin, cerivastatin, fluvastatin, atorvastatin, and rosuvastatin.

In another aspect of the invention, a method for delaying the onset or reducing the risk of developing atherosclerosis in a human patient in need of such treatment is disclosed,

wherein the HMG-Co A reductase inhibitor is a statin and further comprising administering a cholesterol absorption inhibitor.

More particularly, in another aspect of the invention, a method for delaying the onset or reducing the risk of developing atherosclerosis in a human patient in need of such treatment is disclosed, wherein the HMG-Co A reductase inhibitor is a statin and the cholesterol absorption inhibitor is ezetimibe.

In another aspect of the invention, a pharmaceutical composition is disclosed which comprises:

(1) a compound of structural formula I;
(2) a compound selected from the group consisting of:

(a) dipeptidyl peptidase IV (DPP-IV) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA: cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib; and

(s) inhibitors of fructose 1,6-bisphosphatase, such as those disclosed in U.S. Pat. Nos. 6,054,587; 6,110,903; 6,284,748; 6,399,782; and 6,489,476; and

(3) a pharmaceutically acceptable carrier.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations a compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require inhibition of stearoyl-CoA delta-9 desaturase enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing diabetes mellitus and/or hyperglycemia or hypertriglyceridemia or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Preparation of Compounds of the Invention

Synthetic methods for preparing the compounds of the present invention are illustrated in the following Schemes, Methods, and Examples. Starting materials are commercially available or may be prepared according to procedures known in the art or as illustrated herein. The compounds of the invention are illustrated by means of the specific examples shown below. However, these specific examples are not to be construed as forming the only genus that is considered as the invention. These examples further illustrate details for the preparation of the compounds of the present invention. Those skilled in the art will readily understand that known variations of the conditions and processes of the following preparative procedures can be used to prepare these compounds. All temperatures are in degrees Celsius unless otherwise noted. Mass spectra (MS) were measured by electrospray ion-mass spectroscopy (ESI). NMR spectra were recorded on Bruker instruments at 400 or 500 MHz.

### LIST OF ABBREVIATIONS

Alk=alkyl
Ar=aryl
BINAP=2,2′-bis(diphenylphosphino)-1,1′-binaphthalene
Boc=tert-butoxycarbonyl
br=broad
CH₂Cl₂=dichloromethane
d=doublet
DBU=1,8-diazabicyclo[5.4.0]undec-7-ene
DEAD=diethyl azodicarboxylate
DIPEA=N,N-diisopropylethylamine
DMF=dimethylformamide
DMSO=dimethyl sulfoxide
ESI=electrospray ionization
EtOAc=ethyl acetate
h=hours
HATU=O-(7-azabenzotriazol-1-yl)-N,N,N′,N′-tetramethyluronium hexafluorophosphate
HOAc=acetic acid
LiOH=lithium hydroxide
m=multiplet
MeCN=acetonitrile
MeOH=methyl alcohol
MeTHF=2-methyltetrahydrofuran
MgSO₄=magnesium sulfate
min=minutes
MS=mass spectroscopy
MTBE=methyl tert-butyl ether
NaOH=sodium hydroxide
Na₂SO₄=sodium sulfate
NMP=N-methyl 2-pyrrolidinone
NMR=nuclear magnetic resonance spectroscopy
PG=protecting group
Ph=phenyl
rt=room temperature
s=singlet
t=triplet
TFA=trifluoroacetic acid
TFAA=trifluoroacetic anhydride
THF=tetrahydrofuran
TMEDA=N,N,N′,N′-tetramethylethylenediamine

### Method A:

An appropriately substituted heteroaryl bromide 1 is reacted with concentrated ammonium hydroxide in a solvent such as THF to give amide 2. Dehydration with TFAA in a solvent such as CH₂Cl₂ gives the nitrile intermediate 3. The nitrile intermediate 3 is reacted with NaN₃ in the presence of a Lewis acid catalyst such as ZnBr₂ or a protic acid such as NH₄Cl and a solvent such as 2-propanol. The tetrazole 4 is then reacted with ethyl bromoacetate in the presence of a base such as Et₃N or an alkali metal (K, Na, Cs) carbonate in a solvent such as THF, 1,4-dioxane or DMF at a temperature range of room temperature to 150° C. The 2-alkylated ester tetrazole 5 is obtained together with the 1-alkylated isomer 6, which can be separated by chromatography.

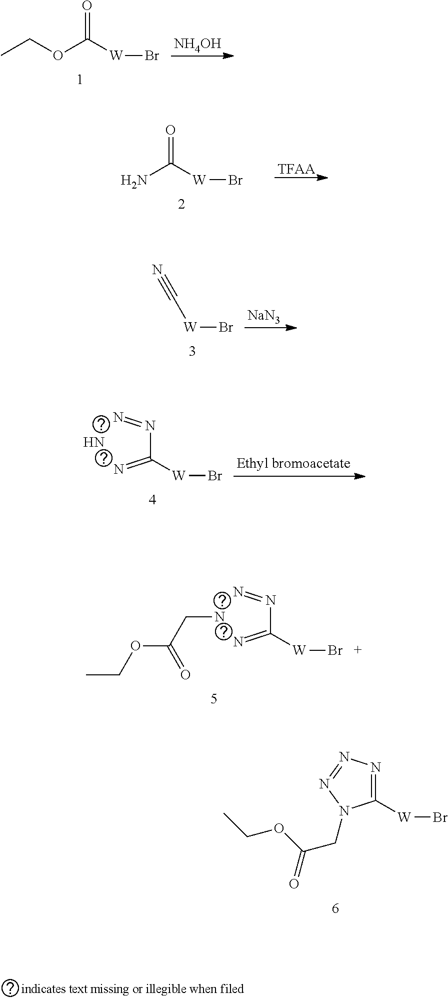

### Method B:

Alternatively, the tetrazole intermediate 4 can also be reacted with t-butyl bromoacetate in the presence of a base such as Et₃N or an alkali metal (K, Na, Cs) carbonate in a solvent such as THF, 1,4-dioxane or DMF at a temperature range of room temperature to 150° C. The 2-alkylated ester tetrazole 7 is normally obtained together with the 1-alkylated isomer 8, which can be separated by chromatography.

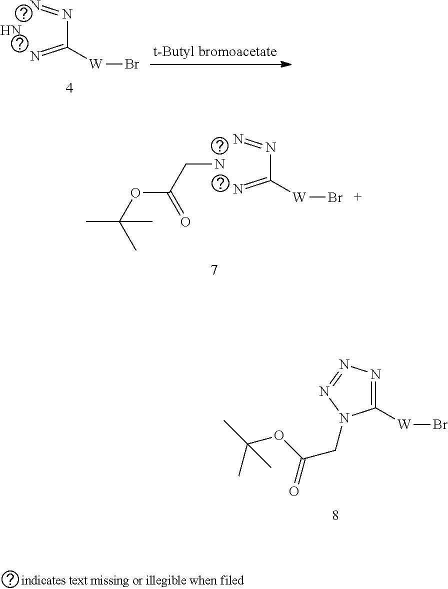

### Method C:

The intermediate 5 is reacted with tert-butyl hexahydropyrrolo[3,4-c]pyrrole-2(1H)-carboxylate with a base such as Et₃N or an alkali metal (Na, K) carbonate in a solvent such as THF, 1,4-dioxane, DMF, or NMP at a temperature range of room temperature to 150° C. to give 9. The Boc group is cleaved in the presence of a protic acid such as HCl in a solvent such as THF or 1,4-dioxane to give intermediate 10. The intermediate 10 is reacted under aryl amination conditions with the appropriately substituted aryl bromide or aryl iodide in the presence of a ligand such as BINAP, a catalyst such as palladium(II) acetate, a base such as an alkali metal (Na, K, Cs) carbonate or tert-butoxide and a solvent such as toluene at a temperature range of room temperature to 120° C. to give the ester precursor of intermediate 11. The carboxylic acid 11 is obtained by reacting the ester precursor with an alkali metal (Li, Na, K) hydroxide in a solvent system such as THF—H₂O and/or MeOH—H₂O.

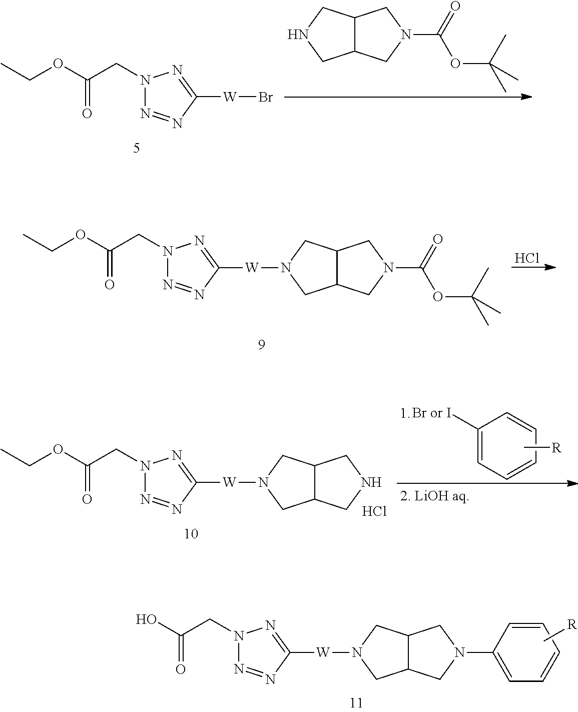

### Method D:

A mono N-tert-butoxycarbonyl protected diazabicycloalkane 12, or a salt thereof, is reacted under aryl amination conditions with the appropriately substituted aryl bromide or aryl iodide in the presence of a ligand such as BINAP, a catalyst such as palladium(II) acetate, a base such as an alkali metal (Na, K, Cs) carbonate or tert-butoxide and a solvent such as toluene at a temperature range of room temperature to 120° C. to give the intermediate 13. The Boc protecting group is cleaved in the presence of a protic acid such as HCl in a solvent such as THF or 1,4-dioxane to give intermediate 14. The intermediate 14 is reacted with the intermediate 5 in the presence of a base such as Et₃N or an alkali metal (Na, K) carbonate in a solvent such as THF, dioxane, DMF or NMP at a temperature range of room temperature to 150° C. to give the ester precursor of intermediate 11. The carboxylic acid 11 is obtained by reacting the ester precursor with an alkali metal (Li, Na, K) hydroxide in a solvent system such as THF—H₂O and/or MeOH—H₂O.

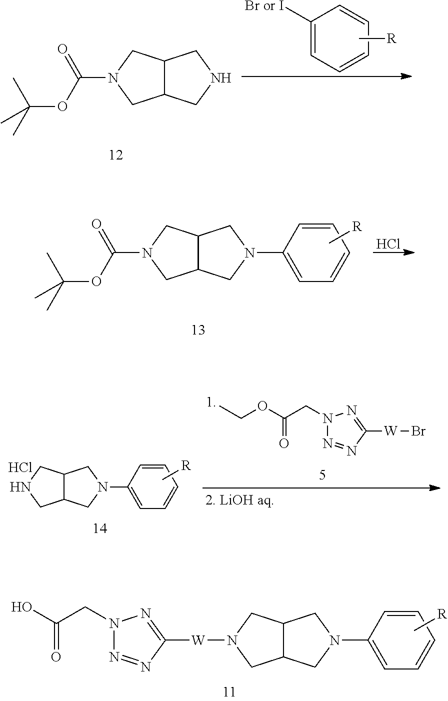

### Method E:

A mono N-tert-butoxycarbonyl protected diazaspiroalkane 15, or a salt thereof, is reacted under aryl amination conditions with the appropriately substituted aryl bromide or aryl iodide in the presence of a ligand such as BINAP, a catalyst such as palladium(II) acetate, a base such as an alkali metal (Na, K, Cs) carbonate or tert-butoxide and a solvent such as toluene at a temperature range of room temperature to 120° C. to give the intermediate 16. The Boc protecting group is cleaved in the presence of a protic acid such as HCl in a solvent such as THF or 1,4-dioxane to give intermediate 17. The intermediate 17 is reacted with intermediate 7 in the presence of a base such as Et₃N or an alkali metal (Na, K) carbonate in a solvent such as THF, dioxane, DMF or NMP at a temperature range of room temperature to 150° C. to give the tert-butyl ester precursor of intermediate 18. The carboxylic acid 18 is obtained by submitting the tert-butyl ester precursor to acidic conditions such as neat formic acid or TFA in a solvent such as CH₂Cl₂.

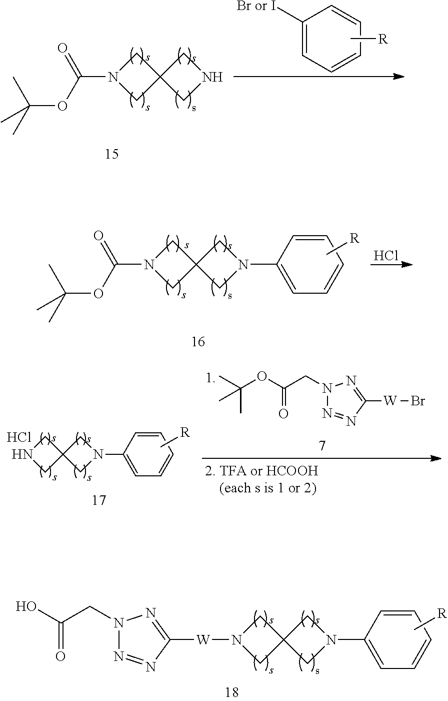

### Method F:

Where W represents an isoxazole residue, a mixture of the oxime 19 and an acrylate 20 are reacted at a temperature range of −78° C. to room temperature in the presence of a base such as alkali metal (Na, K) bicarbonate in a solvent system such as THF, DMF, DMF-H₂O to give the intermediate 21. The ester 21 is converted into the primary amide 22 according to the steps described in Method A. The intermediate 22 is reacted with an N-aryl diazabicycloalkane such as intermediate 14 in the presence of a base such an alkali metal (Na, K, Cs) carbonate in an alcoholic solvent such as EtOH or tert-butanol at a temperature range of room temperature to 150° C. to give the intermediate 23. The intermediate 24 is obtained by oxidation with iodine in the presence of imidazole. Intermediate 24 is reacted following suitable steps described in Method A to give intermediate 25. Alkylation of tetrazole 25 to give intermediate 26 is achieved following the step described in Method B. The carboxylic acid 27 is obtained following the suitable steps outlined in Method E.

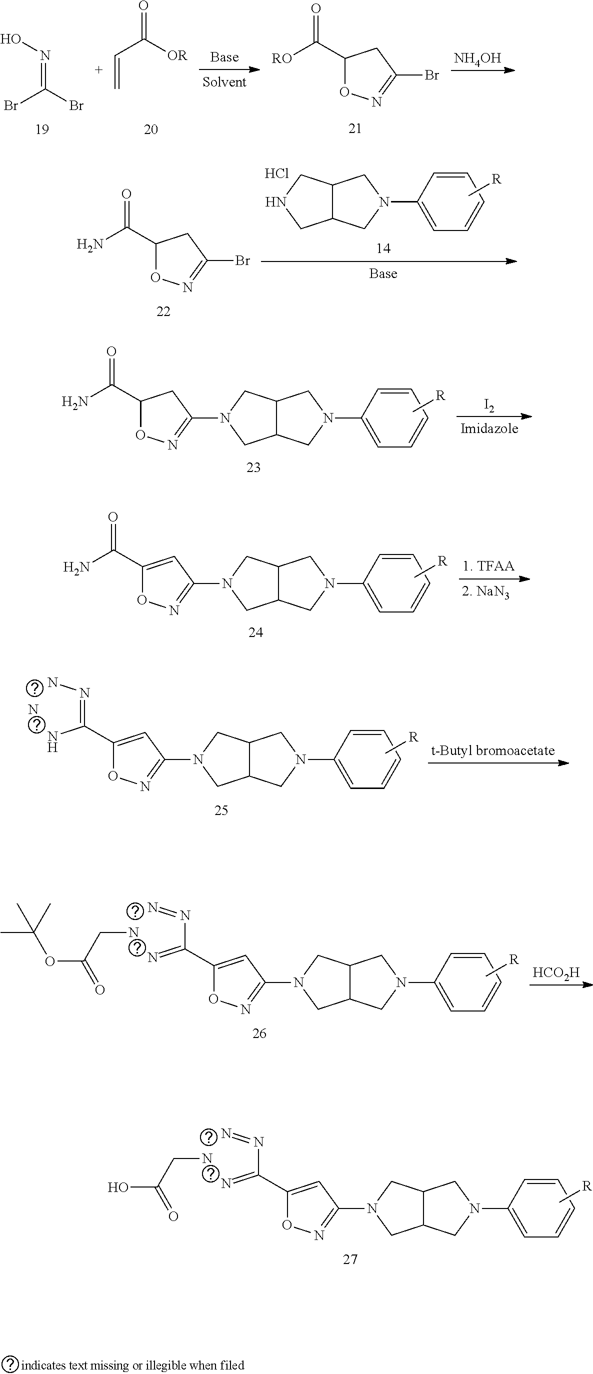

### Method G:

The intermediate 22 is dehydrated and tetrazole 28 is obtained following suitable steps outlined in Method A. Alkylation of the tetrazole 28 to give intermediate 29 is also conducted according to Method B. The intermediate 29 is reacted with intermediate 14 in a similar fashion to Method F. Oxidation of intermediate 30 is also conducted according to reaction conditions described in Method F to give the intermediate 26, common to Method F and Method G.

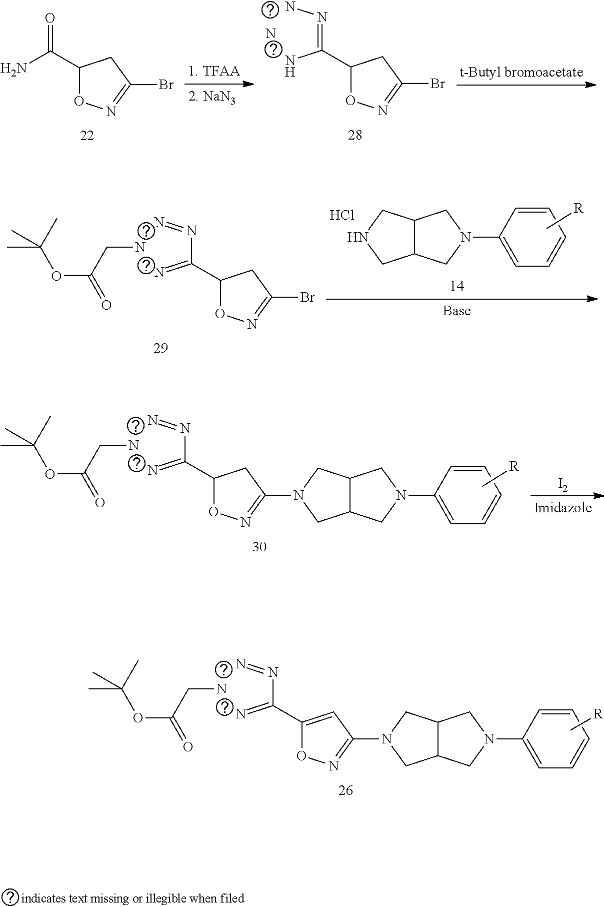

### Preparation of Key Intermediates

The following intermediates were purchased from commercial suppliers:

(a) tert-butyl hexahydropyrrolo[3,4-c]pyrrole-2(1H)-carboxylate hydrochloride:

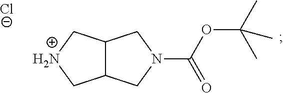

(b) 3,3-bis(bromomethyl)oxetane:

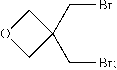

(c) 2-(tert-butoxycarbonyl)-2,7-diazaspiro[3.5]nonane:

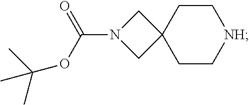

and (d) 7-(tert-butoxycarbonyl)-2,7-diazaspiro[3.5]nonane:

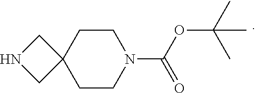

Procedures for the synthesis of tert-butyl hexahydropyrrolo[3,4-c]pyrrole-2(1H)-carboxylate are described in the literature, such as patent application publications US 2005/101602 (12 May 2005) and WO 2002/060902 (8 Aug. 2002). The synthesis of related pyrrolo[3,4-c]pyrroles, including 1,2,3,4,5,6-hexahydropyrrolo[3,4-c]pyrrole and 1,2,3,5-tetrahydropyrrolo[3,4-c]pyrrole, have also been described in the literature, as in Jendralla, H.; Fischer, G., Heterocycles 1995, 41, 1291-1298.

Other intermediates were prepared as follows:

### Intermediate 1

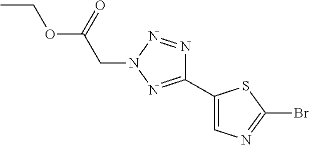

### Ethyl [5-(2-bromo-1,3-thiazol-5-yl)-2H-tetrazol-2-yl]acetate

### Step 1: 2-Bromo-1,3-thiazole-5-carboxamide

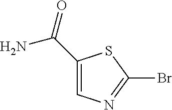

Into a 2 L round-bottom flask was added ethyl 2-bromothiazole-5-carboxylate (50.0 g, 212 mmol), THF (500 mL) and MeOH (250 mL). To this was added concentrated ammonium hydroxide in water (590 mL) and the reaction mixture was stirred at room temperature for 4 h. The solvents were removed under reduced pressure and the crude mixture poured into a separatory funnel containing brine (1 L). The aqueous layer was extracted with EtOAc (4×500 mL) and the combined organic layers were washed with brine, dried over Na₂SO₄, filtered and concentrated under reduced pressure.

### Step 2: 2-Bromo-1,3-thiazole-5-carbonitrile

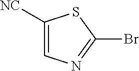

Into a 2 L round-bottom flask containing 2-bromo-1,3-thiazole-5-carboxamide (41.5 g, 201 mmol) in CH₂Cl₂ (1.3 L) was added triethylamine (70 mL, 502 mmol). The resulting solution was cooled to 0° C. and TFAA (34 mL, 241 mmol) was added slowly over 15 min. The reaction mixture was allowed to warm to room temperature and stirred for 2 h. The reaction mixture was poured into a 3 L separatory funnel containing saturated aqueous NaHCO₃ solution (500 mL). The aqueous layer was extracted with CH₂Cl₂ (2×1.2 L) and the combined organic layers were washed with brine, dried over Na₂SO₄, filtered and concentrated under reduced pressure. The crude reaction mixture was filtered through a short plug of silica gel on a sintered glass funnel, washing with copious quantities of EtOAc. The filtrate was concentrated under reduced pressure to provide the title compound.

### Step 3: 5-(2-Bromo-1,3-thiazol-5-yl)-2H-tetrazole

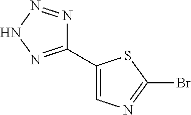

A solution of 2-bromo-1,3-thiazole-5-carbonitrile (5.00 g, 26.5 mmol) in 2-propanol (75 mL) and water (38 mL) was treated with ZnBr₂ (5.96 g, 26.5 mmol) and sodium azide (2.58 g, 39.7 mmol). The reaction mixture was heated at 120° C. for 5 h. The cooled reaction mixture was diluted with water (50 mL) and acidified to pH 3 using aqueous 1 N HCl solution (about 20 mL). The mixture was poured into a 500 mL separatory funnel and the aqueous layer was extracted with EtOAc (4×100 mL). The combined organic layers were washed with brine, dried over Na₂SO₄, filtered and concentrated under reduced pressure to provide the tetrazole compound.

### Step 4: Ethyl [5-(2-bromo-1,3-thiazol-5-O-2H-tetrazol-2-yl]acetate

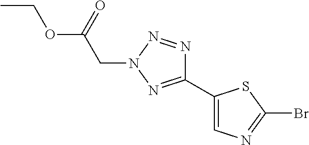

Into a 250 mL round-bottom flask containing 5-(2-bromo-1,3-thiazol-5-yl)-2H-tetrazole (5.43 g, 22.5 mmol) in THF (81 mL) was added triethylamine (7.2 mL, 52 mmol) and ethyl bromoacetate (3.8 mL, 34 mmol). The resulting mixture was heated at 80° C. for 1 h, and then cooled to room temperature. The reaction mixture was poured into a separatory funnel containing water (80 mL) and the aqueous layer was extracted with EtOAc (2×160 mL). The combined organic layers were washed with brine, dried over Na₂SO₄, filtered and concentrated under reduced pressure. Purification by column chromatography on silica gel, eluting with 100% hexanes to 50:50 hexanes:EtOAc as a gradient provided the desired alkylated tetrazole as a single regioisomer.

¹H NMR (d₆-DMSO, 400 MHz): δ 8.39 (1H, s), 5.93 (2H, s), 4.21 (2H, q, J=7.0 Hz), 1.22 (3H, t, J=7.0 Hz).

### Intermediate 2

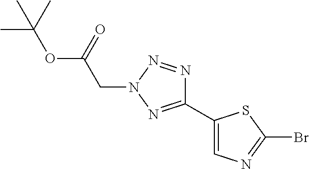

### tert-Butyl [5-(2-bromo-1,3-thiazol-5-yl)-2H-tetrazol-2-yl]acetate

This compound was synthesized in a similar manner as ethyl [5-(2-bromo-1,3-thiazol-5-yl)-2H-tetrazol-2-yl]acetate (Intermediate 1) using tert-butyl bromoacetate in place of ethyl bromoacetate.

¹H NMR (CDCl₃, 400 MHz): δ 8.22 (1H, s), 5.32 (2H, s), 1.47 (9H, s).

MS (ESI, Q⁺) m/z 346, 348 (M+1, ⁷⁹Br, ⁸¹Br).

### Intermediate 3

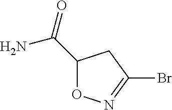

### 3-Bromo-4,5-dihydroisoxazole-5-carboxamide

### Step 1: Ethyl 3-bromo-4,5-dihydroisoxazole-5-carboxylate

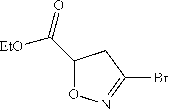

To a round-bottom flask containing hydroxycarbonimidic dibromide (100 g, 490 mmol) was slowly added DMF (300 mL) followed by ethyl acrylate (59 g, 590 mmol). The mixture was cooled to −10° C. and then a solution of KHCO₃ (99 g, 990 mmol) in water (400 mL) was added dropwise over 90 min, at a rate which maintained the internal temperature below 0° C. Stirring was continued at 0° C. for 1.5 h. The reaction mixture was poured into a 4 L separatory funnel containing water (500 mL) and the aqueous layer was extracted with MTBE (3×500 mL). The combined organic layers were washed with brine, dried over MgSO₄, filtered and concentrated under reduced pressure to give a yellow oil which was used directly in Step 2.

### Step 2: 3-Bromo-4,5-dihydroisoxazole-5-carboxamide

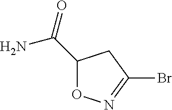

Ethyl 3-bromo-4,5-dihydroisoxazole-5-carboxylate (109 g, 490 mmol) was added to a 1 L round-bottom flask containing 2.0 M NH₃ in MeOH (295 mL). The reaction mixture was heated at 50° C. for 2.5 h and then cooled to room temperature and stirred overnight for 16 h. The resulting slurry was diluted with 500 mL of diethyl ether and stirred in an ice-bath for 1 h. The product was isolated by filtration under vacuum, affording the title compound as a tan solid.

¹H NMR (CDCl₃, 400 MHz): δ 6.70 (1H, bs), 5.92 (1H, bs), 5.06 (1′-1, dd, J=11.0, 6.5 Hz), 3.64-3.51 (2H, m). MS (ESI, Q⁺) m/z 193, 195 (M+1, ⁷⁹Br, ⁸¹Br).

### Intermediate 4

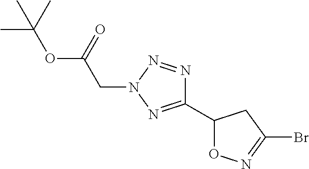

### tert-Butyl [5-(3-bromo-4,5-dihydroisoxazol-5-yl)-2H-tetrazol-2-yl]acetate

### Step 1: 3-Bromo-4,5-dihydroisoxazole-5-carbonitrile

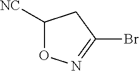

To a solution of 3-bromo-4,5-dihydroisoxazole-5-carboxamide (Intermediate 3, 30.0 g, 155 mmol) in THF (360 mL) was added triethylamine (43.0 mL, 311 mmol). The solution was cooled to 0° C. and TFAA (33.0 mL, 233 mmol) was added slowly over 20 min, at a rate which maintained the internal temperature below 15° C. The reaction mixture was stirred at 0° C. for 1 h. The reaction mixture was poured into a 2 L separatory funnel containing water (500 mL) and the aqueous layer was extracted with MTBE (3×500 mL). The combined organic layers were washed with a saturated aqueous NaHCO₃ solution (2×250 mL), brine, dried over MgSO₄, filtered and concentrated under reduced pressure to afford the title compound.

### Step 2: 5-(3-Bromo-4,5-dihydroisoxazol-5-yl)-2H-tetrazole

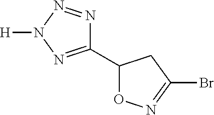

Into a 2 L round-bottom flask equipped with a reflux condenser, heating mantle and under N₂ was added 3-bromo-4,5-dihydroisoxazole-5-carbonitrile (39.4 g, 225 mmol), zinc oxide (1.8 g, 23 mmol), THF (40 mL) and water (200 mL). To this solution was slowly added a solution of sodium azide (16 g, 250 mmol) in water (10 mL) over 5 min and the mixture was warmed to 75° C. for 16 h. Heating was applied at a rate such that the internal temperature of the reaction mixture did not exceed 80° C. The reaction mixture was cooled to 0° C. and acidified to pH 3-4 with slow addition of 2 N aqueous HCl solution. During the acidification, the internal temperature was maintained below 5° C. The reaction mixture was poured into a 2 L separatory funnel and the aqueous layer was extracted with EtOAc (3×500 mL). The combined organic layers were washed with brine, dried over MgSO₄, filtered and concentrated under reduced pressure to afford the title compound.

### Step 3: tert-Butyl [5-(3-bromo-4,5-dihydroisoxazol-5-yl)-2H-tetrazol-2-yl]acetate

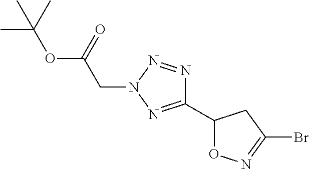

Into a 2 L round-bottom flask equipped with a reflux condenser, heating mantle and under N₂ was added 5-(3-bromo-4,5-dihydroisoxazol-5-yl)-2H-tetrazole (49 g, 225 mmol) and THF (500 mL). Triethylamine (53 mL, 383 mmol) was added to the mixture which was then heated to 55° C. while tert-butyl bromoacetate (66 g, 338 mmol) was added. The mixture was heated at 55° C. for 1 h and then cooled to room temperature. The reaction mixture was poured into a 2 L separatory funnel containing 1 N aqueous HCl solution (500 mL) and the aqueous layer was extracted with EtOAc (3×500 mL). The combined organic layers were washed with brine, dried over MgSO₄, filtered and concentrated under reduced pressure. Purification by column chromatography through iatrobead silica gel, eluting with 75:15:5 hexanes:EtOAc:CH₂Cl₂, afforded the title product in a greater than 10:1 regioisomeric purity.

¹H NMR (CDCl₃, 400 MHz): δ 5.98 (1H, dd, J=11.0, 7.5 Hz), 5.35 (2H, s), 3.87 (1H, dd, J=17.5, 7.5 Hz), 3.70 (1H, dd, J=17.5, 11.0 Hz), 1.50 (9H, s).

MS (ESI, Q⁺) m/z 332, 334 (M+1, ⁷⁹Br, ⁸¹Br).

### Intermediate 5

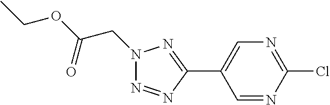

### Ethyl [5-(2-chloropyrimidin-5-yl)-2-H-tetrazol-2-yl]acetate

### Step 1: N-Benzyl-5-bromopyrimidin-2-amine

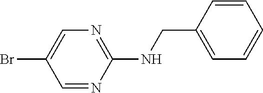

Into a 2 L round-bottom flask equipped with a heating mantle, reflux condenser and under N₂ was added 2-chloro-5-bromopyrimidine (125 g, 646 mmol), DIPEA (251 mL, 1435 mmol) and benzylamine (95 mL, 872 mmol) in 2-propanol (250 mL). The reaction mixture was heated to 100° C. for 1 h and then cooled to room temperature and stirred for 16 h. The crude reaction mixture was filtered under vacuum on a sintered glass funnel, and the filter cake was rinsed with ethanol (2×50 mL) and hexanes (200 mL). The filter cake was further dried under vacuum to provide the title compound as a white crystalline solid.

### Step 2: 2-(Benzylamino)pyrimidine-5-carbonitrile

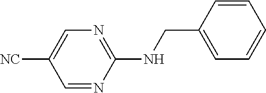

Into a 5 L round-bottom flask equipped with a reflux condenser and heating mantle and under N₂ was added N-benzyl-5-bromopyrimidin-2-amine (150 g, 568 mmol), copper(I) cyanide (64 g, 710 mmol) and DMF (1.5 L). The reaction mixture was heated to 150° C. for 16 h. The reaction mixture was cooled to room temperature and poured into a 3 L separatory funnel containing 750 mL of a 1:1:2 aqueous solution of saturated NH₄Cl:concentrated NH₄OH:water. The aqueous layer was extracted with MeTHF (3×500 mL) and the combined organic layers were washed with brine, dried over Na₂SO₄, filtered and concentrated under reduced pressure. The obtained product was utilized in the subsequent step without further purification.

### Step 1: N-Benzyl-5-(2H-tetrazol-5-yl)pyrimidin-2-amine

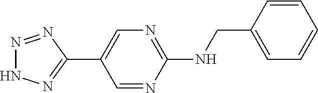

A suspension of 2-(benzylamino)pyrimidine-5-carbonitrile (34 g, 162 mmol), sodium azide (13 g, 202 mmol) and ammonium chloride (35 g, 647 mmol) in DMF (340 mL) was heated at 100° C. A steady flow of N₂ (170 mL/min) was placed above the reaction mixture and the reaction flask was kept open and well-vented. At t=1.5 h, t=3 h and t=4 h, an additional 1 equiv of sodium azide (10.5 g, 162 mmol) was added to the mixture. After 5 h total reaction time, the mixture was allowed to cool to room temperature. The reaction was poured into a 2 L separatory funnel containing aqueous 1 N NaOH solution (750 mL) and the aqueous layer was extracted with MTBE (2×200 mL). The aqueous layer was cooled to 0° C. in an ice bath and acidified to pH 1-2 with aqueous 2 M HCl solution. During the acidification, the internal temperature was maintained below 15° C. The aqueous mixture was poured into a separatory funnel and extracted with EtOAc (3×300 mL). The combined organic layers were washed with brine, dried over Na₂SO₄, filtered and concentrated under reduced pressure to afford to the title compound as a beige solid.

### Step 4: Ethyl {5-[2-(benzylamino)pyrimidin-5-yl]-2H-tetrazol-2-yl}acetate

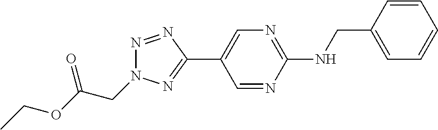

To a 2 L round-bottom flask equipped with a heating mantle and reflux condenser was added N-benzyl-5-(2H-tetrazol-5-yl)pyrimidin-2-amine (31.9 g, 126 mmol), ethyl bromoacetate (21 mL, 188 mmol), triethylamine (35 mL, 251 mmol) and THF (390 mL). The reaction mixture was heated to 65° C. for 1 h and then cooled to room temperature. Water (1 L) was added and the mixture was stirred at room temperature for 1 h, then filtered under vacuum on a sintered glass funnel. The filter cake was further washed with water: THF (2.5:1, 300 mL) and then with water (500 mL). The resulting cake was re-suspended in THF (320 mL) and then water (640 mL) was added gradually over 0.5 h. The suspension was stirred an additional 0.5 h at room temperature and then filtered under vacuum on a sintered glass funnel. The filter cake was washed with 2:1 water:THF (2×200 mL) and dried under vacuum for several hours, affording the title compound as white powder.

### Step 5: Ethyl [5-(2-aminopyrimidin-5-yl)-2H-tetrazol-2-yl]acetate

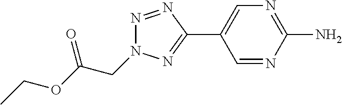

Into a 1 L round-bottom flask was dissolved ethyl {5-[2-(benzylamino)pyrimidin-5-yl]-2H-tetrazol-2-yl}acetate (30.7 g, 90 mmol) in MeCN (300 mL) and water (60 mL). To this solution was added cerium ammonium nitrate (114 g, 208 mmol) portionwise over 15 min. The mixture was stirred at room temperature for 1 h and was poured into a separatory funnel containing water (500 mL). The aqueous layer was extracted with EtOAc (3×250 mL). The combined organic layers were washed with aqueous 0.1 N HCl solution/brine (1:1; 250 mL), brine, dried over Na₂SO₄, filtered and concentrated under reduced pressure to afford the title compound.

### Step 6: Ethyl [5-(2-chloropyrimidin-5-yl)-2H-tetrazol-2-yl]acetate

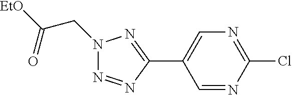

A solution of ethyl [5-(2-aminopyrimidin-5-yl)-2H-tetrazol-2-yl]acetate (16.6 g, 66 mmol) in 1,2-dichloroethane (330 mL) was treated with antimony(III) chloride (19.3 mL, 266 mmol). The mixture was cooled to 0° C. in an ice bath and tert-butyl nitrite (44 mL, 332 mmol) was added dropwise to the reaction mixture over 15 min. After 3 h, the mixture was diluted with saturated aqueous NaHCO₃ solution (200 mL) and CH₂Cl₂ (200 mL) and the resulting suspension was filtered through a pad of celite on a sintered glass funnel under vacuum. The filtrate was poured into a 2 L separatory funnel containing saturated aqueous NaHCO₃ solution (250 mL) and the aqueous layer was extracted with CH₂Cl₂ (3×200 mL). The combined organic layers were washed with brine, dried over Na₂SO₄, filtered and concentrated under reduced pressure. Purification by column chromatography through silica gel, eluting with 85:15 hexanes:EtOAc to 50:50 hexanes:EtOAc as a gradient, afforded the title compound as an off-white solid.

¹H NMR (d₆-DMSO, 400 MHz): δ 9.40 (2H, s), 6.01 (2H, s), 4.24 (2H, q, J=7.0 Hz), 1.25 (3H, t, J=7.0 Hz). MS (ESI, Q⁺) m/z 269, 271 (M+1, ³⁵Cl, ³⁷Cl).

### Intermediate 6

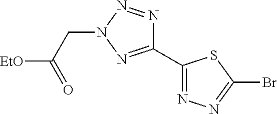

### Ethyl [5-(5-bromo-1,3,4-thiadiazol-2-yl)-2H-tetrazol-2-yl]acetate

### Step 1: 5-Bromo-1,3,4-thiadiazol-2-amine

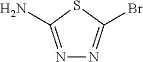

Into a 250 mL round-bottom flask equipped with a magnetic stir bar was added 1,3,4-thiadiazol-2-amine (10.0 g, 99 mmol) and sodium acetate (8.92 g, 109 mmol) in concentrated acetic acid (57 mL). The suspension was treated with dropwise addition of bromine (5.60 mL, 109 mmol) and the yellow-orange suspension was stirred at room temperature for 3 h. The reaction mixture was diluted with water (100 mL) and filtered through filter paper on a Hirsch funnel, washing with water to give a light beige solid.

### Step 2: 5-Bromo-1,3,4-thiadiazole-2-carbonitrile

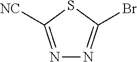

Into a 100 mL round-bottom flask equipped with a magnetic stir bar was added 5-bromo-1,3,4-thiadiazol-2-amine (6.00 g, 33.3 mmol) and copper(I) cyanide (6.57 g, 73.3 mmol) in MeCN (111 mL). The suspension was cooled to 0° C. and tert-butyl nitrite (8.30 mL, 70.0 mmol) was added dropwise over 0.5 h. After stirring at room temperature for an additional 1 h, the reaction mixture was filtered through a pad of silica gel on a sintered glass funnel, washing with ethyl acetate (100 mL). The filtrate was concentrated under reduced pressure and purified by column chromatography on silica gel, eluting with 0% EtOAc in hexanes to 50% EtOAc in hexanes as a gradient. The desired product was obtained as an off-white solid.

### Step 3: 5-(5-Bromo-1,3,4-thiadiazol-2-yl)-1H-tetrazole

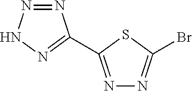

To a suspension of 5-bromo-1,3,4-thiadiazole-2-carbonitrile (1.0 g, 5.0 mmol) and ZnBr₂ (1.1 g, 5.0 mmol) in 2-propanol (10 mL) and H₂O (5 mL) was added NaN₃ (0.65 g, 10 mmol) in a sealed tube. The mixture was stirred at 120° C. for 16 h and then cooled to room temperature. The mixture was adjusted to pH=4 with 2 N aqueous HCl solution and extracted with EtOAc (3×50 mL). The combined organic layers were dried over Na₂SO₄, filtered and concentrated in vacuo to afford the unpurified 5-(5-bromo-1,3,4-thiadiazol-2-yl)-1H-tetrazole.

¹³C NMR (d₆-DMSO, 75 MHz) δ 159.1, 150.7, 142.8.

### Step 4: Ethyl [5-(5-bromo-1,3,4-thiadiazol-2-yl)-2H-tetrazol-2-yl]acetate

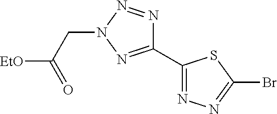

To a solution of 5-(5-bromo-1,3,4-thiadiazol-2-yl)-1H-tetrazole (1.0 g, 4.3 mmol) in DMF (20 mL) was added Cs₂CO₃ (2.1 g, 6.45 mmol) and ethyl bromoacetate (0.95 mL, 8.6 mmol). The resulting solution was stirred at 90° C. for 1 h. The mixture was partitioned between EtOAc (100 mL) and water (200 mL). The aqueous layer was extracted with EtOAc (2×75 mL). The combined organic layers were dried over anhydrous Na₂SO₄, filtered and concentrated in vacuo. Column chromatography on silica gel, eluting with a mixture of EtOAc and hexanes, afforded the title compound as a white solid, together with the 1-alkylated isomer ethyl [5-(5-bromo-1,3,4-thiadiazol-2-yl)-1H-tetrazol-1-yl]acetate.

¹H NMR (CDCl₃, 300 MHz): δ 5.70 (2H, s), 4.26 (2H, q, J=7.0 Hz), 1.28 (3H, t, J=7.0 Hz).

### Intermediate 7

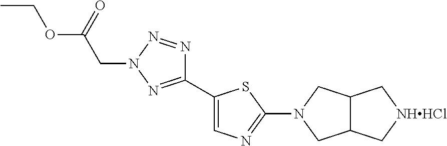

### Ethyl {5-[2-(hexahydropyrrolo[3,4-c]pyrrol-2(1H)-yl)-1,3-thiazol-5-yl]-2H-tetrazol-2-yl}acetate hydrochloride

### Step 1: tert-Butyl 5-{5-[2-(2-ethoxy-2-oxoethyl)-2H-tetrazol-5-yl]-1,3-thiazol-2-yl}hexahydropyrrolo[3,4-c]pyrrole-2(1H)-carboxylate

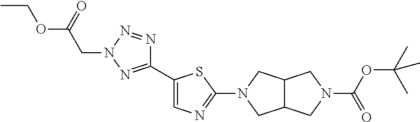

Into a 10 mL round-bottom flask was added ethyl [5-(2-bromo-1,3-thiazol-5-yl)-2H-tetrazol-2-yl]acetate (Intermediate 1, 509 mg, 1.60 mmol), tert-butyl hexahydropyrrolo[3,4-c]pyrrole-2(1H)-carboxylate hydrochloride (453 mg, 1.82 mmol), NMP (3 mL) and DBU (0.55 mL, 3.65 mmol). The resulting mixture was heated at 110° C. using a microwave reactor for 30 min. The reaction was cooled to room temperature and then partitioned between EtOAc and half-saturated aqueous NaHCO₃. The organic layer was dried over Na₂SO₄, filtered and concentrated under reduced pressure. The resulting material was purified by column chromatography through silica gel, eluting with 45% EtOAc in hexanes to 100% EtOAc in hexanes as a gradient. The desired product was obtained as a white solid.

### Step 2: Ethyl {5-[2-(hexahydropyrrolo[3,4-c]pyrrol-2(1H)-yl)-1,3-thiazol-5-yl]-2H-tetrazol-2-yl}acetate hydrochloride

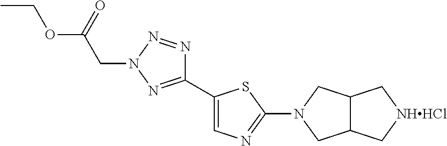

Into a 10 mL round-bottom flask containing tert-butyl 5-{5-[2-(2-ethoxy-2-oxoethyl)-2H-tetrazol-5-yl]-1,3-thiazol-2-yl}hexahydropyrrolo[3,4-c]pyrrole-2(1H)-carboxylate (227 mg, 0.51 mmol) in 1,4-dioxane (1 mL) was added a solution of 4M HCl in 1,4-dioxane (1 mL, 4.0 mmol). The resulting solution was stirred at room temperature for 4 h. The reaction mixture was diluted with Et₂O (10 mL) and vigorously stirred at room temperature for 15 min.

The resulting solid was collected by vacuum filtration and dried under vacuum to give the title compound as a white solid. MS (ESI, Q⁺) m/z 350 (M+1).

The following Examples are provided to illustrate the invention and are not intended to be construed as limiting the scope of the invention in any manner.

### Example 1

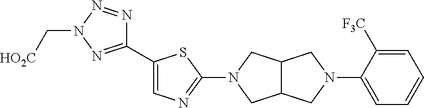

### (5-{2-[5-[2-(Trifluoromethyl)phenyl]hexahydropyrrolo[3,4-c]pyrrol-2(1H)-yl]-1,3-thiazol-5-yl}-2H-tetrazol-2-yl)acetic acid

### Step 1: tert-Butyl 5-[2-(trifluoromethyl)phenyl]hexahydropyrrolo[3,4-c]pyrrole-2(1H)-carboxylate

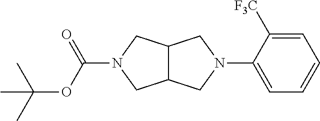

Into a 10 mL flask equipped a magnetic stir bar and a rubber septum was added tert-butyl hexahydropyrrolo[3,4-c]pyrrole-2(1H)-carboxylate hydrochloride (130 mg, 0.52 mmol), palladium(II) acetate (12 mg, 0.05 mmol), racemic-BINAP (65 mg, 0.11 mmol) and sodium tert-butoxide (109 mg, 1.13 mmol). The vial was evacuated under vacuum (10 mm Hg) and backfilled with N₂ (repeated 3 times). Toluene (0.9 mL) and 1-bromo-2-(trifluoromethyl)benzene (145 μL, 0.83 mmol) were added and the solvent was degassed for 10 min with a steady flow of nitrogen before being heated to 115° C. for 16 h. The reaction mixture was partitioned between EtOAc (50 mL) and water (35 mL). The aqueous layer was extracted twice with EtOAc (50 mL). The combined organic layers were dried over Na₂SO₄ and concentrated. The resulting crude material was purified by column chromatography on silica gel, eluting with 5% EtOAc in hexanes to 35% EtOAc in hexanes as a gradient. The desired product was obtained as a white solid.

### Step 2: 2-[2-(Trifluoromethyl)phenyl]octahydropyrrolo[3,4-c]pyrrole hydrochloride

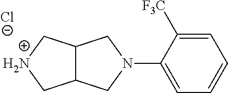

Into a 10 mL round-bottom flask equipped with a magnetic stir bar was added tert-butyl 5-[2-(trifluoromethyl)phenyl]hexahydropyrrolo[3,4-c]pyrrole-2(1H)-carboxylate (131 mg, 0.37 mmol), dioxane (0.7 mL) and 4.0 M HCl in dioxane (0.7 mL, 2.8 mmol). The resulting mixture was stirred at room temperature for 2 h. The suspension was diluted with diethyl ether (3 mL) and sonicated. Solvents were removed in vacuo and the resulting residue was dried under vacuum for 16 h to give a white solid which was used directly in the next step.

### Step 3: tert-Butyl (5-{2-[5-[2-(trifluoromethyl)phenyl]hexahydropyrrolo[3,4-c]pyrrol-2(1H)-yl]-1,3-thiazol-5-yl}-2H-tetrazol-2-yl)acetate

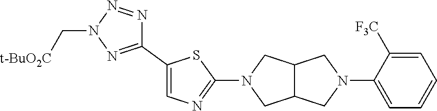

Into a 10 mL flask equipped with a magnetic stir bar and a rubber septum was added tert-butyl [5-(2-bromo-1,3-thiazol-5-yl)-2H-tetrazol-2-yl]acetate (Intermediate 2, 92 mg, 0.27 mmol), 2-[2-(trifluoromethyl)phenyl]octahydropyrrolo[3,4-c]pyrrole hydrochloride (85 mg, 0.29 mmol), NMP (1 mL) and DIPEA (60 μL, 0.34 mmol). The reaction mixture was heated to 120° C. for 0.5 h. The reaction mixture was partitioned between EtOAc (50 mL) and water (35 mL). The aqueous layer was extracted twice with EtOAc (50 mL). The combined organic layers were dried over Na₂SO₄ and concentrated. The resulting crude material was purified by column chromatography on silica gel, eluting with 30% EtOAc in hexanes to 65% EtOAc in hexanes as a gradient. The desired product was obtained as a light yellow solid.

### Step 4: (5-{2-[5-[2-(Trifluoromethyl)phenyl]hexahydropyrrolo[3,4-c]pyrrol-2(1H-yl]-1,3-thiazol-5-yl}-2H-tetrazol-2-yl)acetic acid

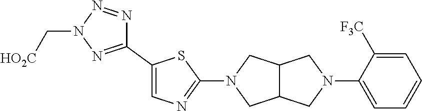

Into a 10 mL round-bottom flask equipped with a magnetic stir bar was added tert-butyl (5-{2-[5-[2-(trifluoromethyl)phenyl]hexahydropyrrolo[3,4-c]pyrrol-2(1H)-yl]-1,3-thiazol-5-yl}-2H-tetrazol-2-yl)acetate (46 mg, 0.09 mmol) and 88% aqueous formic acid (1.5 mL, 39 mmol). The resulting suspension was heated to 100° C. for 1 h, becoming a light yellow solution. The reaction mixture was partitioned between EtOAc (5 mL) and water (3 mL). The aqueous layer was extracted twice with EtOAc (3 mL). The combined organic layers were dried over Na₂SO₄ and concentrated. The resulting residue was dried under vacuum to provide the desired compound as a light yellow foam.

¹H NMR (d₆-acetone, 400 MHz): δ 7.86 (1H, s), 7.65 (1H, d, J=8.0 Hz), 7.58 (1H, t, J=8.0 Hz), 7.45 (1H, d, J=8.5 Hz), 7.19 (1H, t, J=7.5 Hz), 5.62 (2H, s), 3.89 (2H, dd, J=10.5 Hz, 7.0 Hz), 3.53 (2H, dd, J=10.5, 3.5 Hz), 3.45 (2H, dd, J=9.0, 5.5 Hz), 3.31-3.22 (4H, m). MS (ESI, Q⁺) m/z 466 (M+1).

### Example 2

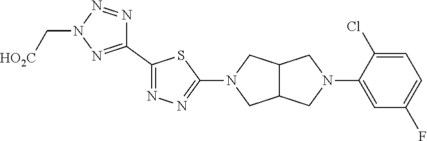

### (5-{5-[5-(2-Chloro-5-fluorophenyl)hexahydropyrrolo[3,4-c]pyrrol-2(1H)-yl]-1,3,4-thiadiazol-2-yl}-2H-tetrazol-2-yl)acetic acid

### Step 1: tert-Butyl 5-(2-chloro-5-fluorophenyl)hexahydropyrrolo[3,4-c]pyrrole-2(1H)-carboxylate

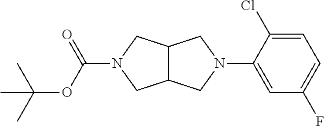

tert-Butyl 5-(2-chloro-5-fluorophenyl)hexahydropyrrolo[3,4-c]pyrrole-2(1H)-carboxylate was prepared following the procedure described in Step 1 of Example 1, but using 2-bromo-1-chloro-4-fluorobenzene to afford the title compound as a white solid.

### Step 2: 2-(2-Chloro-5-fluorophenyl)octahydropyrrolo[3,4-c]pyrrole hydrochloride

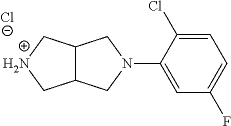

2-(2-Chloro-5-fluorophenyl)octahydropyrrolo[3,4-c]pyrrole hydrochloride was prepared following the procedure described in Step 2 of Example 1, but using tert-butyl 5-(2-chloro-5-fluorophenyl)hexahydropyrrolo[3,4-c]pyrrole-2(1H)-carboxylate to afford the title compound as a white solid.

### Step 3: Ethyl (5-{5-[5-(2-chloro-5-fluorophenyl)hexahydropyrrolo[3,4-c]pyrrol-2(1H)-yl]-1,3,4-thiadiazol-2-yl}-2H-tetrazol-2-yl)acetate

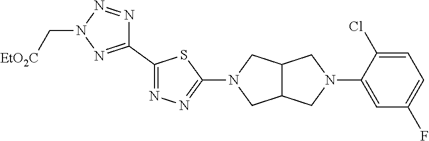

Ethyl (5-{5-[5-(2-chloro-5-fluorophenyl)hexahydropyrrolo[3,4-c]pyrrol-2(1H)-yl]-1,3,4-thiadiazol-2-yl}-2H-tetrazol-2-yl)acetate was prepared following the procedure described in Step 3 of Example 1, but using Intermediate 6 to afford the title compound as a light-yellow solid. MS (ESI, Q⁺) m/z 479 (M+1).

### Step 4: (5-{5-[5-(2-Chloro-5-fluorophenyl)hexahydropyrrolo[3,4-c]pyrrol-2(1H)-yl]-1,3,4-thiadiazol-2-yl}-2H-tetrazol-2-yl)acetic acid

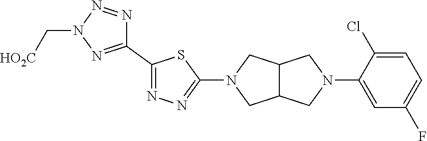

Into a 10 mL round-bottom flask equipped with a magnetic stir bar was added ethyl (5-{5-[5-(2-chloro-5-fluorophenyl)hexahydropyrrolo[3,4-c]pyrrol-2(1H)-yl]-1,3,4-thiadiazol-2-yl}-2H-tetrazol-2-yl)acetate (206 mg, 0.43 mmol), THF (1.5 mL), MeOH (0.7 mL) and 1N aqueous LiOH solution (0.55 mL, 0.55 mmol). The solution was stirred at room temperature for 1 h and partitioned between EtOAc (25 mL) and water (25 mL) containing 1 N aqueous HCl solution (0.8 mL). The aqueous layer was extracted with EtOAc (2×25 mL) and the combined organic layers were dried over MgSO₄, filtered, concentrated and triturated with EtOAc to give the title compound as a white solid.

¹H NMR (d₆-Acetone, 400 MHz): δ 7.34 (1H, dd, J=9.0, 6.0 Hz), 6.83 (1H, dd, J=11.5, 3.0), 6.69 (1H, ddd, J=9.0, 7.5, 3.0), 5.69 (2H, s), 3.96 (2H, dd, J=10.5, 7.0 Hz), 3.65, (2H, dd, J=10.5, 4.0 Hz), 3.60-3.50 (4H, m), 3.39-3.28 (2H, m). MS (ESI, Q⁺) m/z 451 (M+1).

### Example 3

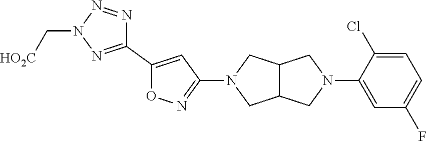

### (5-{3-[5-(2-Chloro-5-fluorophenyl)hexahydropyrrolo[3,4-c]pyrrol-2(1H)-yl]isoxazol-5-yl}-2H-tetrazol-2-yl)acetic acid

### Step 1: 3-[5-(2-Chloro-5-fluorophenyl)hexahydropyrrolo[3,4-c]pyrrol-2(1H)-yl]-4,5-dihydroisoxazole-5-carboxamide

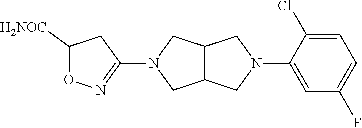

Into a 100 mL round-bottom flask equipped with a magnetic stir bar was added butan-1-ol (22 mL), 3-bromo-4,5-dihydroisoxazole-5-carboxamide (Intermediate 3, 1.16 g, 6.0 mmol), 2-(2-chloro-5-fluorophenyl)octahydropyrrolo[3,4-c]pyrrole hydrochloride (1.10 g, 4.0 mmol) followed by sodium carbonate (1.54 g, 14.5 mmol). The mixture was heated at 110° C. for 16 h. The mixture was cooled and diluted with EtOAc (150 mL). The solid residues were removed by filtration through filter paper under vacuum. The filtrate was concentrated under reduced pressure and the resulting brown residue was used directly in Step 2.

### Step 2: 3-[5-(2-Chloro-5-fluorophenyl)hexahydropyrrolo[3,4-c]pyrrol-2(1H)-yl]isoxazole-5-carboxamide

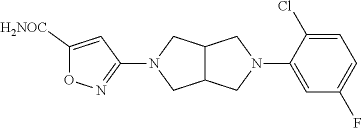

Into a 250 mL round-bottom flask equipped with a reflux condenser and a magnetic stir bar was added the unpurified 3-[5-(2-chloro-5-fluorophenyl)hexahydropyrrolo[3,4-c]pyrrol-2(1H)-yl]-4,5-dihydroisoxazole-5-carboxamide from Step 1 (1.4 g, 4.0 mmol), toluene (65 mL) imidazole (0.81 g, 11.9 mmol) and iodine (1.51 g, 6.0 mmol). The mixture was refluxed for 16 h. The mixture was cooled, poured into a 250 mL separatory funnel containing water (100 mL) and the mixture was extracted with ethyl acetate (3×70 mL). The combined organic layers were washed with brine, dried over MgSO₄, filtered and the solvent was evaporated under reduced pressure. Purification by column chromatography on silica gel, eluting with 20% EtOAc in hexanes to 100% EtOAc in hexanes as a gradient, afforded the title compound as a light brown solid. MS (ESI, Q⁺) m/z of 351 (M+1).

### Step 3: 3-[5-(2-Chloro-5-fluorophenyl)hexahydropyrrolo[3,4-c]pyrrol-2(1H)-yl]isoxazole-5-carbonitrile

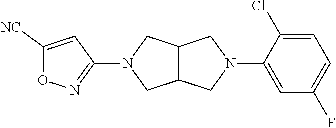

Into a 100 mL flask equipped with a magnetic stir bar was added 3-[5-(2-chloro-5-fluorophenyl)hexahydropyrrolo[3,4-c]pyrrol-2(1H)-yl]-4,5-dihydroisoxazole-5-carboxamide (935 mg, 2.7 mmol), THF (40 mL) and triethylamine (1.4 mL, 10.0 mmol). This solution was cooled in an ice bath and trifluoroacetic anhydride (0.75 mL, 5.3 mmol) was added dropwise. The reaction mixture was allowed to warm to room temperature and stirred for 1 h. A saturated aqueous solution of NaHCO₃ (70 mL) was added and the mixture was extracted with ethyl acetate (3×50 mL). The combined organic layers were washed with brine, dried over MgSO₄, filtered and the solvent was evaporated under reduced pressure. Purification by column chromatography on silica gel, eluting with 5% EtOAc in hexanes to 30% EtOAc in hexanes as a gradient, afforded the title compound as a colorless oil.

### Step 4: 2-(2-Chloro-5-fluorophenyl)-5-[5-(2H-tetrazol-5-yl)isoxazol-3-yl]octahydropyrrolo[3,4-c]pyrrole

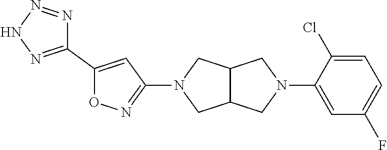

Into a 25 mL pressure flask equipped with a magnetic stir bar was added 3-[5-(2-chloro-5-fluorophenyl)hexahydropyrrolo[3,4-c]pyrrol-2(1H)-yl]isoxazole-5-carbonitrile (715 mg, 2.1 mmol), dioxane (9 mL), DMSO (1 mL), sodium azide (700 mg, 10.8 mmol) and ammonium chloride (577 mg, 10.8 mmol). This suspension was stirred at 110° C. for 16 h. The reaction mixture was allowed to cool to room temperature and it was poured into a 125 mL flask and treated with 1N aqueous HCl solution and stirred for 1 h to become a suspension. The beige solid was collected by vacuum filtration and washed with water. The resulting beige solid was dried under vacuum for 16 h.

### Step 5: tert-Butyl (5-{3-[5-(2-chloro-5-fluorophenyl)hexahydropyrrolo[3,4-c]pyrrol-2(1H)-yl]isoxazol-5-yl}-2H-tetrazol-2-yl)acetate

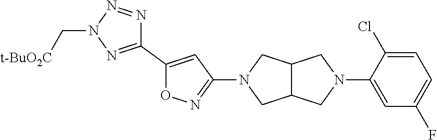

Into a 25 mL flask equipped with a magnetic stir bar was added 2-(2-chloro-5-fluorophenyl)-5-[5-(2H-tetrazol-5-yl)isoxazol-3-yl]octahydropyrrolo[3,4-c]pyrrole (649 mg, 1.7 mmol), THF (9 mL), tert-butyl bromoacetate (0.5 mL, 3.4 mmol) and triethylamine (0.5 mL, 3.6 mmol). The reaction mixture was stirred at 80° C. for 1 h. The cooled suspension was poured into a 250 mL separatory funnel containing water (75 mL) and the mixture was extracted with ethyl acetate (3×50 mL). The combined organic layers were washed with brine, dried over Na₂SO₄, filtered and the solvent was evaporated under reduced pressure. Purification by column chromatography on silica gel, eluting with 25% EtOAc in hexanes to 80% EtOAc in hexanes as a gradient, afforded the title compound as a white solid

### Step 6: (5-{3-[5-(2-Chloro-5-fluorophenyl)hexahydropyrrolo[3,4-c]pyrrol-2(1H)-yl]isoxazol-5-yl}-2H-tetrazol-2-yl)acetic acid

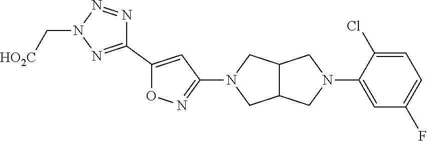

(5-{3-[5-(2-Chloro-5-fluorophenyl)hexahydropyrrolo[3,4-c]pyrrol-2(1H)-yl]isoxazol-5-yl}-2H-tetrazol-2-yl)acetic acid was prepared from tert-butyl (5-{3-[5-(2-chloro-5-fluorophenyl)hexahydropyrrolo[3,4-c]pyrrol-2(1H)-yl]isoxazol-5-yl}-2H-tetrazol-2-yl)acetate following the procedure described in Step 4 of Example 1. Purification by column chromatography on silica gel, eluting with AcOH:EtOH:EtOAc (0:0:100 to 0.8:24:75.2 as a gradient), afforded the title compound as an off-white powder.

¹H NMR (DMSO-d₆, 400 MHz): δ 7.35 (1H, dd, J=9.0, 6.0 Hz), 6.85 (1H, s), 6.84 (1H, dd, J=11.5, 3.0 Hz), 6.70 (1H, ddd, J=9.0, 7.5, 3.0 Hz), 5.70 (2H, s), 3.72 (2H, dd, J=10.0, 7.0 Hz), 3.53 (2H, dd, J=9.5, 7.0 Hz), 3.42-3.37 (4H, m), 3.22-3.16 (2H, m).

MS (ESI, Q⁺) m/z 434 (M+1).

### Example 4

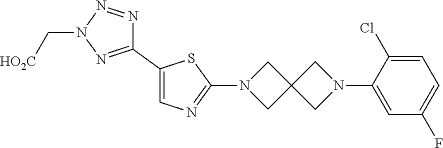

### (5-{2-[6-(2-Chloro-5-fluorophenyl)-2,6-diazaspiro[3.3]hept-2-yl]-1,3-thiazol-5-yl}-2H-tetrazol-2-yl)acetic acid

### Step 1: Oxetane-3,3-diyldimethanamine dihydrobromide

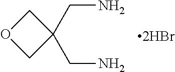

Into a 5 L pressure tank reactor was placed 3,3-bis(bromomethyl)oxetane (350 g, 1.43 mol). To this was added ethanol (3.2 L), followed by addition of ammonia gas. The resulting solution was stirred overnight. The reaction temperature was maintained at 50° C. with a pressure of ammonia gas of 6 atmospheres. The mixture was concentrated under reduced pressure to give the title compound as a beige solid.

¹H NMR (400 MHz, D₂O): δ 4.59 (4H, s), 3.41 (4H, s).

### Step 2: 2,2-Bis(bromomethyl)propane-1,3-diamine dihydrobromide

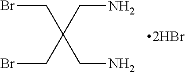

Into a 5 L, 3-neck, round bottom flask was placed oxetane-3,3-diyldimethanamine dihydrobromide (760 g, 2.73 mol) followed by phosphorous tribromide (3 L). The resulting solution was stirred for 5 days while the temperature was maintained at reflux temperature in a 175° C. oil bath. The bulk of the phosphorous tribromide was removed by distillation at 100-120° C. under reduced pressure until the reaction mixture became a thick mass. The remaining phosphorous tribromide was quenched by adding the reaction mixture to crushed ice followed by the addition of MeOH (1.5 L). The reaction mixture was filtered through filter paper and the filter cake was washed once with MeOH (1 L). The title compound was obtained as a light brown solid.

### Step 3: Di-tert-butyl 2,6-diazaspiro[3.3]heptane-2,6-dicarboxylate

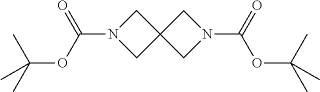

Into a 20 L, 4-neck, round bottom flask was placed 2,2-bis(bromomethyl)propane-1,3-diamine dihydrobromide (400 g, 947 mmol), H₂O (7.2 L) followed by the addition of a solution of NaOH (114 g, 2.85 mol) in H₂O (800 mL) dropwise with stirring, while warming to a temperature of 95-100° C. The resulting solution was stirred for 4 h while the temperature was maintained at 95-100° C. Na₂CO₃ (251 g, 2.37 mol) was then added while cooling the reaction mixture to room temperature. The reaction mixture was diluted with THF (3.6 L), then di-tert-butyl dicarbonate (414 g, 1.90 mol) was added and the resulting solution was stirred for 16 h at room temperature. The reaction mixture was then extracted with EtOAc (2×2 L). The combined organic layers were dried over Na₂SO₄ and concentrated under reduced pressure. The resulting residue was triturated with petroleum ether and filtered to give the title compound as a white solid.

¹H NMR (300 MHz, CDCl₃): δ 4.05 (8H, s), 1.46 (18H, s).

### Step 4: tert-Butyl 2,6-diazaspiro[3.3]heptane-2-carboxylate oxalate

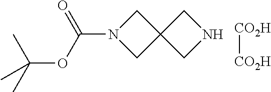

Into a 5 L, 3-neck, round bottom flask, purged and maintained with an inert atmosphere of nitrogen, was placed a solution of acetyl chloride (25.3 g, 322.3 mmol) in methanol (2.7 L). To this was added di-tert-butyl 2,6-diazaspiro[3.3]heptane-2,6-dicarboxylate (96 g, 321 mmol). The resulting solution was stirred for 20 h while the temperature was maintained at 20° C. Solid KOH (18.1 g, 323.2 mmol) was added in small portions in order to maintain the temperature below 30° C. The resulting mixture was concentrated under reduced pressure. The residue was transferred to a separatory funnel using water (200 mL) and EtOAc (1 L). The aqueous layer was extracted with EtOAc (2×500 mL). The combined organic layers were dried over Na₂SO₄ and concentrated under reduced pressure. To the residue was added 1 L of ether and oxalic acid (28.9 g, 321 mmol). The resulting solution was stirred for 1 h at room temperature. The resulting solid was collected by vacuum filtration, washed once with EtOAc (500 mL) and once with ether (1 L) to give the title compound as a white solid.

¹H NMR (400 MHz, D₂O) δ 4.20 (4H, s), 4.07 (4H, s), 1.31 (9H, s).

MS (ESI, Q⁺) m/z 199 (M+1).

### Step 5: tert-Butyl 6-(2-chloro-5-fluorophenyl)-2,6-diazaspiro[3.3]heptane-2-carboxylate

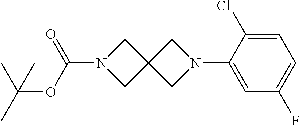

Into a sealed tube, a mixture of tert-butyl 2,6-diazaspiro[3.3]heptane-2-carboxylate oxalate (250 mg, 0.87 mmol), 1-chloro-4-fluoro-2-iodobenzene (347 mg, 1.35 mmol), palladium(II) acetate (19 mg, 0.085 mmol), racemic-BINAP (108 mg, 0.17 mmol) and sodium tert-butoxide (275 mg, 2.86 mmol) in toluene (1.8 mL) was degassed by bubbling N₂ for 5 min. The vial was sealed with a cap and the mixture was heated to 120° C. for 16 h. The reaction was diluted with EtOAc, poured into saturated aqueous NaHCO₃, extracted with EtOAc, and the organic layer was washed with saturated aqueous NaHCO₃ and brine, dried over Na₂SO₄, filtered and concentrated. The resulting crude material was purified twice by column chromatography through silica gel, eluting with 0% EtOAc in hexanes to 40% EtOAc in hexanes as a gradient. The title product was obtained as a colorless oil.

MS (ESI, Q⁺) m/z 327 (M+1).

### Step 6: 2-(2-Chloro-5-fluorophenyl)-2,6-diazaspiro[3.3]heptane trifluoroacetate

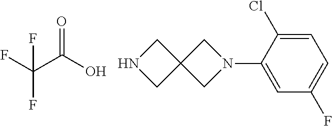

Into a 25 mL round-bottom flask equipped with a magnetic stir bar, a solution of tert-butyl 6-(2-chloro-5-fluorophenyl)-2,6-diazaspiro[3.3]heptane-2-carboxylate (208 mg, 0.64 mmol) in CH₂Cl₂ (4 mL) was treated with TFA (2 mL, 26 mmol). The solution was stirred for 1 h at room temperature. Solvents were removed in vacuo and the resulting residue was triturated with a mixture of Et₂O/heptane to give the title compound as an off-white solid.

MS (ESI, Q⁺) m/z 227 (M+1).

### Step 7: tert-Butyl (5-{2-[6-(2-chloro-fluorophenyl)-2,6-diazaspiro[3.3]hept-2-yl]-1,3-thiazol-5-yl}-2H-tetrazol-2-yl)acetate

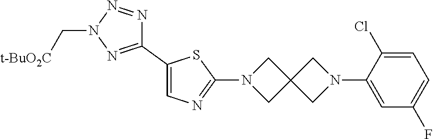

Into a sealed tube, a solution of tert-butyl [5-(2-bromo-1,3-thiazol-5-yl)-2H-tetrazol-2-yl]acetate (Intermediate 2, 80 mg, 0.23 mmol) and 2-(2-chloro-5-fluorophenyl)-2,6-diazaspiro[3.3]heptane trifluoroacetate (102 mg, 0.30 mmol) in NMP (1.25 mL) was treated with DBU (87 μL, 0.58 mmol). The tube was sealed and immersed into a preheated oil bath at 130° C., and stirred at this temperature for 20 min. The reaction was diluted with EtOAc, poured into aqueous 0.5 N HCl solution, extracted with EtOAc, and the organic layer was washed with water and brine, dried over Na₂SO₄, filtered and concentrated. The resulting material was purified by column chromatography on silica gel, eluting with 0% EtOAc in hexanes to 60% EtOAc in hexanes as a gradient. The title product was obtained as a waxy oil.

MS (ESI, Q⁺) m/z 492 (M+1).

### Step 8: (5-{2-[6-(2-Chloro-5-fluorophenyl)-2,6-diazaspiro[3.3]hept-2-yl]-1,3-thiazol-5-yl}-2H-tetrazol-2-yl)acetic acid

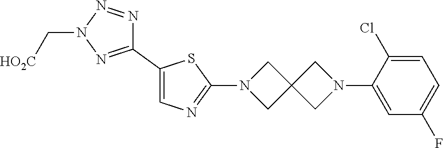

Into a 10 mL round-bottom flask equipped with a magnetic stir bar, a solution of tert-butyl (5-{2-[6-(2-chloro-5-fluorophenyl)-2,6-diazaspiro[3.3]hept-2-yl]-1,3-thiazol-5-yl}-2H-tetrazol-2-yl)acetate (56 mg, 0.11 mmol) in CH₂Cl₂ (2 mL) was treated with TFA (1 mL, 13 mmol). The final solution was stirred for 4 h at room temperature. Solvents were removed in vacuo and the resulting residue was triturated with a mixture of Et₂O/heptane to give the title compound as a white solid.

¹H NMR (400 MHz, DMSO-d₆): δ 13.78 (1H, br s), 7.87 (1H, s), 7.27 (1H, dd, J=9.0, 6.0 Hz), 6.60 (1H, td, J=8.5, 3.0 Hz), 6.46 (1H, dd, J=11.0, 3.0 Hz), 5.71 (2H, s), 4.33 (4H, s), 4.27 (4H, s). MS (ESI, Q⁺) m/z 436 (M+1).

### Example 5

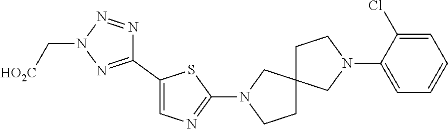

### (5-{2-[7-(2-Chlorophenyl)-2,7-diazaspiro[4.4]non-2-yl]-1,3-thiazol-5-yl}-2H-tetrazol-2-yl)acetic acid

### Step 1: tert-Butyl (5-{2-[7-(2-chlorophenyl)-2,7-diazaspiro[4.4]non-2-yl]-1,3-thiazol-5-yl}-2H-tetrazol-2-yl)acetate

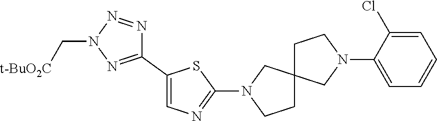

tert-Butyl (5-{2-[7-(2-chlorophenyl)-2,7-diazaspiro[4.4]non-2-yl]-1,3-thiazol-5-yl}-2H-tetrazol-2-yl)acetate was prepared following the procedure described in Step 7 of Example 4, but using 2-(2-chlorophenyl)-2,7-diazaspiro[4.4]nonane to afford the title compound as a waxy oil. MS (ESI, Q⁺) m/z 502 (M+1).

### Step 2: (5-{2-[7-(2-Chlorophenyl)-2,7-diazaspiro[4.4]non-2-yl]-1,3-thiazol-5-yl}-2H-tetrazol-2-yl)acetic acid

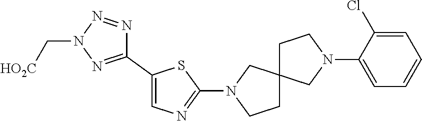

Into a 25 mL round-bottom flask equipped with a magnetic stir bar, water (0.4 mL) and formic acid (1.6 mL, 42 mmol) were added to tert-butyl (5-{2-[7-(2-chlorophenyl)-2,7-diazaspiro[4.4]non-2-yl]-1,3-thiazol-5-yl}-2H-tetrazol-2-yl)acetate (72 mg, 0.14 mmol) and the final solution was immersed into a preheated oil bath at 100° C. for 1 h. The reaction was diluted with EtOAc, poured into water, extracted with EtOAc, and the organic layer was washed with water and brine, dried over Na₂SO₄, filtered and concentrated. The resulting material was dissolved in EtOAc and filtered through a pad of Celite. Solvent was removed in vacuo and the resulting residue was triturated with a mixture of Et₂O/heptane to give the title compound as a white solid.

¹H NMR (500 MHz, DMSO-d₆): δ 13.75 (1H, br s), 7.87 (1H, s), 7.31 (1H, d, J=7.5 Hz), 7.22-7.17 (1H, m), 6.99 (1H, d, J=8.0 Hz), 6.87-6.79 (1H, m), 5.68 (2H, s), 3.64-3.54 (3H, m), 3.54-3.43 (3H, m), 3.43-3.30 (2H, m), 2.18-2.07 (2H, m), 2.06-1.95 (2H, m).

MS (ESI, Q⁺) m/z 446 (M+1).

### Example 6

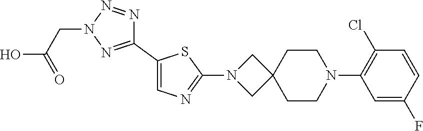

### (5-{2-[7-(2-Chloro-5-fluorophenyl)-2,7-diazaspiro[3.5]non-2-yl]-1,3-thiazol-5-yl}-2H-tetrazol-2-yl)acetic acid

### Step 1: tert-Butyl 7-(2-chloro-5-fluorophenyl)-2,7-diazaspiro[3.5]nonane-2-carboxylate

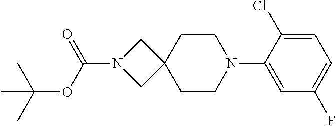

tert-Butyl 7-(2-chloro-5-fluorophenyl)-2,7-diazaspiro[3.5]nonane-2-carboxylate was prepared following the procedure described in Step 5 of Example 4, but using 2-(tert-butylbutoxycarbonyl)-2,7-diazaspiro[3.5]nonane to afford the title compound as a yellow oil.

MS (ESI, Q⁺) m/z 355 (M+1).

### Step 2: 7-(2-Chloro-5-fluorophenyl)-2,7-diazaspiro[3.5]nonane trifluoroacetate

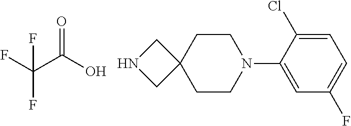

7-(2-Chloro-5-fluorophenyl)-2,7-diazaspiro[3.5]nonane trifluoroacetate was prepared following the procedure described in Step 6 of Example 4, but using tert-butyl 7-(2-chloro-5-fluorophenyl)-2,7-diazaspiro[3.5]nonane-2-carboxylate to afford the title compound as an off-white solid. MS (ESI, Q⁺) m/z 255 (M+1).

### Step 3: tert-Butyl (5-{2-[7-(2-chloro-5-fluorophenyl)-2,7-diazaspiro[3.5]non-2-yl]-1,3-thiazol-5-yl}-2H-tetrazol-2-yl)acetate

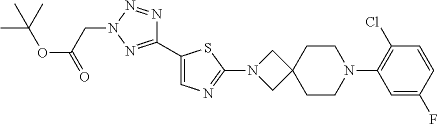

tert-Butyl (5-{2-[7-(2-chloro-5-fluorophenyl)-2,7-diazaspiro[3.5]non-2-yl]-1,3-thiazol-5-yl}-2H-tetrazol-2-yl)acetate was prepared following the procedure described in Step 7 of Example 4, but using 7-(2-chloro-5-fluorophenyl)-2,7-diazaspiro[3.5]nonane trifluoroacetate to afford the title compound as a white solid.

MS (ESI, Q⁺) m/z 520 (M+1).

### Step 4: (5-{2-[7-(2-Chloro-5-fluorophenyl)-2,7-diazaspiro[3.5]non-2-yl]-1,3-thiazol-5-yl}-2H-tetrazol-2-yl)acetic acid

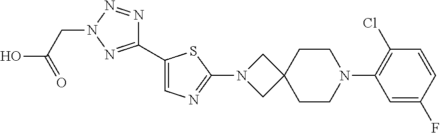

(5-{2-[7-(2-Chloro-5-fluorophenyl)-2,7-diazaspiro[3.5]non-2-yl]-1,3-thiazol-5-yl}-2H-tetrazol-2-yl)acetic acid was prepared following the procedure described in Step 8 of Example 4, but using tert-butyl (5-{2-[7-(2-chloro-5-fluorophenyl)-2,7-diazaspiro[3.5]non-2-yl]-1,3-thiazol-5-yl}-2H-tetrazol-2-yl)acetate to afford the title compound as an off-white solid. ¹H NMR (500 MHz, DMSO-d₆): δ 13.78 (1H, br s), 7.86 (1H, s), 7.45 (1H, dd, J=9.0, 6.0 Hz), 7.00 (1H, dd, J=10.5, 3.0 Hz), 6.90 (1H, td, J=8.5, 3.0 Hz), 5.70 (2H, s), 3.91 (4H, s), 2.96 (4H, br s), 1.97 (4H, t, J=5.0 Hz). MS (ESI, Q⁺) m/z 464 (M+1).

### Example 7

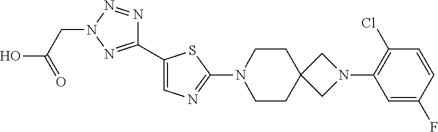

### (5-{2-[2-(2-Chloro-5-fluorophenyl)-2,7-diazaspiro[3.5]non-7-yl]-1,3-thiazol-5-yl}-2H-tetrazol-2-yl)acetic acid

### Step 1: tert-Butyl 2-(2-chloro-5-fluorophenyl)-2,7-diazaspiro[3.5]nonane-7-carboxylate

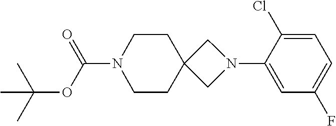

tert-Butyl 2-(2-chloro-5-fluorophenyl)-2,7-diazaspiro[3.5]nonane-7-carboxylate was prepared following the procedure described in Step 5 of Example 4, but using 7-(tert-butoxycarbonyl)-2,7-aza-2-azoniaspirodiazaspiro[3.5]nonane to afford the title compound as a white solid. MS (ESI, Q⁺) m/z 299 (M−tBu).

### Step 2: 2-(2-Chloro-5-fluorophenyl)-2,7-diazaspiro[3.5]nonane trifluoroacetate

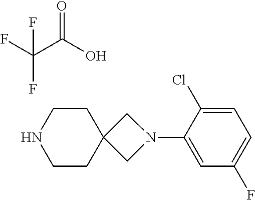

2-(2-Chloro-5-fluorophenyl)-2,7-diazaspiro[3.5]nonane trifluoroacetate was prepared following the procedure described in Step 6 of Example 4, but using tert-butyl 2-(2-chloro-5-fluorophenyl)-2,7-diazaspiro[3.5]nonane-7-carboxylate to afford the title compound as an off-white solid. MS (ESI, Q⁺) m/z 255 (M+1).

### Step 3: tert-Butyl (5-{2-[2-(2-chloro-5-fluorophenyl)-2,7-diazaspiro[3.5]non-7-yl]-1,3-thiazol-5-yl}-2H-tetrazol-2-yl)acetate

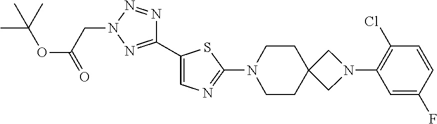

tert-Butyl (5-{2-[2-(2-chloro-5-fluorophenyl)-2,7-diazaspiro[3.5]non-7-yl]-1,3-thiazol-5-yl}-2H-tetrazol-2-yl)acetate was prepared following the procedure described in Step 7 of Example 4, but using 2-(2-chloro-5-fluorophenyl)-2,7-diazaspiro[3.5]nonane trifluoroacetate to afford the title compound as a foamy solid.

MS (ESI, Q⁺) m/z 520 (M+1).

### Step 4: (5-{2-[2-(2-Chloro-5-fluorophenyl)-2,7-diazaspiro[3.5]non-7-yl]-1,3-thiazol-5-yl}-2H-tetrazol-2-yl)acetic acid

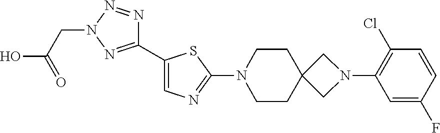

(5-{2-[2-(2-Chloro-5-fluorophenyl)-2,7-diazaspiro[3.5]non-7-yl]-1,3-thiazol-5-yl}-2H-tetrazol-2-yl)acetic acid was prepared following the procedure described in Step 8 of Example 4, but using tert-butyl (5-{2-[2-(2-chloro-5-fluorophenyl)-2,7-diazaspiro[3.5]non-7-yl]-1,3-thiazol-5-yl}-2H-tetrazol-2-yl)acetate to afford the title compound as an off-white solid.

¹H NMR (500 MHz, DMSO-d₆): δ 13.77 (1H, br s), 7.87 (1H, s), 7.25 (1H, dd, J=8.5, 6.0 Hz), 6.56 (1H, td, J=8.5, 3.0 Hz), 6.41 (1H, dd, J=11.0, 3.0 Hz), 5.70 (2H, s), 3.87 (4H, s,), 3.56 (4H, t, J=5.5 Hz), 1.90 (4H, t, J=5.5 Hz). MS (ESI, Q⁺) m/z 464 (M+1).

### Example 8

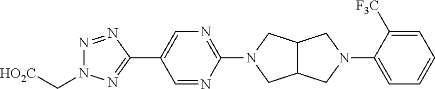

### (5-{2-[5-[2(Trifluoromethyl)phenyl]hexahydropyrrolo[3,4-c]pyrrol-2(1H)-yl]pyrimidin-5-yl}-2H-tetrazol-2-yl)acetic acid

Ethyl [5-(2-chloropyrimidin-5-yl)-2H-tetrazol-2-yl]acetate (Intermediate 5, 750 mg, 2.79 mmol) was added to a 125 mL Erlenmeyer flask and dissolved in 25 mL of dioxane, creating a 0.112 M stock solution. To a 5 mL screw top test tube was added 2-[2-(trifluoromethyl)phenyl]octahydropyrrolo[3,4-c]pyrrole hydrochloride from Example 1, Step 2 (43 mg, 0.15 mmol), along with a magnetic stir bar. 1 mL of the 0.112 M stock solution was added to the test tube, followed by potassium carbonate (37 mg, 0.27 mmol). A cap was fixed tightly to the test tube, and the tube was heated on a magnetic stir plate at 70° C. for 18 h. The cooled test tube was treated with 0.56 mL of methanol and 0.56 mL of a 1N aqueous LiOH solution. The reaction was stirred at room temperature for 16 h. The stir bar was removed and the solvent was removed using a centrifugal evaporator. The residue was dissolved in 1.2 mL of DMSO and purified using mass-directed LC/MS, using a gradient of 40:60 (acetonitrile: 0.5% aqueous ammonium acetate), to 80:20 (acetonitrile: 0.5% aqueous ammonium acetate), and a Synergi Max-RP Axia™ 50×21.2 mm 4 micron preparative HPLC column.

¹H NMR (d₆-DMSO, 500 MHz): δ 8.96 (2H, s), 8.18 (1H, s), 7.64-7.53 (2H, m), 7.37 (1H, d, J=8.0 Hz), 7.14 (1H, d, J=8.0 Hz), 5.20 (2H, s), 3.99-3.93 (2H, m), 3.59 (2H, d, J=12.0 Hz), 3.23-3.10 (4H, m), 3.08-3.03 (2H, m). MS (ESI, Q⁺) m/z 461 (M+1).

### Example 9

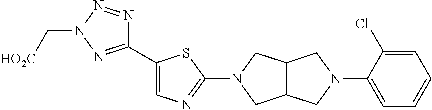

### (5-{2-[5-(2-Chlorophenyl)hexahydropyrrolo[3,4-c]pyrrol-2(1H)-yl]-1,3-thiazol-5-yl}-2H-tetrazol-2-yl)acetic acid

### Step 1: Ethyl (5-{2-[5-(2-chlorophenyl)hexahydropyrrolo[3,4-c]pyrrol-2(1H)-yl]-1,3-thiazol-5-yl}-2H-tetrazol-2-yl)acetate

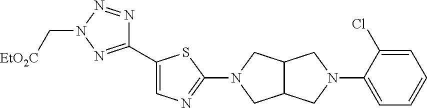

Into a 10 mL flask equipped a magnetic stir bar and a rubber septum was added ethyl {5-[2-(hexahydropyrrolo[3,4-c]pyrrol-2(1H)-yl)-1,3-thiazol-5-yl]-2H-tetrazol-2-yl}acetate hydrochloride (Intermediate 7, 64 mg, 0.17 mmol), palladium(II) acetate (3.8 mg, 0.017 mmol), racemic-BINAP (20.7 mg, 0.033 mmol) and cesium carbonate (115 mg, 0.35 mmol). The flask was evacuated under vacuum (10 mm Hg) and backfilled with N₂ (repeated 3 times). A solution of 1-chloro-2-iodobenzene (35 μL, 0.29 mmol) in toluene (1 mL) was added to the reaction flask and the mixture was degassed for 10 min with a steady flow of nitrogen before being heated to 115° C. for 16 h. The resulting beige heterogeneous mixture was partitioned between EtOAc (50 mL) and water (35 mL). The aqueous layer was extracted twice with EtOAc (50 mL). The combined organic layers were dried over Na₂SO₄ and concentrated. The resulting crude material was purified by column chromatography on silica gel, eluting with 20% EtOAc in hexanes to 70% EtOAc in hexanes as a gradient. The desired product was obtained as a light-yellow gum.

### Step 2: (5-{2-[5-(2-Chlorophenyl)hexahydropyrrolo[3,4-c]pyrrol-2(1H)-yl]-1,3-thiazol-5-yl}-2H-tetrazol-2-yl)acetic acid

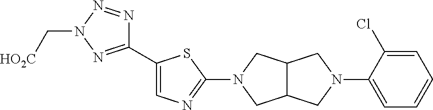

(5-{2-[5-(2-Chlorophenyl)hexahydropyrrolo[3,4-c]pyrrol-2(1H)-yl]-1,3-thiazol-5-yl}-2H-tetrazol-2-yl)acetic acid was prepared following the procedure described in Step 4 of Example 2 to give the title compound as a white solid.

¹H NMR (Acetone-d₆, 400 MHz): δ 7.85 (1H, s), 7.34 (1H, dd, J=8.0, 1.5 Hz), 7.24 (1H, ddd, J=8.0, 7.5, 1.5 Hz), 7.09 (1H, dd, J=8.0, 1.5 Hz), 6.93 (1H, ddd, J=8.0, 7.5, 1.5 Hz), 5.60 (2H, s), 3.92-3.86 (2H, m), 3.57 (2H, dd, J=10.5, 4.0 Hz), 3.45 (4H, d, J=4.5 Hz), 3.31-3.25 (2H, m), MS (ESI, Q⁺) m/z 432 (M+1).

### Example 10

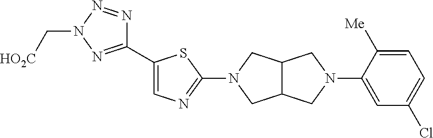

### (5-{2-[5-(5-Chloro-2-methylphenyl)hexahydropyrrolo[3,4-c]pyrrol-2(1H)-yl]-1,3-thiazol-5-yl}-2H-tetrazol-2-yl)acetic acid

### Step 1: tert-Butyl 5-(5-chloro-2-methylphenyl)hexahydropyrrolo[3,4-c]pyrrole-2(1H)-carboxylate

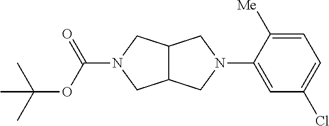

Into a 50 mL flask equipped a magnetic stir bar and a rubber septum was added tert-butyl hexahydropyrrolo[3,4-c]pyrrole-2(1H)-carboxylate oxalate (2.00 g, 6.62 mmol), palladium(II) acetate (149 mg, 0.66 mmol), racemic-BINAP (824 mg, 1.32 mmol) and sodium tert-butoxide (1.91 g, 19.9 mmol). The vial was evacuated under vacuum (10 mm Hg) and backfilled with N₂ (repeated 3 times). Toluene (14 mL) and 2-bromo-4-chlorotoluene (2.18 g, 10.6 mmol) were added to the flask and the solvent was degassed for 10 min with a steady flow of nitrogen before being heated to 115° C. for 20 h. The reaction mixture was partitioned between EtOAc (200 mL) and water (150 mL). The aqueous layer was extracted with EtOAc (5×50 mL) and the combined organic layers were washed with brine, dried over Na₂SO₄ and concentrated. The resulting pale brown oil was purified by column chromatography on silica gel, eluting with 0% EtOAc in hexanes to 40% EtOAc in hexanes as a gradient. The desired product was obtained as a yellow oil.

### Step 2: 2-(5-Chloro-2-methylphenyl)octahydropyrrolo[3,4-c]pyrrole hydrochloride

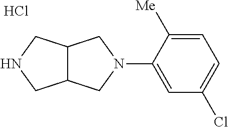

Into a 50 mL round-bottom flask equipped with a magnetic stir bar was added tert-butyl 5-(5-chloro-2-methylphenyl)hexahydropyrrolo[3,4-c]pyrrole-2(1H)-carboxylate (1.91 g, 5.67 mmol), dioxane (6.3 mL) and 4.0 M HCl in 1,4-dioxane (14.0 mL, 56.0 mmol). The resulting mixture was stirred at room temperature for 2 h, then evaporated to dryness in vacuo. The remaining solid was triturated with EtOAc and toluene. Solvents were removed in vacuo and the resulting residue was dried under vacuum for 16 h to give a white solid which was used directly in the next step.

### Step 3: tert-Butyl (5-{2-[5-(5-chloro-2-methylphenyl)hexahydropyrrolo[3,4-c]pyrrol-2(1H)-yl]-1,3-thiazol-5-yl}-2H-tetrazol-2-yl)acetate

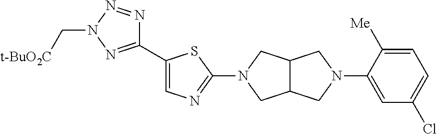

Into a 10 mL flask equipped with a magnetic stir bar and a rubber septum was added tert-butyl [5-(2-bromo-1,3-thiazol-5-yl)-2H-tetrazol-2-yl]acetate (Intermediate 2, 128 mg, 0.37 mmol), 2-(5-chloro-2-methylphenyl)octahydropyrrolo[3,4-c]pyrrole hydrochloride (182 mg, 0.67 mmol), NMP (0.6 mL) and DIPEA (0.25 mL, 1.43 mmol). The reaction mixture was heated to 110° C. for 17 h. The reaction mixture applied directly on a chromatography column packed with silica gel and eluted with 20% EtOAc in hexanes to 85% EtOAc in hexanes as a gradient. The desired product was obtained as a light yellow solid.

MS (ESI, Q⁺) m/z 502 (M+1).

### Step 4: (5-{2-[5-(5-Chloro-2-methylphenyl)hexahydropyrrolo[3,4-c]pyrrol-2(1H)-yl]-1,3-thiazol-5-yl}-2H-tetrazol-2-yl)acetic acid

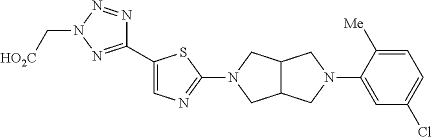

Into a 10 mL round-bottom flask equipped with a magnetic stir bar was added tert-butyl (5-{2-[5-(5-chloro-2-methylphenyl)hexahydropyrrolo[3,4-c]pyrrol-2(1H)-yl]-1,3-thiazol-5-yl}-2H-tetrazol-2-yl)acetate (90 mg, 0.18 mmol), THF (2.0 mL), MeOH (0.5 mL) and 1N aqueous LiOH (0.3 mL, 0.3 mmol). The resulting suspension was stirred at room temperature for 16 h. The reaction mixture was partitioned between EtOAc (30 mL), water (15 mL) and 1N aqueous HCl solution (0.4 mL). The aqueous layer was extracted with EtOAc (2×5 mL). The combined organic layers were dried over Na₂SO₄ and concentrated. The resulting yellow residue was suspended in EtOAc (5 mL), triturated, the solid collected by vacuum filtration, which was then dried under vacuum to give the title compound as a white solid.

¹H NMR (d₆-DMSO+d₆-Acetone (1:9), 400 MHz): δ 7.85 (1H, s), 7.12 (1H, d, J=8.0 Hz), 6.95 (1H, d, J=2.0 Hz), 6.88 (1H, dd, J=8.0, 2.0 Hz), 5.54 (2H, s), 3.88 (2H, dd, J=10.5, 6.3 Hz), 3.56 (2H, dd, J=10.5, 3.0 Hz), 3.38-3.23 (6H, m), 2.27 (3H, s).

MS (ESI, Q⁺) m/z 446 (M+1).

### Example 11

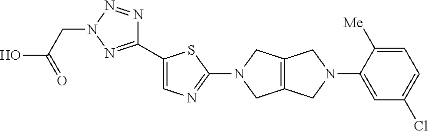

### (5-{2-[5-(5-Chloro-2-methylphenyl)-3,4,5,6-tetrahydropyrrolo[3,4-c]pyrrol-2(1H)-yl]-1,3-thiazol-5-yl}-2H-tetrazol-2-yl)acetic acid

### Step 1: 1,2,3,4,5,6-Hexahydropyrrolo[3,4-c]pyrrole dihydrobromide

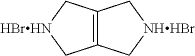

1,2,3,4,5,6-Hexahydropyrrolo[3,4-c]pyrrole dihydrobromide was prepared as described in Jendralla, H.; Fischer, G., Heterocycles 1995, 41, 1291-1298.

### Step 2: tert-Butyl 3,4,5,6-tetrahydropyrrolo[3,4-c]pyrrole-2(1H)-carboxylate ethanedioate

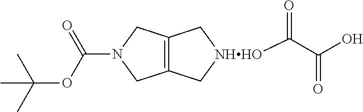

Into a 250-mL 3-necked round-bottom flask was placed a solution of 1,2,3,4,5,6-hexahydropyrrolo[3,4-c]pyrrole dihydrobromide (5.0 g, 16.61 mmol) in CH₃OH/H₂O (50 mL) and NaOH (720 mg, 18.00 mmol). The mixture was stirred for 2 h, then a solution of (Boc)₂O (4.0 g, 18.35 mmol) in CH₃OH (10 mL) was added dropwise with stirring. The resulting solution was stirred for 12 h at room temperature. The pH value of the solution was adjusted to 9 with Na₂CO₃ solution (2 mol/L). The resulting mixture was concentrated under vacuum to remove MeOH. The residual solution was extracted with EtOAc (6×300 mL). The organic layers were combined, dried and concentrated under vacuum. This afforded tert-butyl 3,4,5,6-tetrahydropyrrolo[3,4-c]pyrrole-2(1H)-carboxylate as a yellow oil.

Into a 500-mL round-bottom flask was placed a solution of tert-butyl 3,4,5,6-tetrahydropyrrolo[3,4-c]pyrrole-2(1H)-carboxylate (38.5 g, 165.00 mmol) in EtOAc (200 mL). To the mixture was added oxalic acid (12.8 g, 142.22 mmol). The resulting solution was stirred for 2 hrs at room temperature. The solid was collected by filtration, washed with EtOAc (2×100 mL) and dried. This afforded tert-butyl 3,4,5,6-tetrahydropyrrolo[3,4-c]pyrrole-2(1H)-carboxylate ethanedioate as a pale red solid.

¹H NMR (400 MHz, CD₃OD): δ 4.16 (4H, s), 4.11 (4H, s), 1.51 (9H, s). MS (ESI, Q) m/z 211 (M+1).

### Step 3: tert-Butyl 5-(5-chloro-2-methylphenyl)-3,4,5,6-tetrahydropyrrolo[3,4-c]pyrrole-2(1H)-carboxylate

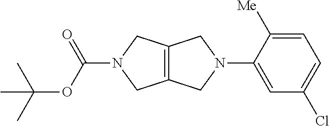

tert-Butyl 5-(5-chloro-2-methylphenyl)-3,4,5,6-tetrahydropyrrolo[3,4-c]pyrrole-2(1H)-carboxylate was prepared following the procedure described in Step 1 of Example 10, but using tert-butyl 3,4,5,6-tetrahydropyrrolo[3,4-c]pyrrole-2(1H)-carboxylate ethanedioate to afford the title compound as a white solid.

### Step 4: 2-(5-Chloro-2-methylphenyl)-1,2,3,4,5,6-hexahydropyrrolo[3,4-c]pyrrole hydrochloride

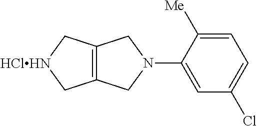

2-(5-Chloro-2-methylphenyl)-1,2,3,4,5,6-hexahydropyrrolo[3,4-c]pyrrole hydrochloride was prepared following the procedure described in Step 2 of Example 10, but using tert-butyl 5-(5-chloro-2-methylphenyl)-3,4,5,6-tetrahydropyrrolo[3,4-c]pyrrole-2(1H)-carboxylate

### Step 5: Ethyl (5-{2-[5-(5-chloro-2-methylphenyl)-3,4,5,6-tetrahydropyrrolo[3,4-c]pyrrol-2(1H)-yl]-1,3-thiazol-5-yl}-2H-tetrazol-2-yl)acetate

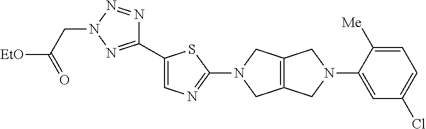

The title compound was prepared following the procedure described in Step 3 of Example 10, but using 2-(5-chloro-2-methylphenyl)-1,2,3,4,5,6-hexahydropyrrolo[3,4-c]pyrrole hydrochloride and ethyl [5-(2-bromo-1,3-thiazol-5-yl)-2H-tetrazol-2-yl]acetate (Intermediate 1) Ethyl (5-{2-[5-(5-chloro-2-methylphenyl)-3,4,5,6-tetrahydropyrrolo[3,4-c]pyrrol-2(1H)-yl]-1,3-thiazol-5-yl}-2H-tetrazol-2-yl)acetate was obtained as a solid. MS (ESI, Q⁺) m/z 472 (M+1).

### Step 6: (5-{2-[5-(5-Chloro-2-methylphenyl)-3,4,5,6-tetrahydropyrrolo[3,4-c]pyrrol-2(1H)-yl]-1,3-thiazol-5-yl}-2H-tetrazol-2-yl)acetic acid

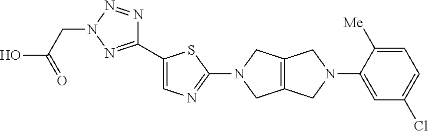

(5-{2-[5-(5-Chloro-2-methylphenyl)-3,4,5,6-tetrahydropyrrolo[3,4-c]pyrrol-2(1H)-yl]-1,3-thiazol-5-yl}-2H-tetrazol-2-yl)acetic acid was prepared following the procedure described in Step 4 of Example 10, but using ethyl (5-{2-[5-(5-chloro-2-methylphenyl)-3,4,5,6-tetrahydropyrrolo[3,4-c]pyrrol-2(1H)-yl]-1,3-thiazol-5-yl}-2H-tetrazol-2-yl)acetate. The title compound was obtained as a white powder.

¹H NMR (400 MHz, acetone-d₆): δ 7.88 (1H, s), 7.10 (1H, d, J=8.09 Hz), 6.95 (1H, d, J=15.11 Hz), 6.78 (1H, d, J=8.14 Hz), 5.66 (2H, s), 4.42 (4H, s), 4.35 (4H, s), 2.41 (3H, s). MS (ESI, Q⁺) m/z 444 (M+1).

The following additional Examples shown in the Table below were prepared following the procedures outlined in Methods A-G and detailed in Examples 1-11.

| | | MS Data |
|---|---|---|
| Example | Structure | (ESI, Q⁺) |
| 12 | 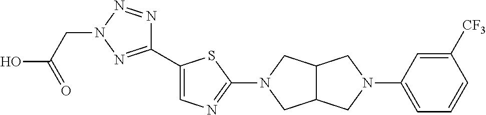 | 466 (M + 1) |
| 13 | 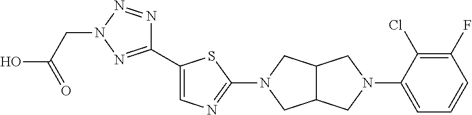 | 450 (M + 1) |
| 14 | 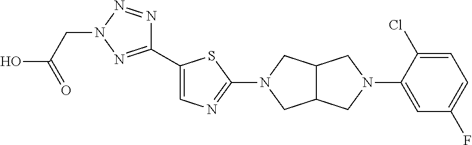 | 450 (M + 1) |
| 15 | 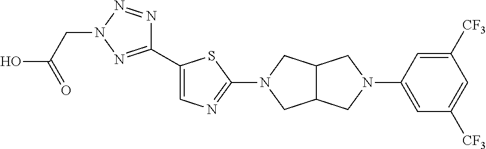 | 534 (M + 1) |
| 16 | 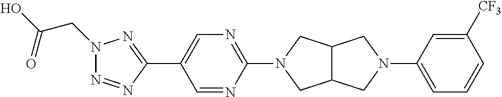 | 461 (M + 1) |
| 17 | 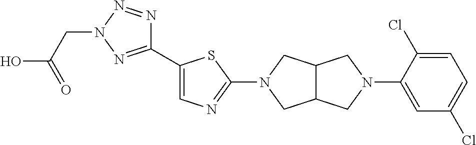 | 466, 468 (M + 1) |
| 18 | 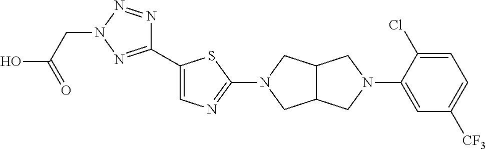 | 500 (M + 1) |
| 19 | 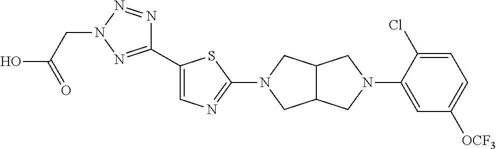 | 516, 518 (M + 1) |
| 20 | 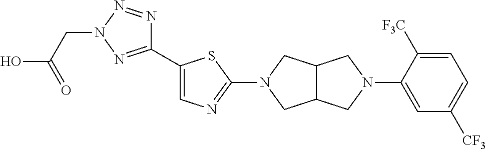 | 534 (M + 1) |
| 21 | 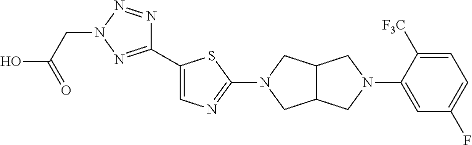 | 484 (M + 1) |
| 22 | 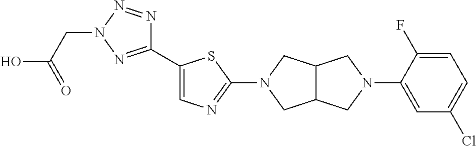 | 450 (M + 1) |
| 23 | 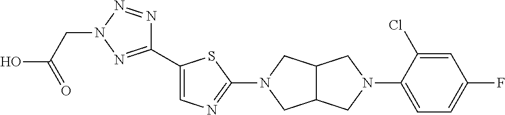 | 450 (M + 1) |
| 24 | 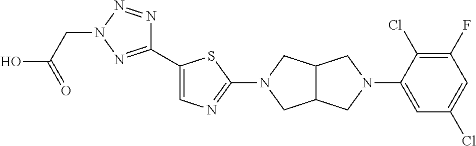 | 484, 486 (M + 1) |
| 25 | 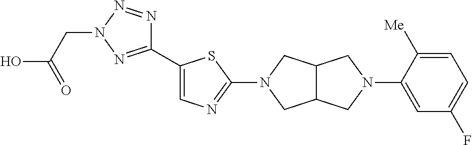 | 430 (M + 1) |
| 26 | 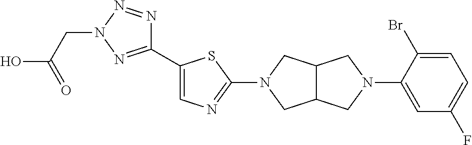 | 494, 496 (M + 1) |
| 27 | 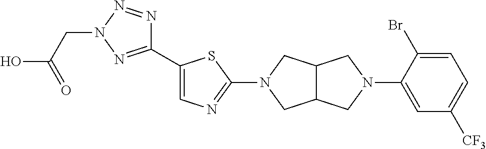 | 544, 546 (M + 1) |
| 28 | 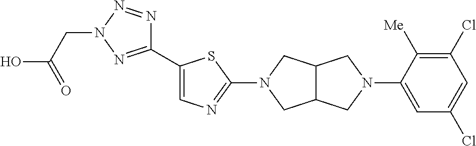 | 480, 482 (M + 1) |
| 29 | 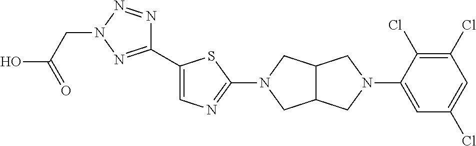 | 500, 502 (M + 1) |
| 30 | 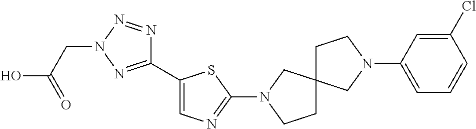 | 446 (M + 1) |
| 31 | 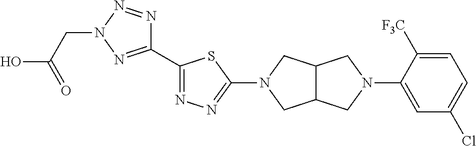 | 501 (M + 1) |
| 32 | 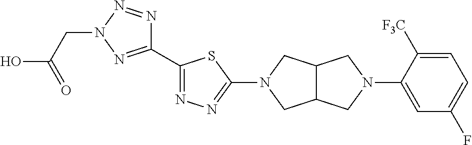 | 485 (M + 1) |
| 33 | 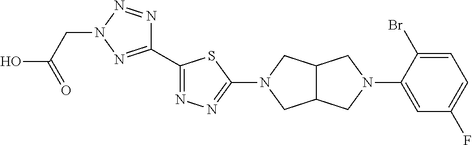 | 495, 497 (M + 1) |
| 34 | 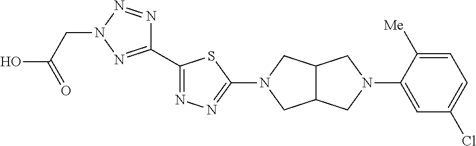 | 447 (M + 1) |
| 35 | 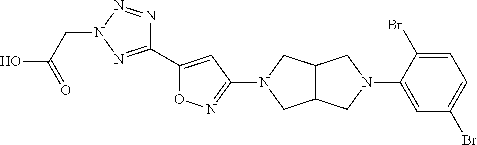 | 540 (M + 1) |
| 36 | 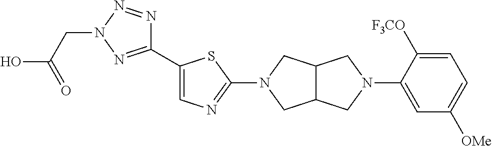 | 512 (M + 1) |
| 37 | 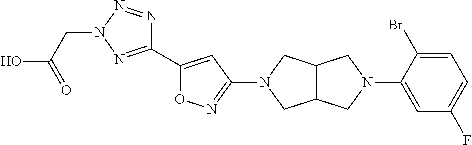 | 478, 480 (M + 1) |
| 38 | 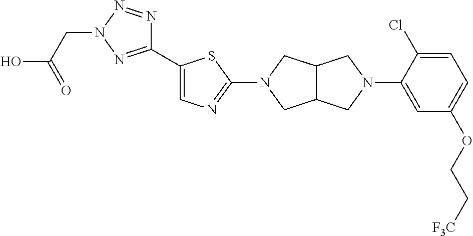 | 544 (M + 1) |
| 39 | 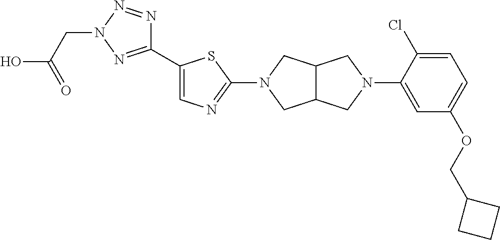 | 516 (M + 1) |
| 40 | 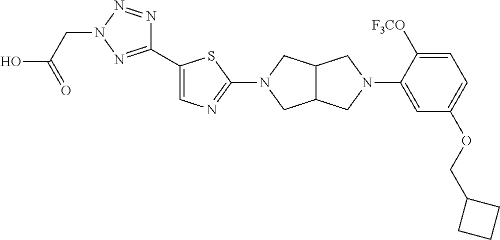 | 566 (M + 1) |
| 41 | 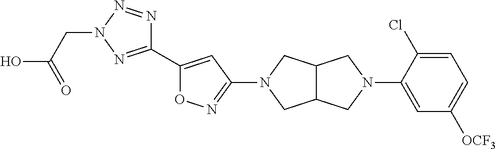 | 500 (M + 1) |
| 42 | 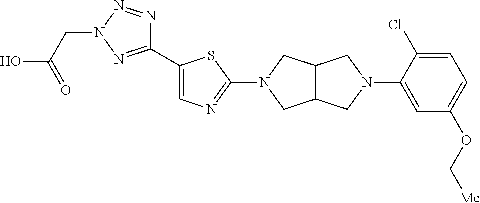 | 476 (M + 1) |
| 43 | 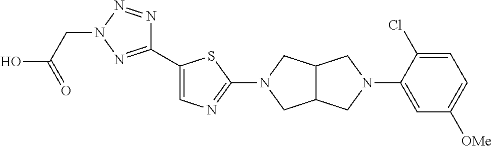 | 462 (M + 1) |
| 44 | 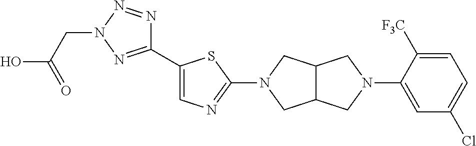 | 500 (M + 1) |
| 45 | 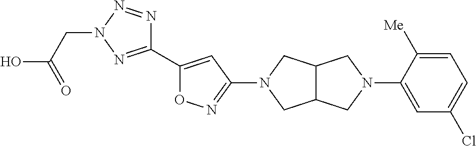 | 430 (M + 1) |
| 46 | 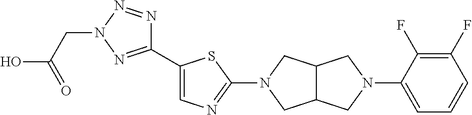 | 434 (M + 1) |
| 47 | 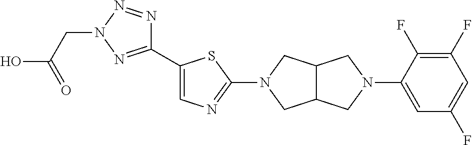 | 452 (M + 1) |
| 48 | 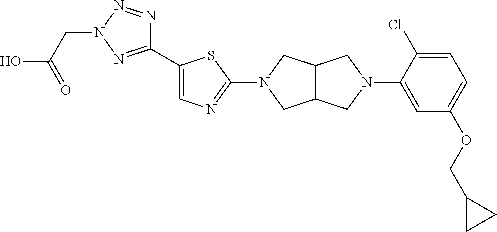 | 502 (M + 1) |
| 49 | 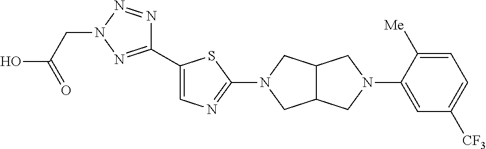 | 480 (M + 1) |
| 50 | 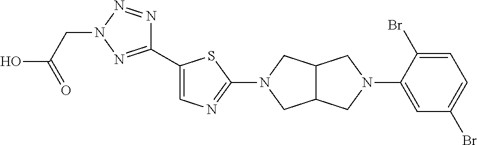 | 556 (M + 1) |
| 51 | 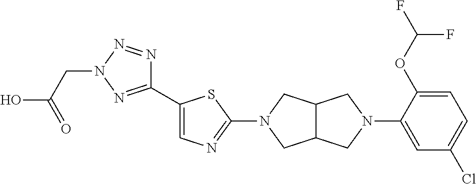 | 498 (M + 1) |
| 52 | 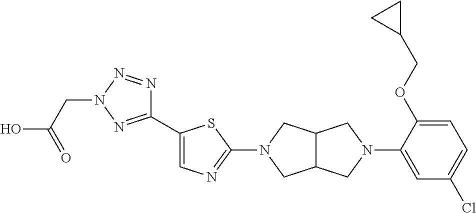 | 502 (M + 1) |
| 53 | 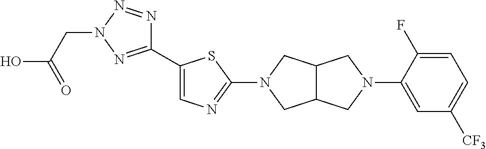 | 484 (M + 1) |
| 54 | 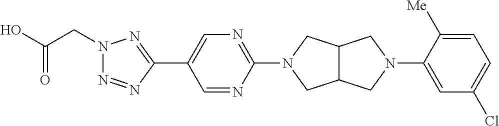 | 441 (M + 1) |
| 55 | 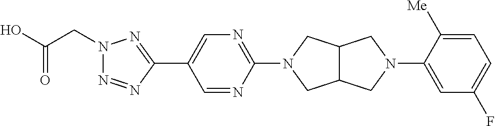 | 425 (M + 1) |
| 56 | 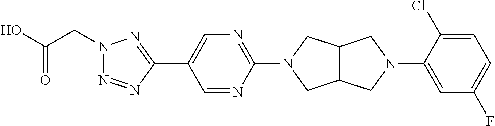 | 445 (M + 1) |
| 57 | 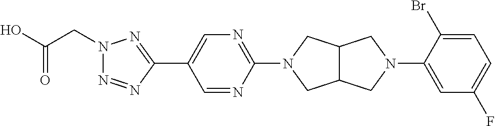 | 489 (M + 1) |
| 58 | 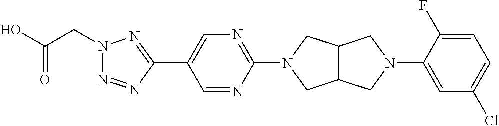 | 445 (M + 1) |
| 59 | 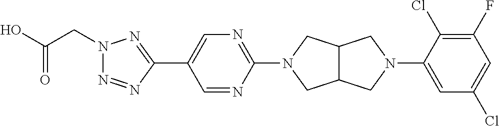 | 479, 481 (M + 1) |
| 60 | 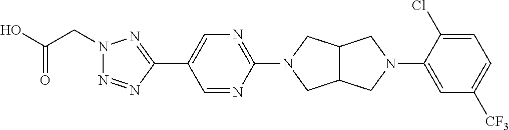 | 495 (M + 1) |
| 61 | 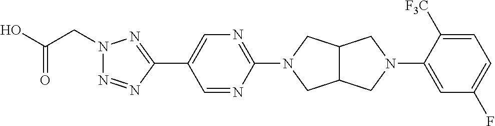 | 479 (M + 1) |
| 62 | 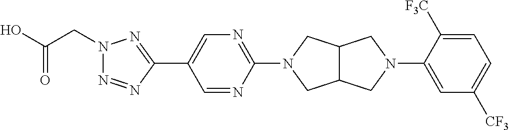 | 529 (M + 1) |
| 63 | 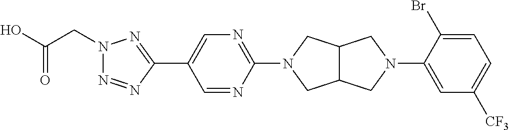 | 539, 541 (M + 1) |
| 64 | 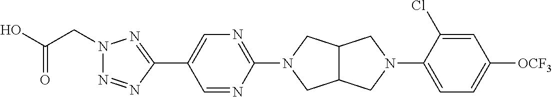 | 511 (M + 1) |
| 65 | 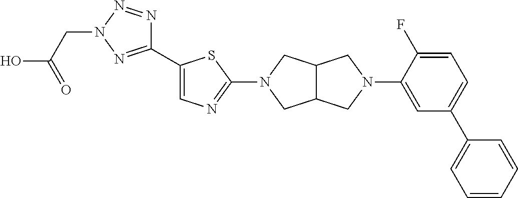 | 492 (M + 1) |
| 66 | 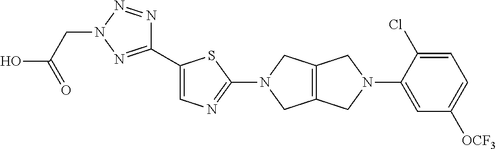 | 514 (M + 1) |
| 67 | 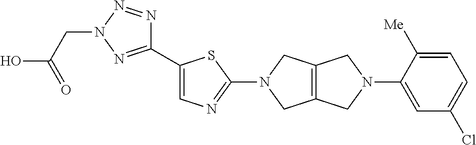 | 444 (M + 1) |

### Examples of Pharmaceutical Formulations

As a specific embodiment of an oral composition of a compound of the present invention, 50 mg of the compound of any of the Examples is formulated with sufficient finely divided lactose to provide a total amount of 580 to 590 mg to fill a size 0 hard gelatin capsule.

As a second specific embodiment of an oral pharmaceutical composition, a 100 mg potency tablet is composed of 100 mg of any one of the Examples, 268 mg microcrystalline cellulose, 20 mg of croscarmellose sodium, and 4 mg of magnesium stearate. The active, microcrystalline cellulose, and croscarmellose are blended first. The mixture is then lubricated by magnesium stearate and pressed into tablets.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

What is claimed is:

1. A compound of structural formula I:
W-Het-Ar (I)
or a pharmaceutically acceptable salts thereof; wherein
Het is a heterobicyclic ring system selected from the group consisting of: 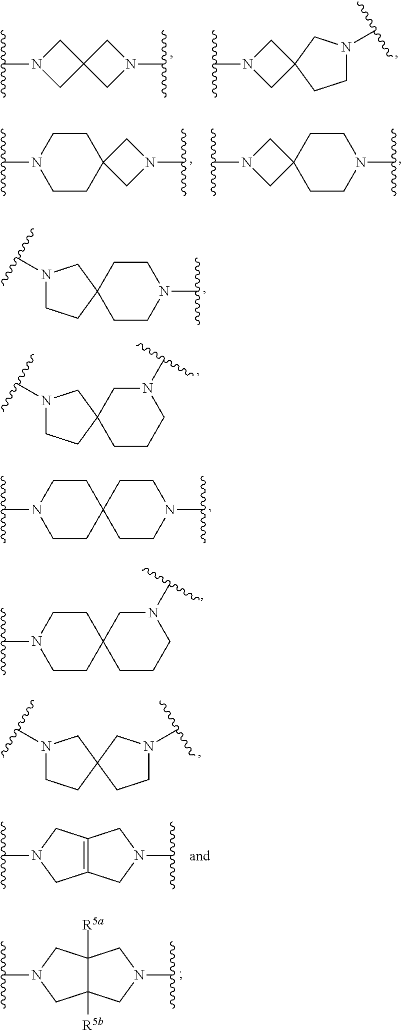
W is heteroaryl selected from the group consisting of: 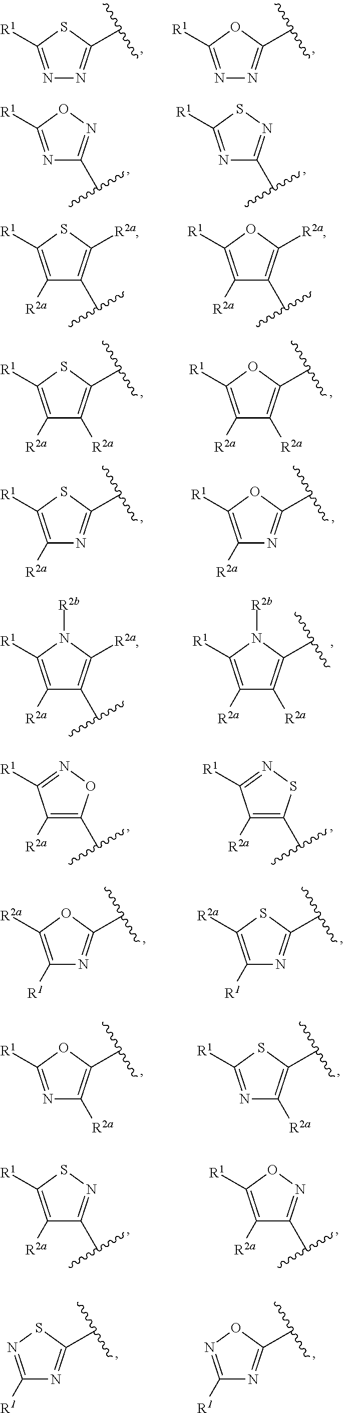 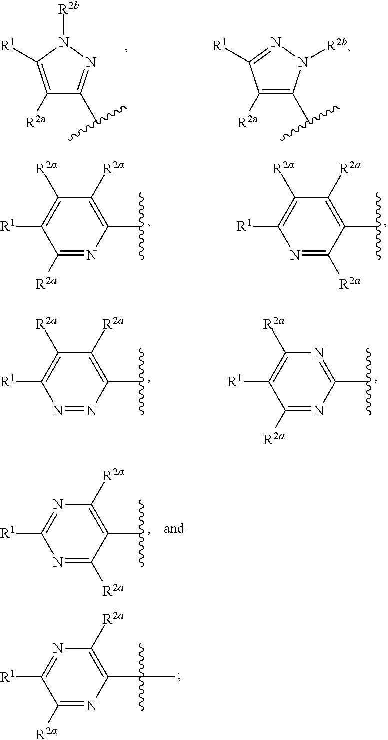
R¹ is heteroaryl selected from the group consisting of: 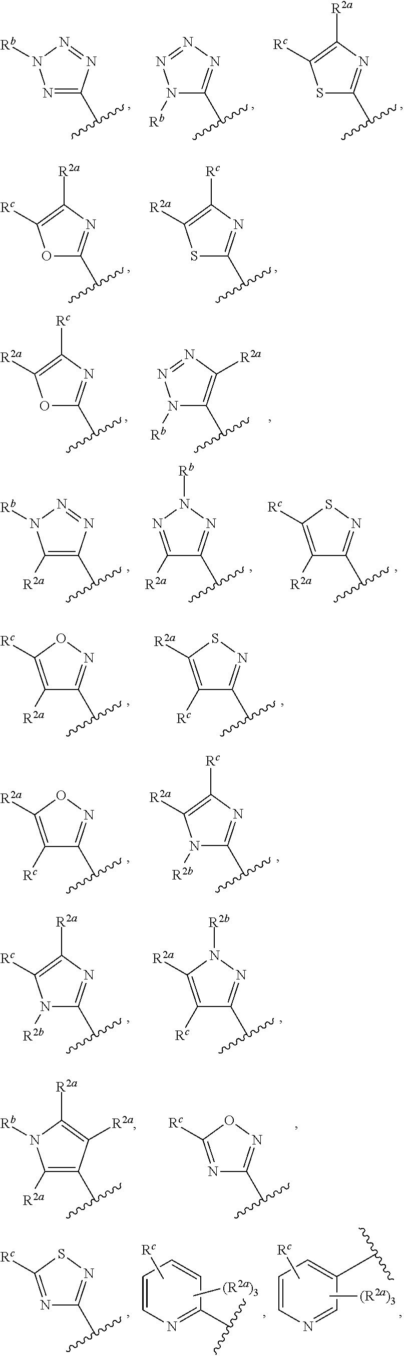 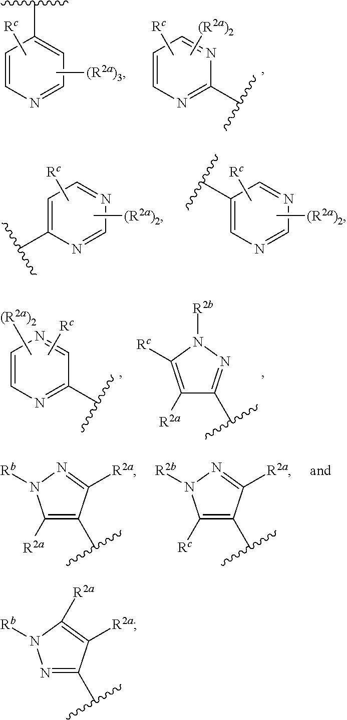
wherein
R^{b} is —(CH₂)ᵣCO₂H, —(CH₂)ᵣCO₂C₁₋₃ alkyl, —(CH₂)ᵣ—Z—(CH₂)ₚCO₂H, or —(CH₂)ᵣ—Z—(CH₂)ₚCO₂C₁₋₃ alkyl;
R^{c} is —(CH₂)ₘCO₂H, —(CH₂)ₘCO₂C₁₋₃ alkyl, —(CH₂)ₘ—Z—(CH₂)ₚCO₂H, or —(CH₂)ₘ—Z—(CH₂)ₚCO₂C₁₋₃ alkyl;
Z is O, S(O)_{q}, or NR⁴;
each R^{2a} is independently selected from the group consisting of:
hydrogen,
halogen,
hydroxy,
cyano,
C₁₋₄ alkyl, optionally substituted with one to five fluorines,
C₁₋₄ alkoxy, optionally substituted with one to five fluorines,
C₁₋₄ alkylthio, optionally substituted with one to five fluorines,
C₁₋₄ alkylsulfonyl, optionally substituted with one to five fluorines, carboxy,
C₁₋₄ alkyloxycarbonyl, and
C₁₋₄ alkylcarbonyl;
each R^{2b} is independently selected from the group consisting of:
hydrogen,
C₁₋₄ alkyl, optionally substituted with one to five fluorines,
C₁₋₄ alkylsulfonyl, optionally substituted with one to five fluorines,
C₁₋₄ alkyloxycarbonyl, and
C₁₋₄ alkylcarbonyl;
Ar is phenyl, naphthyl, thienyl, or pyridyl optionally substituted with one to five R³ substituents;
each R³ is independently selected from the group consisting of:
halogen,
cyano,
C₁₋₆ alkyl, optionally substituted with one to five fluorines,
C₁₋₆ alkoxy, optionally substituted with one to five fluorines,
—OCH₂C₃₋₆ cycloalkyl,
C₁₋₆ alkylthio, optionally substituted with one to five fluorines,
C₁₋₆ alkylsulfonyl, optionally substituted with one to five fluorines, and
phenyl, optionally substituted with one to three substituents independently selected from halogen, C₁₋₄ alkyl, cyano, trifluoromethyl, and trifluoromethoxy;
each R⁴ is independently selected from the group consisting of
hydrogen,
C₁₋₆ alkyl,
(CH₂)ₙ-phenyl,
(CH₂)ₙ-heteroaryl,
(CH₂)ₙ-naphthyl, and
(CH₂)ₙC₃₋₇ cycloalkyl;
wherein alkyl, phenyl, heteroaryl, naphthyl, and cycloalkyl are optionally substituted with one to three groups independently selected from halogen, C₁₋₄ alkyl, and C₁₋₄ alkoxy;
R^{5a} and R^{5b} are each independently selected from the group consisting of:
hydrogen,
fluorine,
hydroxy,
C₁₋₃ alkyl, optionally substituted with one to five fluorines, and
C₁₋₄ alkylcarbonyloxy;
m is an integer from 0 to 3;
n is an integer from 0 to 2;
p is an integer from 1 to 3;
q is an integer from 0 to 2; and
r is an integer from 1 to 3.

2. The compound of claim 1 wherein Het is 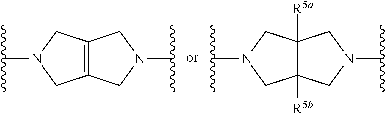
R^{5a} and R^{5b} are as defined in claim 1.

3-5. (canceled)

6. The compound of claim 1 wherein Het is

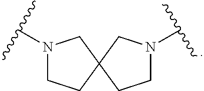

7. The compound of claim 1 wherein Het is selected from the group consisting of:

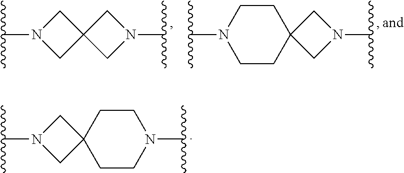

8. The compound of claim 7 wherein Het is

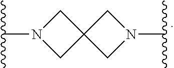

9. The compound of claim 1 wherein Ar is phenyl optionally substituted with one to three substituents each independently selected from R³ as defined in claim 1.

10-12. (canceled)

13. The compound of claim 1 wherein W is heteroaryl selected from the group consisting of:

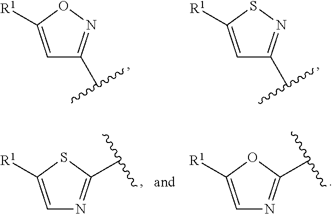

14. The compound of claim 1 wherein W is heteroaryl selected from the group consisting of: 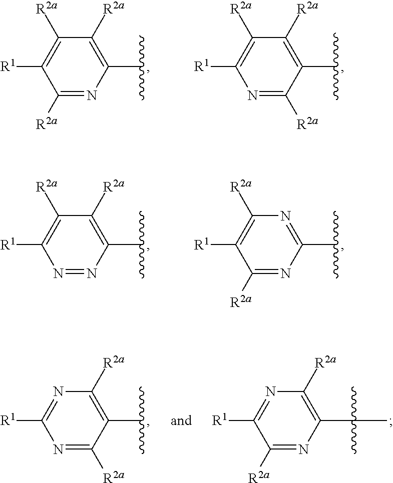
and R¹ and R^{2a} are as defined in claim 1.

15. The compound of claim 14 wherein each R^{2a} is hydrogen.

16. The compound of claim 14 wherein W is

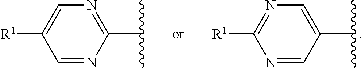

17. The compound of claim 1 wherein R¹ is heteroaryl selected from the group consisting of: 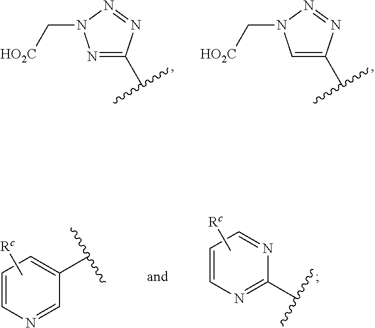
wherein R^{c} is —CO₂H, —CO₂C₁₋₃ alkyl, —CH₂CO₂H, or —CH₂CO₂C₁₋₃ alkyl.

18. The compound of claim 17 wherein R¹ is

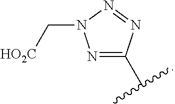

19. The compound of claim 1 wherein W is 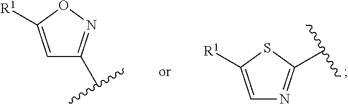
and R¹ is

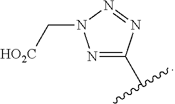

20. The compound of claim 1 wherein Het is 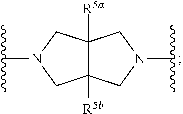
Ar is phenyl optionally substituted with one to three substituents each independently selected from methyl, halogen, trifluoromethyl, and trifluoromethoxy;
W is 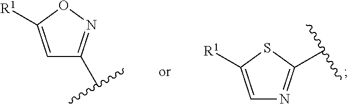
and R¹ is 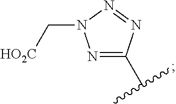
and R^{5a} and R^{5b} are each hydrogen.

21. The compound of claim 1 wherein Het is 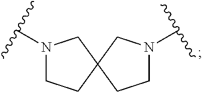
Ar is phenyl optionally substituted with one to three substituents each independently selected from methyl, halogen, trifluoromethyl, and trifluoromethoxy;
W is 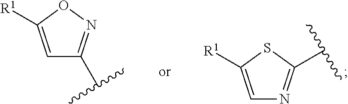
and R¹ is

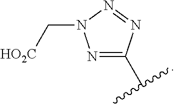

22. The compound of claim 1 wherein Het is selected from the group consisting of: 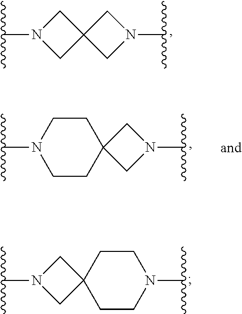
Ar is phenyl optionally substituted with one to three substituents each independently selected from methyl, halogen, trifluoromethyl, and trifluoromethoxy;
W is 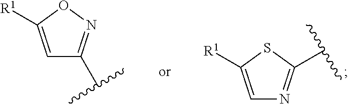
and R¹ is

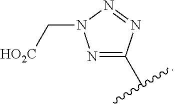

23. (canceled)

24. A compound selected from the group consisting of: 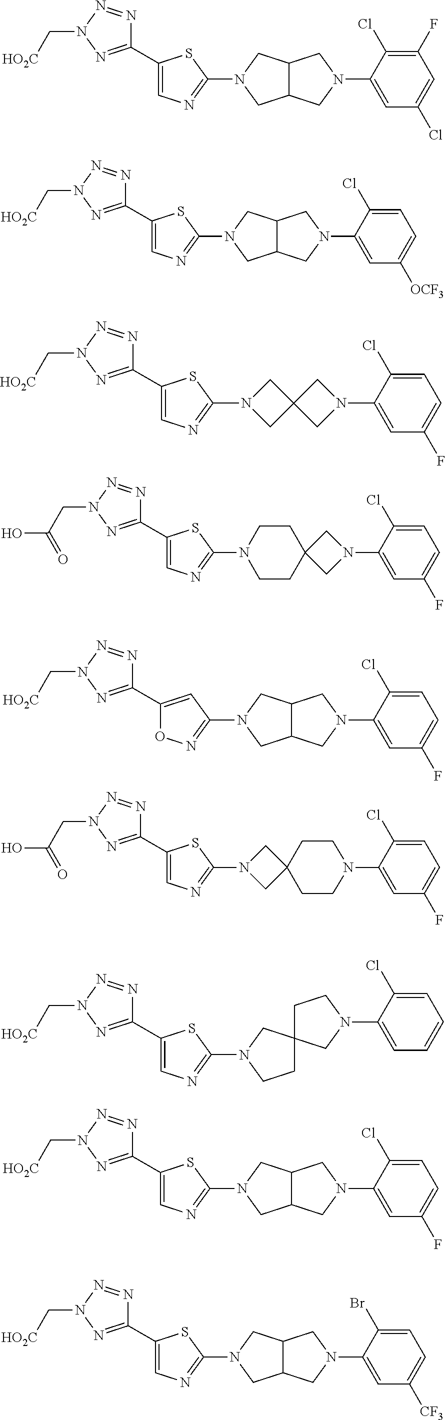 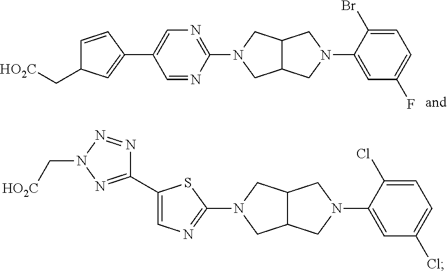
or a pharmaceutically acceptable salt thereof.

25. A pharmaceutical composition comprising a compound in accordance with claim 1 in combination with a pharmaceutically acceptable carrier.

26-30. (canceled)

31. A method of treating hyperglycemia, diabetes or insulin resistance in a mammal in need thereof which comprises the administration to the mammal of a therapeutically effective amount of a compound of claim 1.

32. A method of treating a lipid disorder selected from the group consisting of dyslipidemia, hyperlipidemia, hypertriglyceridemia, hypercholesterolemia, low HDL, and high LDL in a mammal in need thereof which comprises the administration to the mammal of a therapeutically effective amount of a compound of claim 1.
